SEQUENCE LISTING (35 sequences)

SEQ ID NO: 1 - protein, 38 aa, Rattus norvegicus
SGRPPMIVWFNRPFLIAVSHTHGQTILFMAKVINPVGA

SEQ ID NO: 2 - DNA, 17 bp, Artificial Sequence
  Description of Artificial Sequence Synthetic primer
ttcaacmrrccyttyst

SEQ ID NO: 3 - DNA, 18 bp, Artificial Sequence
  Description of Artificial Sequence Synthetic primer
yvacyttkcymakraaga

SEQ ID NO: 4 - protein, 6 aa, Artificial Sequence
  Description of Artificial Sequence Synthetic peptide
FNRPFL

SEQ ID NO: 5 - protein, 8 aa, Artificial Sequence
  Description of Artificial Sequence Synthetic peptide
FMXKVXNP

SEQ ID NO: 6 - protein, 32 aa, Homo sapiens
IVRFNRPFLMIIVPTDTQNIFFMSKVTNPKQA

SEQ ID NO: 7 - protein, 31 aa, Homo sapiens
IVFNRPFLMIIVPTDTQNIFFMSKVTNPKQA

SEQ ID NO: 8 - protein, 31 aa, Homo sapiens; identical to SEQ ID NO: 7

SEQ ID NO: 9 - protein, 32 aa, Homo sapiens
PPVFNKPFVFLMIEQNTKSPLFMGKVVNPTQK

SEQ ID NO: 10 - protein, 30 aa, Homo sapiens
IFNRPFLVVIFSTSTQSVLFLGKVVDPTKP

SEQ ID NO: 11 - protein, 30 aa, Homo sapiens; identical to SEQ ID NO: 10

SEQ ID NO: 12 - protein, 30 aa, Homo sapiens
PFNKPFVFLMIEQNTKSPLFMGKVVNPTQK

SEQ ID NO: 13 - protein, 29 aa, Homo sapiens
FNRPFLVIIKDDITNFPLFIGKVVNPTQK

SEQ ID NO: 14 - protein, 29 aa, Homo sapiens
FNRPFLLLLWEVTTQSLLFLGKVVNPVAG

SEQ ID NO: 15 - protein, 29 aa, Homo sapiens
ANRPFLVFIREVPLNTIIFMGRVANPCVK

SEQ ID NO: 16 - DNA, 133 bp, Homo sapiens
  CDS (23)..(118)
tgcattagtggagacaaggaccattgtgcgtttcaacaggcccttcctgatgatcattgtccctacagacacccagaacatcttcttcatgagcaaagtcaccaatcccaagcaagcctagagcttgccatca

SEQ ID NO: 17 - DNA, 133 bp, Homo sapiens; identical to SEQ ID NO: 16

SEQ ID NO: 18 - DNA, 133 bp, Homo sapiens; identical to SEQ ID NO: 16

SEQ ID NO: 19 - DNA, 98 bp, Homo sapiens
ccccagaggtcaagttcaacaaaccctttgtcttcttaatgattgaacaaaataccaagtctcccctcttcatgggaaaagtggtgaatcccacccaa

SEQ ID NO: 20 - DNA, 118 bp, Homo sapiens
tgcccagaccaatcgccacatcctgcgattcaaccggcccttccttgtggtgatcttttccaccagcacccagagtgtcctctttctgggcaaggtcgtcgaccccacgaaaccatag

SEQ ID NO: 21 - DNA, 145 bp, Homo sapiens
tgcccagaccaatcgccacatcctgcgattcaaccggcccttccttgtggtgatcttttccaccagcacccagagtgtcctctttctgggcaaggtcgtcgaccccacgaaaccatagccctcccagggctgctcatctgttcca

SEQ ID NO: 22 - DNA, 103 bp, Homo sapiens
tatcccccccgaggtcaagttcaacaaaccctttgtcttcttaatgattgaacaaaataccaagtctcccctcttcatgggaaaagtggtgaatcccacccaa

SEQ ID NO: 23 - DNA, 101 bp, Homo sapiens
agtatcagacagtcatgttcaaccggcccttcctggtcatcatcaaggatgacatcaccaactttccgctcttcattggaaaagtggtgaatcccacccaa

SEQ ID NO: 24 - DNA, 113 bp, Homo sapiens
cccatctctgaacaccatgtcagacccacatgcccacttcaacaggcctttcctcttgctcctttgggaggtcaccacccagagcttactcttcctgggaaaagttgtcaacc

SEQ ID NO: 25 - protein, 12 aa, Rattus norvegicus
KKDDTDDEIAKY

SEQ ID NO: 26 - protein, 17 aa, Rattus norvegicus
KNKGDEEEEEEKLEEKQ

SEQ ID NO: 27 - protein, 12 aa, Rattus norvegicus
RKDSETGENIRQ

SEQ ID NO: 28 - protein, 9 aa, Rattus norvegicus
KLKEEISKM

SEQ ID NO: 29 - protein, 11 aa, Rattus norvegicus
KVLENAEGART

SEQ ID NO: 30 - protein, 10 aa, Rattus norvegicus
KNQIGDKEKL

SEQ ID NO: 31 - protein, 9 aa, Rattus norvegicus
KFAEEDKKL

SEQ ID NO: 32 - protein, 11 aa, Rattus norvegicus
KVLEDSDLKKS

SEQ ID NO: 33 - protein, 591 aa, Rattus norvegicus
MEGKWLLCLLLVLGTAAIQAHDGHDDDMIDIEDDLDDVIEEVEDSKSKSDTSTPPSPKVTYKAPVPTGEVYFADSFDRGSLSGWILSKAKKDDTDDEIAKYDGKWEVDEMKETKLPGDKGLVLMSRAKHHAISAKLNKPFLFDTKPLIVQYEVNFQNGIECGGAYVKLLSKTSELNLDQFHDKTPYTIMFGPDKCGEDYKLHFIFRHKNPKTGVYEEKHAKRPDADLKTYFTDKKTHLYTLILNPDNSFEILVDQSVVNSGNLLNDMTPPVNPSREIEDPEDRKPEDWDERPKIADPDAVKPDDWDEDAPSKIPDEEATKPEGWLDDEPEYIPDPDAEKPEDWDEDMDGEWEAPQIANPKCESAPGCGVWQRPMIDNPNYKGKWKPPMIDNPNYQGIWKPRKIPNPDFFEDLEPFRMTPFSAIGLELWSMTSDIFFDNFIISGDRRVVDDWANDGWGLKKAADGAAEPGVVGQMLEAAEERPWLWVVYILTVALPVFLVILFCCSGKKQSNAMEYKKTDAPQPDVKDEEGKEEEKNKGDEEEEEEKLEEKQKSDAEEDGGTGSQDEEDSKPKAEEDEILNRSPRNRKPRRE

SEQ ID NO: 34 - protein, 10 aa, Rattus norvegicus
KDDTDDEIAK

SEQ ID NO: 35 - protein, 15 aa, Rattus norvegicus
NKGDEEEEEEKLEEK

(19) United States
(12) Patent Application Publication
(10) Pub. No.: US 2008/0300170 A1
(43) Pub. Date: Dec. 4, 2008

(54) Compositions and methods for diagnosis and treatment for type 2 diabetes

(76) Inventors: Cohava Gelber, Nokesville, VA (US); Liping Liu, Manassas, VA (US); Zhidong Xie, Manassas, VA (US); Pranvera Ikonomi, Manassas, VA (US); John R. Simms, Haymarket, VA (US); Catherine R. Auge, Haymarket, VA (US)

Correspondence Address: MINTZ LEVIN COHN FERRIS GLOVSKY & POPEO;ATTN: PATENT INTAKE CUSTOMER NO. 35437, ONE FINANCIAL CENTER, BOSTON, MA, 02111, US

(21) Appl. No.: 11/901,925

(22) Filed: Sep. 18, 2007

Related U.S. Application Data
(63) Continuation-in-part of application No. PCT/US2007/007875, filed on Mar. 28, 2007.
(60) Provisional application No. 60/841,717, filed on Sep. 1, 2006.

(51) Int. Cl. A61K 38/28; G01N 33/53; C07K 16/18; A61P 3/10; C12Q 1/68

(52) U.S. Cl. 514/3; 435/7.1; 435/6; 530/387.9

(57) ABSTRACT

The present invention relates generally to the identification of biological markers associated with an increased risk of developing Diabetes, as well as methods of using such biological markers in diagnosis and prognosis of Diabetes. The biological markers of the invention may indicate new targets for therapy or constitute new therapeutics for the treatment or prevention of Diabetes.

### CROSS-REFERENCE TO RELATED APPLICATIONS

This application is a continuation-in-part of International Application No. PCT/US2007/007875, filed on Mar. 28, 2007, which claims priority from U.S. Provisional Application Ser. No. 60/841,717, filed on Sep. 1, 2006.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates generally to the identification of biological markers associated with an increased risk of developing Diabetes, as well as methods of using such biological markers in diagnosis and prognosis of Diabetes. Furthermore, selected biological markers of the present invention present new targets for therapy and constitute new therapeutics for treatment or prevention of Diabetes.

### BACKGROUND OF THE INVENTION

Diabetes mellitus comprises a cluster of diseases distinguished by chronic hyperglycemia that result from the body's failure to produce and/or use insulin, a hormone produced by β-cells in the pancreas that plays a vital role in metabolism. Symptoms include increased thirst and urination, hunger, weight loss, chronic infections, slow wound healing, fatigue, and blurred vision. Often, however, symptoms are not severe, not recognized, or are absent. Diabetes can lead to debilitating and life-threatening complications including retinopathy leading to blindness, memory loss, nephropathy that may lead to renal failure, cardiovascular disease, neuropathy, autonomic dysfunction, and limb amputation. Several pathogenic processes are involved in the development of Diabetes, including but not limited to, processes which destroy the insulin-secreting β-cells with consequent insulin deficiency, and changes in liver and smooth muscle cells that result in resistance to insulin uptake. Diabetes can also comprise abnormalities of carbohydrate, fat, and protein metabolism attributed to the deficient action of insulin on target tissues resulting from insulin insensitivity or lack of insulin.

Type 2 Diabetes is the most common form of Diabetes, which typically develops as a result of a relative, rather than absolute, insulin deficiency, in combination with the body's failure to use insulin properly (also known in the art as “insulin resistance”). Type 2 Diabetes often manifests in persons, including children, who are overweight; other risk factors include high cholesterol, high blood pressure, ethnicity, and genetic factors, such as a family history of Diabetes. The majority of patients with Type 2 Diabetes are obese, and obesity itself may cause or aggravate insulin resistance. Apart from adults, an increasing number of children are also being diagnosed with Type 2 Diabetes. Due to the progressive nature of the disease, Diabetes complications often develop by the time these children become adults. A study by the American Diabetes Association (ADA) involved 51 children that were diagnosed with Diabetes before the age of 17. By the time these children reached their early 30s, three had kidney failure, one was blind, and two died of heart attacks while on dialysis. This study reinforces the severity of the disease, the serious damage inflicted by Diabetes complications, and the need for early diagnosis of the disease.

The incidence of Diabetes has been rapidly escalating to alarming numbers. Diabetes currently affects approximately 170 million people worldwide with the World Health Organization (WHO) predicting 300 million diabetics by 2025. The United States alone has 20.8 million people suffering from Diabetes (approximately 6% of population and the 6^{th} most common cause of death). The annual direct healthcare costs of Diabetes worldwide for people in the 20-79 age bracket are estimated at $153-286 billion and is expected to rise to $213-396 billion in 2025.

Along with the expansion of the diagnosed diabetic population, the undiagnosed diabetic population has also continued to increase, primarily because Type 2 Diabetes is often asymptomatic in its early stages, or the hyperglycemia is often not severe enough to provoke noticeable symptoms of Diabetes. It is believed that approximately 33% of the 20.8 million diabetics in the United States remain undiagnosed. Due to the delay in diagnosis, Diabetes complications have already advanced and thus, the future risk of further complication and derailment is severely increased. To obviate complications and irreversible damage to multiple organs, Diabetes management guidelines advocate initiation of therapeutic intervention early in the prognosis of the disease.

This modem epidemic requires new tools for early detection of Type 2 Diabetes, before the disease instigates significant and irreparable damage. In addition, new treatment paradigms are needed to halt, delay, or ameliorate the massive deterioration in patient health, ideally reversing the course of the disease to partial or complete cure as an alternative or a substitute for current treatments, which merely address chronic management of disease symptoms. Diabetic hyperglycemia can be decreased by weight reduction, increased physical activity, and/or therapeutic treatment modalities. Several biological mechanisms are associated with hyperglycemia, such as insulin resistance, insulin secretion, and gluconeogenesis, and there are several agents available that act on one or more of these mechanisms, such as but not limited to metformin, acarbose, and rosiglitazone.

It is well documented that the pre-diabetic state can be present for ten or more years before the detection of glycemic disorders like Diabetes. Treatment of pre-diabetics with therapeutic agents can postpone or prevent Diabetes; yet few pre-diabetics are identified and treated. A major reason, as indicated above, is that no simple laboratory test exists to determine the actual risk of an individual to develop Diabetes. Thus, there remains a need in the art for methods of identifying and diagnosing these individuals who are not yet diabetics, but who are at significant risk of developing Diabetes.

### SUMMARY OF THE INVENTION

The present invention is premised on the discovery that disease-associated biomarkers can be identified in serum or other bodily fluids long before overt disease is apparent. The presence or absence of these biomarkers from the serum footprints of patients suffering from Type 2 Diabetes precede disruptions in blood glucose control and can be used as early diagnostic tools, for which treatment strategies can be devised and administered to prevent, delay, ameliorate, or reverse irreversible organ damage. One or several of the disease-associated biomarkers of the present invention can be used to diagnose subjects suffering from Type 2 Diabetes or related diseases, or advantageously, to diagnose those subjects who are asymptomatic for Type 2 Diabetes and related diseases. The biomarkers of the present invention can also be used for the design of new therapeutics. For instance, a biomarker absent in a diabetic patient and found in a healthy individual can constitute a new protective or therapeutic agent which, upon administration to the patient, may alleviate symptoms or even reverse the disease.

Accordingly, in one aspect, the present invention provides a method of diagnosing or identifying type 2 Diabetes, one or more complications related to type 2 Diabetes, or a pre-diabetic condition in a subject, comprising measuring an effective amount of one or more T2DBMARKERS or a metabolite thereof in a sample from the subject, and comparing the amount to a reference value, wherein an increase or decrease in the amount of the one or more T2DBMARKERS relative to the reference value indicates that the subject suffers from the type 2 Diabetes, one or more complications related to type 2 Diabetes, or the pre-diabetic condition.

In one embodiment, the reference value comprises an index value, a value derived from one or more Diabetes risk prediction algorithms or computed indices, a value derived from a subject not suffering from type 2 Diabetes or a pre-diabetic condition, or a value derived from a subject diagnosed with or identified as suffering from type 2 Diabetes or a pre-diabetic condition, or a value derived from a subject previously diagnosed with or identified as suffering from one or more complications related to type 2 Diabetes.

In another embodiment, the decrease is at least 10% greater than the reference value. In other embodiments, the increase is at least 10% greater than the reference value.

The sample can be urine, serum, blood plasma, blood cells, endothelial cells, tissue biopsies, pancreatic juice, ascites fluid, bone marrow, interstitial fluid, tears, sputum, or saliva.

The T2DBMARKERS of the present invention can be detected electrophoretically, immunochemically, by proteomics technology, or by genomic analysis. The immunochemical detection can be radioimmunoassay, immunoprecipitation, immunoblotting, immunofluorescence assay, or enzyme-linked immunosorbent assay. The proteomics technology can comprise SELDI, MALDI, LC/MS, tandem LC/MS/MS, protein/peptide arrays, or antibody arrays. The genomic analysis can comprise polymerase chain reaction (PCR), real-time PCR, microarray analysis, Northern blotting, or Southern blotting. Preferably, the T2DBMARKERS disclosed herein are detected immunochemically using the isolated antibodies of the present invention, mentioned elsewhere in this disclosure.

In another embodiment, the subject has not been previously diagnosed as having type 2 Diabetes, one or more complications related to type 2 Diabetes, or a pre-diabetic condition. The subject can also be one who has been previously diagnosed as having type 2 Diabetes, one or more complications related to type 2 Diabetes, or a pre-diabetic condition. Alternatively, the subject can be asymptomatic for the type 2 Diabetes, one or more complications related to type 2 Diabetes, or the pre-diabetic condition.

Another aspect of the present invention provides a method for monitoring the progression of type 2 Diabetes, one or more complications relating to type 2 Diabetes, or a pre-diabetic condition in a subject, comprising (a) detecting an effective amount of one or more T2DBMARKERS in a first sample from the subject at a first period of time, (b) detecting an effective amount of one or more T2DBMARKERS in a second sample from the subject at a second period of time, and (c) comparing the amounts of the one or more T2DBMARKERS detected in step (a) to the amount detected in step (b), or to a reference value. The monitoring can comprise evaluating changes in the risk of developing type 2 Diabetes, one or more complications related to type 2 Diabetes, or the pre-diabetic condition.

In one embodiment, the subject can comprise one who has previously been treated for the type 2 Diabetes, one or more complications related to type 2 Diabetes, or the pre-diabetic condition. Alternatively, the subject can be one who has not been previously treated for the type 2 Diabetes, one or more complications related to type 2 Diabetes, or the pre-diabetic condition, or one who has not been previously diagnosed with or identified as suffering from type 2 Diabetes, one or more complications related to type 2 Diabetes, or the pre-diabetic condition.

In another embodiment, the first sample is taken from the subject prior to being treated for the type 2 Diabetes, one or more complications related to type 2 Diabetes, or the pre-diabetic condition. The second sample can be taken from the subject after being treated for the type 2 Diabetes, one or more complications related to type 2 Diabetes, or the pre-diabetic condition. In another embodiment, the monitoring can further comprise selecting a treatment regimen for the subject and/or monitoring the effectiveness of a treatment regimen for type 2 Diabetes, one or more complications related to type 2 Diabetes, or the pre-diabetic condition.

In other embodiments, the treatment for the type 2 Diabetes, one or more complications related to type 2 Diabetes, or the pre-diabetic condition comprises exercise regimens, dietary supplements, surgical intervention, diabetes-modulating agents, or combinations thereof. The progression of type 2 Diabetes, Diabetes complications, or pre-diabetic conditions can be monitored by detecting changes in body mass index (BMI), insulin levels, blood glucose levels, HDL levels, systolic and/or diastolic blood pressure, or combinations thereof.

In another aspect of the present invention, a method of monitoring the effectiveness of a treatment regimen for type 2 Diabetes, one or more complications related to type 2 Diabetes, or a pre-diabetic condition in a subject is provided, comprising (a) detecting an effective amount of one or more T2DBMARKERS in a first sample from the subject prior to treatment of the type 2 Diabetes, one or more complications related to type 2 Diabetes, or the pre-diabetic condition, (b) detecting an effective amount of one or more T2DBMARKERS in a second sample from the subject after treatment of the type 2 Diabetes, one or more complications related to type 2 Diabetes, or the pre-diabetic condition, and (c) comparing the amount of the one or more T2DBMARKERS detected in step (a) to the amount detected in step (b), or to a reference value. In one embodiment, changes in blood glucose levels can be detected by oral glucose tolerance test.

Yet another aspect of the present invention provides a method of treating a subject diagnosed with or identified as suffering from type 2 Diabetes, one or more complications related to type 2 Diabetes, or a pre-diabetic condition, comprising detecting an effective amount of one or more T2DBMARKERS or metabolites thereof present in a first sample from the subject at a first period of time, and treating the subject with one or more diabetes-modulating agents until the amounts of the one or more T2DBMARKERS or metabolites thereof return to a reference value measured in one or more subjects at low risk for developing type 2 Diabetes, one or more complications related to type 2 Diabetes, or a pre-diabetic condition, or a reference value measured in one or more subjects who show improvements in Diabetes risk factors as a result of treatment with the one or more diabetes-modulating agents.

In one embodiment, the one or more diabetes-modulating agents comprise sulfonylureas, biguanides, insulin, insulin analogs, peroxisome prolifereator-activated receptor-γ (PPAR-γ) agonists, dual-acting PPAR agonists, insulin secretagogues, analogs of glucagon-like peptide-1 (GLP-1), inhibitors of dipeptidyl peptidase IV, pancreatic lipase inhibitors, α-glucosidase inhibitors, or combinations thereof. In another embodiment, the improvements in Diabetes risk factors as a result of treatment with one or more diabetes-modulating agents comprise a reduction in body mass index (BMI), a reduction in blood glucose levels, an increase in insulin levels, an increase in HDL levels, a reduction in systolic and/or diastolic blood pressure, or combinations thereof.

In another aspect of the present invention, a method of selecting a treatment regimen for a subject diagnosed with or identified as suffering from type 2 Diabetes, one or more complications related to type 2 Diabetes, or a pre-diabetic condition is provided, comprising (a) detecting an effective amount of one or more T2DBMARKERS in a first sample from the subject at a first period of time, (b) detecting an effective amount of one or more T2DBMARKERS in a second sample from the subject at a second period of time, and comparing the amounts of the one or more T2DBMARKERS detected in step (a) to the amount detected in step (b), or to a reference value. In one embodiment, the reference value is derived from one or more subjects who show an improvement in Diabetes risk factors as a result of one or more treatments for type 2 Diabetes, one or more complications related to type 2 Diabetes, or the pre-diabetic condition.

Another aspect of the present invention provides a method of evaluating changes in the risk of developing type 2 Diabetes, one or more complications related to type 2 Diabetes, or a pre-diabetic condition in a subject, comprising (a) detecting an effective amount of one or more T2DBMARKERS in a first sample from the subject at a first period of time, (b) detecting an effective amount of one or more T2DBMARKERS in a second sample from the subject at a second period of time, and comparing the amounts of the one or more T2DBMARKERS detected in step (a) to the amount detected in step (b), or to a reference value.

In another aspect, a method of identifying one or more complications related to type 2 Diabetes in a subject is provided, comprising measuring an effective amount of one or more T2DBMARKERS or a metabolite thereof in a sample from the subject and comparing the amount to a reference value, wherein an increase or decrease in the amount of the one or more T2DBMARKERS relative to the reference value indicates that the subject suffers from or is at risk for developing complications related to type 2 Diabetes.

In one embodiment, the complications comprise retinopathy, blindness, memory loss, nephropathy, renal failure, cardiovascular disease, neuropathy, autonomic dysfunction, hyperglycemic hyperosmolar coma, or combinations thereof. In another embodiment, the reference value comprises an index value, a value derived from one or more diabetes risk-prediction algorithms or computed indices, a value derived from a subject diagnosed with or identified as suffering from type 2 Diabetes or a value derived from a subject previously identified as having one or more complications related to type 2 Diabetes.

Another aspect of the present invention provides a type 2 Diabetes reference expression profile, comprising a pattern of expression levels of one or more T2DBMARKERS detected in one or more subjects who are not diagnosed with or identified as suffering from type 2 Diabetes. In another aspect, the present invention provides a pre-diabetic condition reference expression profile, comprising a pattern of expression levels of one or more T2DBMARKERS detected in one or more subjects who are not diagnosed with or identified as suffering from a pre-diabetic condition. The invention also provides a type 2 Diabetes subject expression profile, comprising a pattern of expression levels detected in one or more subjects diagnosed with or identified as suffering from type 2 Diabetes, are at risk for developing type 2 Diabetes, or are being treated for type 2 Diabetes. In another aspect, the present invention also provides a pre-diabetic condition subject expression profile, comprising a pattern of expression levels detected in one or more subjects diagnosed with or identified as suffering from a pre-diabetic condition, are at risk for developing a pre-diabetic condition, or are being treated for a pre-diabetic condition.

The present invention also provides a kit comprising T2DBMARKER detection reagents that detect one or more T2DBMARKERS, a sample derived from a subject having normal glucose levels, and optionally instructions for using the reagents in any of the methods of the present invention described herein, wherein the T2DBMARKER detection reagents can comprise, for example, the isolated antibody of the invention. The detection reagents can further comprise, for example, one or more antibodies or fragments thereof, one or more aptamers, one or more oligonucleotides, or combinations thereof.

In another aspect of the present invention, a pharmaceutical composition for treating type 2 Diabetes or a pre-diabetic condition in a subject is provided, comprising a therapeutically effective amount of one or more T2DBMARKERS or a metabolite thereof, and a pharmaceutically acceptable carrier or diluent. In some embodiments, the T2DBMARKER metabolite comprises SEQ ID NO: 1. In other embodiments, the T2DBMARKER metabolite comprises at least 5, at least 10, at least 15, or at least 20 contiguous amino acid residues of SEQ ID NO: 1. Alternatively, the T2DBMARKER metabolite can comprise an amino acid sequence at least 90% identical to SEQ ID NO: 1.

The present invention also provides a pharmaceutical composition consisting essentially of SEQ ID NO: 1 and a pharmaceutically acceptable carrier or diluent.

In yet another aspect, a method of treating type 2 Diabetes or a pre-diabetic condition in a subject in need thereof is provided, comprising administering to the subject a therapeutically effective amount of the pharmaceutical compositions of the invention.

The present invention further provides an isolated antibody or antigen-binding fragment thereof, comprising a human constant region and an antigen-binding region, wherein the antigen-binding region binds one or more T2DBMARKERS or a metabolite thereof. Preferably, the isolated antibody of the invention contains an antigen-binding region that binds one or more amino acid residues of SEQ ID NO: 1. In some embodiments, the isolated antibody can be recombinant. The isolated antibodies or antigen-binding fragments of the invention can be used in any of the methods disclosed herein, for detection of one or more T2DBMARKERS set forth in Table 1.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, materials, methods, and examples described herein are illustrative only and are not intended to be limiting.

Other features and advantages of the invention will be apparent from and are encompassed by the following detailed description and claims.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 represents a protein expression profile of pancreatic extracts from Cohen diabetic resistant (CDr) and sensitive (CDs) rats fed regular diet (RD) or copper-poor high-sucrose diet (HSD). Total protein extract (5 μg) was prepared under reducing conditions and run on a 4-12% polyacrylamide gel.

FIG. 2A is a graphical comparison of serum samples from CDr-RD, CDs-RD, CDr-HSD, and CDs-HSD on a SELDI Q10 anion exchange surface chip. A median peak is present in CDr-RD and CDr-HSD (marked by an arrow), but not in CDs-RD and CDs-HSD. A protein fragment from this differentially expressed peak was identified as the C-terminal fragment of Serpina 3M.

FIG. 2B is an MS/MS spectrum of the 4.2 kilodalton fragment identified by SELDI.

FIG. 3A depicts a BLAST alignment of the 38-amino acid Serpina 3M (also referred to as “D3”) peptide and proteins identified as having similar sequence identity.

FIG. 3B shows a BLAST alignment of nucleic acid sequences encoding the 38-amino acid Serpina 3M peptide and proteins identified in 3A.

FIG. 3C is a photograph of an agarose gel displaying the results of an RT-PCR experiment using degenerate primers designed to detect the conserved amino acid motifs found in the BLAST alignments of FIGS. 3A and 3B.

FIG. 4A is a photograph of two-dimensional maps of CDr-RD, CDs-RD, CDr-HSD and CDs-HSD serum samples analyzed by the 2D/LC fractionation system. The intensity of the blue bands represents the relative protein amount as detected at 214 nm by UV absorbance.

FIG. 4B shows a differential second-dimensional reverse-phase HPLC elution profile of CDr-RD (red) versus CDs-RD (green) of a selected first-dimensional isoelectric point fraction (Fraction 31). Proteins that were uniquely identified in CDs-RD samples are listed at the bottom of the graph.

FIG. 5A is a photograph of a protein gel representing differential protein profiling of CD rat serum samples using two-dimensional gel electrophoresis (2DE). The pH for the first dimension chromatofocusing was from pH 5-8, and the second dimensional separation used a 4-20% Tris-HCl SDS-PAGE gel. The gel was stained with BioSafe Coomassie Staining (Bio-Rad) for visualization.

FIG. 5B is a magnified view of the spots identified in FIG. 5A.

FIG. 6 comprises graphical representations illustrating differentially expressed proteins found in the Cohen Diabetic rat models using 2DE.

FIG. 7 is a histogram depicting the differentially expressed Cohen Diabetic rat serum proteins identified by 2DE.

FIG. 8 is a photograph of Western blots depicting the reactivity of the D3-hyperimmune rabbit serum with the ˜4 kD protein fragment present in CDr-RD and CDr-HSD rat serum. In the left photograph, a higher molecular weight doublet (in the range of 49 and 62 kD) also reacted with the hyperimmune sera, indicating that a parent protein (and a protein complex) is expressed by all strains under both RD and HSD treatment modalities, while the derivative of smaller size is differentially expressed only in the CDr strain. As a negative control, the right photograph shows a Western blot membrane incubated in the absence of the D3 hyperimmune rabbit serum.

FIG. 9 depicts the concentration of the D3 peptide in CDr rat serum as calculated from SELDI analysis.

FIG. 10 are photographs of gels containing liver extracts (10 μg), which was probed with secondary goat anti-rabbit IgG conjugated to horseradish peroxidase (HRP)(1:25000 dilution), in the presence (right panel) or absence (left panel) of primary anti-D3 serum antibody (1:200 dilution).

FIG. 11 is a photograph of a Western blot analyzing human sera using D3 hyperimmune serum from rabbits. Lane 1 corresponds to the molecular weight marker. Lanes 2-7 represent fractions of a single serum sample from a normal individual (3045 NGT). Lanes 10-14 represent fractions of a single serum sample from a Type 2 Diabetes patient (291).

FIGS. 12A and 12B show preparative gels that were run with 100 μg of CDr-HSD and CDs-HSD pancreatic extracts, respectively. The positive control was stained with 20 μg of anti-actin antibody, and the subclone lanes were stained with 600 μl of conditioned culture supernatant.

FIG. 13 depicts the results of whole human serum profiled on an anionic Q10 protein chip by SELDI.

FIG. 14 is a photograph of a pseudogel showing the differentially expressed protein peaks identified in 13 T2D and 16 normal human serum samples. For the M/Z 15.2 kD marker, the average peak intensity for T2D samples was 2.6, while for normal samples, the average peak intensity was 22.2. The difference between the two samples was about 9-fold. For the M/Z 14.8 kD marker, the average intensity for T2D samples was 4.4, and the average intensity for normal samples was 3.3. The relative intensity ratio was 1.47.

FIG. 15 is a photograph of a pseudogel showing the differentially expressed protein peaks identified in 13 T2D and 16 normal human serum samples. The average peak intensity for T2D samples was 118, while for normal samples, the average peak intensity was 182. The ratio of relative intensity was 0.65. Each dot represents the intensity of the protein peak measured in individual samples.

FIG. 16A is a graph depicting differential albumin profiling in samples obtained from obese T2D subjects (Dr. Cheatham's samples) vs. non-obese T2D subjects (Dr. Dankner's samples).

FIG. 16B depicts a Western blot of proteins identified using polyclonal anti-D3 antibodies and the relative abundance of the protein by quantification of band intensity.

FIGS. 17A and 17B are graphical representations of ELISA reactivity of CDs-HSD and CDr-HSD specific hybridoma colonies, as measured by absorbance at O.D. 450 nm.

FIGS. 18A, 18B, and 18C are photographs of Western blots depicting the reactivity of the CDs-HSD and CDr-HSD specific hybridoma clones P2-10-B8-KA8, P1-14-A2-E-H8, P2-4-H5-K-B4, P1-20-B7-F-C1, P2-13-A9-P-A8, and P1-5-F11-XF5.

FIG. 19 is a photograph of a Coomassie-stained SDS-polyacrylamide gel following immunoprecipitation with the specific hybridoma clones derived from CDs-HSD and CDr-HSD.

FIGS. 20A and 20B are screenshots of an MS spectrum analysis of the lower bands excised from the SDS-PAGE gel in FIG. 18. A positive identification of the lower band as calnexin was made.

FIG. 21 is a scatter plot of the 137 differentially expressed genes in Cohen Type 2 Diabetes rat pancreas. Both upregulated and downregulated genes are shown on the plot.

FIG. 22A depicts Gene Tree microarray analysis of 12,729 genes present in Cohen Type 2 Diabetes rat pancreas.

FIG. 22B depicts Gene Tree microarray analysis of the 820 genes that were found to have 2-fold changes in expression, and the 137 genes shown to have 3-fold changes in expression in Cohen Type 2 Diabetes rat pancreas.

FIG. 22C depicts the Sets 1-5 of the 137 genes exhibiting 3-fold changes in expression, as classified by K-mean clustering.

### DETAILED DESCRIPTION OF THE INVENTION

The present invention relates to the identification of biomarkers associated with subjects having Diabetes or a pre-diabetic condition, or who are pre-disposed to developing Diabetes or a pre-diabetic condition. Accordingly, the present invention features diagnostic and prognostic methods for identifying subjects who are pre-disposed to developing Diabetes or a pre-diabetic condition, including those subjects who are asymptomatic for Diabetes or a pre-diabetic condition by detection of the biomarkers disclosed herein. The biomarkers and methods of the present invention allow one of skill in the art to identify, diagnose, or otherwise assess those subjects who do not exhibit any symptoms of Diabetes or a pre-diabetic condition, but who nonetheless may be at risk for developing Diabetes or experiencing symptoms characteristic of a pre-diabetic condition. The biomarkers can also be used advantageously to identify subjects having or at risk for developing complications relating to Type 2 Diabetes. These biomarkers are also useful for monitoring subjects undergoing treatments and therapies for Diabetes or pre-diabetic conditions, and for selecting therapies and treatments that would be effective in subjects having Diabetes or a pre-diabetic condition, wherein selection and use of such treatments and therapies slow the progression of Diabetes or pre-diabetic conditions, or substantially delay or prevent its onset. The biomarkers of the present invention can be in the form of a pharmaceutical composition used to treat subjects having type 2 Diabetes or related conditions.

As used herein, “a,” an” and “the” include singular and plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

The term “analyte” as used herein can mean any substance to be measured and can encompass electrolytes and elements, such as calcium. Finally, biomarkers can also refer to non-analyte physiological markers of health status encompassing other clinical characteristics such as, without limitation, age, ethnicity, diastolic and systolic blood pressure, body-mass index, and resting heart rate.

The term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques.

As used herein, the term “antigen binding region” refers to that portion of an antibody molecule which contains the amino acid residues that bind and interact with an antigen and confer on the antibody its specificity and affinity for the antigen. The antibody region includes the “framework” amino acid residues necessary to maintain the proper conformation of the antigen-binding residues.

An “antigen” is a molecule or a portion of a molecule capable of being bound by an antibody which is additionally capable of inducing an animal to produce antibody capable of binding to an epitope of that antigen. An antigen can have one or more than one epitope. The specific reaction referred to above is meant to indicate that the antigen will react, in a highly selective manner, with its corresponding antibody and not with the multitude of other antibodies which can be evoked by other antigens. Preferred antigens that bind antibodies, fragments and regions of antibodies of the present invention include at least one, preferably two, three, four, five, six, seven, eight, nine, ten or more amino acid residues of SEQ ID NO: 1, but can also bind to any one or more T2DBMARKERS of the invention, or metabolites thereof, such as those set forth in Table 1 herein.

The term “biomarker” in the context of the present invention encompasses, without limitation, proteins, peptides, nucleic acids, polymorphisms of proteins and nucleic acids, splice variants, fragments of proteins or nucleic acids, elements, metabolites, and other analytes. Biomarkers can also include mutated proteins or mutated nucleic acids.

“Complications related to type 2 Diabetes” or “complications related to a pre-diabetic condition” can include, without limitation, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof.

“Diabetes Mellitus” in the context of the present invention encompasses Type 1 Diabetes, both autoimmune and idiopathic and Type 2 Diabetes (together, “Diabetes”). The World Health Organization defines the diagnostic value of fasting plasma glucose concentration to 7.0 mmol/l (126 mg/dl) and above for Diabetes Mellitus (whole blood 6.1 mmol/l or 110 mg/dl), or 2-hour glucose level ≧11.1 mmol/L (≧200 mg/dL). Other values suggestive of or indicating high risk for Diabetes Mellitus include elevated arterial pressure ≧140/90 mm Hg; elevated plasma triglycerides (≧1.7 mmol/L; 150 mg/dL) and/or low HDL-cholesterol (<0.9 mmol/L, 35 mg/dl for men; <1.0 mmol/L, 39 mg/dL women); central obesity (males: waist to hip ratio >0.90; females: waist to hip ratio >0.85) and/or body mass index exceeding 30 kg/m²; microalbuminuria, where the urinary albumin excretion rate ≧20 μg/min or albumin:creatinine ratio ≧30 mg/g).

The term “epitope” is meant to refer to that portion of any molecule capable of being recognized by and bound by an antibody at one or more of the Ab's antigen binding regions. Epitopes usually consist of chemically active surface groupings of molecules such as amino acids or sugar side chains and have specific three dimensional structural characteristics as well as specific charge characteristics. An epitope can comprise the antibody binding region of any one or more of T2DBMARKERS disclosed herein, or a metabolite thereof. An epitope can also comprise at least one, preferably two, three, four, five, six, seven, eight, nine, ten or more amino acid residues of SEQ ID NO: 1. The amino acid residues of the epitope that are recognized by the isolated antibodies of the invention need not be contiguous.

“Impaired glucose tolerance” (IGT) is defined as having a blood glucose level that is higher than normal, but not high enough to be classified as Diabetes Mellitus. A subject with IGT will have two-hour glucose levels of 140 to 199 mg/dL (7.8 to 11.0 mmol) on the 75 g oral glucose tolerance test. These glucose levels are above normal but below the level that is diagnostic for Diabetes. Subjects with impaired glucose tolerance or impaired fasting glucose have a significant risk of developing Diabetes and thus are an important target group for primary prevention.

“Insulin resistance” refers to a condition in which the cells of the body become resistant to the effects of insulin, that is, the normal response to a given amount of insulin is reduced. As a result, higher levels of insulin are needed in order for insulin to exert its effects.

“Normal glucose levels” is used interchangeably with the term “normoglycemic” and refers to a fasting venous plasma glucose concentration of less than 6.1 mmol/L (110 mg/dL). Although this amount is arbitrary, such values have been observed in subjects with proven normal glucose tolerance, although some may have IGT as measured by oral glucose tolerance test (OGTT). A baseline value, index value, or reference value in the context of the present invention and defined herein can comprise, for example, “normal glucose levels.”

A “pre-diabetic condition” refers to a metabolic state that is intermediate between normal glucose homeostasis, metabolism, and states seen in frank Diabetes Mellitus. Pre-diabetic conditions include, without limitation, Metabolic Syndrome (“Syndrome X”), Impaired Glucose Tolerance (IGT), and Impaired Fasting Glycemia (IFG). IGT refers to post-prandial abnormalities of glucose regulation, while IFG refers to abnormalities that are measured in a fasting state. The World Health Organization defines values for IFG as a fasting plasma glucose concentration of 6.1 mmol/L (100 mg/dL) or greater (whole blood 5.6 mmol/L; 100 mg/dL), but less than 7.0 mmol/L (126 mg/dL)(whole blood 6.1 mmol/L; 110 mg/dL). Metabolic Syndrome according to National Cholesterol Education Program (NCEP) criteria are defined as having at least three of the following: blood pressure ≧130/85 mm Hg; fasting plasma glucose ≧6.1 mmol/L; waist circumference ≧102 cm (men) or >88 cm (women); triglycerides ≧1.7 mmol/L; and HDL cholesterol <1.0 mmol/L (men) or 1.3 mmol/L (women).

A “sample” in the context of the present invention is a biological sample isolated from a subject and can include, for example, serum, blood plasma, blood cells, endothelial cells, tissue biopsies, lymphatic fluid, pancreatic juice, ascites fluid, interstitital fluid (also known as “extracellular fluid” and encompasses the fluid found in spaces between cells, including, inter alia, gingival crevicular fluid), bone marrow, sputum, saliva, tears, or urine.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of type 2 Diabetes Mellitus or pre-diabetic conditions. A subject can be male or female. A subject can be one who has been previously diagnosed with or identified as suffering from or having type 2 Diabetes, one or more complications related to type 2 Diabetes, or a pre-diabetic condition, and optionally, but need not have already undergone treatment for the type 2 Diabetes, the one or more complications related to type 2 Diabetes, or the pre-diabetic condition. A subject can also be one who is not suffering from type 2 Diabetes or a pre-diabetic condition. A subject can also be one who has been diagnosed with or identified as suffering from type 2 Diabetes, one or more complications related to type 2 Diabetes, or a pre-diabetic condition, but who show improvements in known Diabetes risk factors as a result of receiving one or more treatments for type 2 Diabetes, one or more complications related to type 2 Diabetes, or the pre-diabetic condition. Alternatively, a subject can also be one who has not been previously diagnosed as having Diabetes, one or more complications related to type 2 Diabetes, or a pre-diabetic condition. For example, a subject can be one who exhibits one or more risk factors for Diabetes, complications related to Diabetes, or a pre-diabetic condition, or a subject who does not exhibit Diabetes risk factors, or a subject who is asymptomatic for Diabetes, one or more Diabetes-related complications, or a pre-diabetic condition. A subject can also be one who is suffering from or at risk of developing Diabetes or a pre-diabetic condition. A subject can also be one who has been diagnosed with or identified as having one or more complications related to type 2 Diabetes or a pre-diabetic condition as defined herein, or alternatively, a subject can be one who has not been previously diagnosed with or identified as having one or more complications related to type 2 Diabetes or a pre-diabetic condition.

Proteins, peptides, nucleic acids, polymorphisms, and metabolites whose levels are changed in subjects who have Diabetes or a pre-diabetic condition, or are predisposed to developing Diabetes or a pre-diabetic condition are summarized in Table 1 and are collectively referred to herein as, inter alia, “Diabetes-associated proteins”, “T2DBMARKER polypeptides”, or “T2DBMARKER proteins”. The corresponding nucleic acids encoding the polypeptides are referred to as “Diabetes-associated nucleic acids”, “Diabetes-associated genes”, “T2DBMARKER nucleic acids”, or “T2DBMARKER genes”. Unless indicated otherwise, “T2DBMARKER”, “Diabetes-associated proteins”, “Diabetes-associated nucleic acids” are meant to refer to any of the sequences disclosed herein. The corresponding metabolites of the T2DBMARKER proteins or nucleic acids can also be measured, herein referred to as “T2DBMARKER metabolites”. Calculated indices created from mathematically combining measurements of one or more, preferably two or more of the aforementioned classes of T2DBMARKERS are referred to as “T2DBMARKER indices”. Proteins, nucleic acids, polymorphisms, mutated proteins and mutated nucleic acids, metabolites, and other analytes are, as well as common physiological measurements and indices constructed from any of the preceding entities, are included in the broad category of “T2DBMARKERS”.

Five hundred and forty-eight (548) biomarkers have been identified as having altered or modified presence or concentration levels in subjects who have Diabetes, or who exhibit symptoms characteristic of a pre-diabetic condition, such as those subjects who are insulin resistant, have altered beta cell function or are at risk of developing Diabetes based upon known clinical parameters or risk factors, such as family history of Diabetes, low activity level, poor diet, excess body weight (especially around the waist), age greater than 45 years, high blood pressure, high levels of triglycerides, HDL cholesterol of less than 35, previously identified impaired glucose tolerance, previous Diabetes during pregnancy (“gestational Diabetes Mellitus”) or giving birth to a baby weighing more than nine pounds, and ethnicity.

One T2DBMARKER of interest, which has a molecular weight of about 4.2 kD and was further identified as a C-terminal fragment of a serine protease inhibitor, Serpina 3M. This marker was shown to be upregulated in CDr-RD and CDr-HSD rats. Amino acid sequencing of this fragment revealed that this fragment comprises the amino acid sequence SGRPPMIVWFNRPFLIAVSHTHGQTILFMAKVINPVGA (SEQ ID NO:1)

A T2DBMARKER “metabolite” in the context of the present invention comprises a portion of a full length polypeptide. No particular length is implied by the term “portion.” A T2DBMARKER metabolite can be less than 500 amino acids in length, e.g., less than or equal to 400, 350, 300, 250, 200, 150, 100, 75, 50, 35, 26, 25, 15, or 10 amino acids in length. An exemplary T2DBMARKER metabolite includes a peptide, which can include (in whole or in part) the sequence of SEQ ID NO:1. Preferably, the T2DBMARKER metabolite includes at least 5, 10, 15, 20, 25 or more contiguous amino acids of SEQ ID NO:1.

One or more, preferably two or more T2DBMARKERS can be detected in the practice of the present invention. For example, one (1), two (2), five (5), ten (10), fifteen (15), twenty (20), twenty-five (25), thirty (30), thirty-five (35), forty (40), forty-five (45), fifty (50), fifty-five (55), sixty (60), sixty-five (65), seventy (70), seventy-five (75), eighty (80), eighty-five (85), ninety (90), ninety-five (95), one hundred (100), one hundred and five (105), one hundred and ten (110), one hundred and fifteen (115), one hundred and twenty (120), one hundred and twenty-five (125), one hundred and thirty (130), one hundred and thirty-five (135), one hundred and forty (140), one hundred and forty-five (145), one hundred and fifty (150), one hundred and fifty-five (155), one hundred and sixty (160), one hundred and sixty-five (165), one hundred and seventy (170), one hundred and seventy-five (175), one hundred and eighty (180), one hundred and eighty-five (185), one hundred and ninety (190), one hundred and ninety-five (195), two hundred (200), two hundred and twenty-five (225), two hundred and fifty (250), two hundred and seventy-five (275), three hundred (300), three hundred and twenty-five (325), three hundred and fifty (350), three hundred and seventy-five (375), four hundred (400), four hundred and twenty-five (425), four hundred and fifty (450), four hundred and seventy-five (475), five hundred (500), five hundred and twenty-five (525), five hundred and forty (540) or more T2DBMARKERS can be detected. In some aspects, all 548 T2DBMARKERS disclosed herein can be detected. Preferred ranges from which the number of T2DBMARKERS can be detected include ranges bounded by any minimum selected from between one and 548, particularly two, five, ten, fifteen, twenty, twenty-five, thirty, forty, fifty, sixty, seventy, eighty, ninety, one hundred, one hundred and ten, one hundred and twenty, one hundred and thirty, one hundred and forty, one hundred and fifty, one hundred and seventy-five, two hundred, two hundred and twenty-five, two hundred and fifty, two hundred and seventy-five, three hundred, three hundred and twenty-five, three hundred and fifty, three hundred and seventy-five, four hundred, four hundred and twenty-five, four hundred and fifty, four hundred and seventy-five, five hundred, five hundred and twenty-five, five hundred and forty, paired with any maximum up to the total known T2DBMARKERS, particularly one, two, five, ten, twenty, and twenty-five. Particularly preferred ranges include one to two (1-2), one to five (1-5), one to ten (1-10), one to fifteen (1-15), one to twenty (1-20), one to twenty-five (1-25), one to thirty (1-30), one to thirty-five (1-35), one to forty (1-40), one to forty-five (1-45), one to fifty (1-50), one to fifty-five (1-55), one to sixty (1-60), one to sixty-five (1-65), one to seventy (1-70), one to seventy-five (1-75), one to eighty (1-80), one to eighty-five (1-85), one to ninety (1-90), one to ninety-five (1-95), one to one hundred (1-100), one to one hundred and twenty (1-120), one to one hundred and twenty-five (1-125), one to one hundred and thirty (1-130), one to one hundred and forty (1-140), one to one hundred and fifty (1-150), one to one hundred and sixty (1-160), one to one hundred and seventy-five (1-175), one to two hundred (1-200), one to two hundred and twenty-five (1-225), one to two hundred and fifty (1-250), one to two hundred and seventy-five (1-275), one to three hundred (1-300), one to three hundred and twenty-five (1-325), one to three hundred and fifty (1-350), one to three hundred and seventy-five (1-375), one to four hundred (1-400), one to four hundred and twenty-five (1-425), one to four hundred and fifty (1-450), one to four hundred and seventy-five (1-475), one to five hundred (1-500), one to five hundred and twenty-five (1-525), one to five hundred and forty (1-540), one to five hundred and forty-eight (1-548), two to five (2-5), two to ten (2-10), two to fifteen (2-15), two to twenty (2-20), two to twenty-five (2-25), two to thirty (2-30), two to thirty-five (2-35), two to forty (2-40), two to forty-five (2-45), two to fifty (2-50), two to fifty-five (2-55), two to sixty (2-60), two to sixty-five (2-65), two to seventy (2-70), two to seventy-five (2-75), two to eighty (2-80), two to eighty-five (2-85), two to ninety (2-90), two to ninety-five (2-95), two to one hundred (2-100), two to one hundred and twenty (2-120), two to one hundred and twenty-five (2-125), two to one hundred and thirty (2-130), two to one hundred and forty (2-140), two to one hundred and fifty (2-150), two to one hundred and seventy-five (2-175), two to two hundred (2-200), two to two hundred and twenty-five (2-225), two to two hundred and fifty (2-250), two to two hundred and seventy-five (2-275), two to three hundred (2-300), two to three hundred and twenty-five (2-325), two to three hundred and fifty (2-350), two to three hundred and seventy-five (2-375), two to four hundred (2-400), two to four hundred and twenty-five (2-425), two to four hundred and fifty (2-450), two to four hundred and seventy-five (2-475), two to five hundred (2-500), two to five hundred and twenty-five (2-525), two to five hundred and forty (2-540), two to five hundred and forty-eight (2-548), two to five to ten (5-10), five to fifteen (5-15), five to twenty (5-20), five to twenty-five (5-25), five to thirty (5-30), five to thirty-five (5-35), five to forty (5-40), five to forty-five (5-45), five to fifty (5-50), five to fifty-five (5-55), five to sixty (5-60), five to sixty-five (5-65), five to seventy (5-70), five to seventy-five (5-75), five to eighty (5-80), five to eighty-five (5-85), five to ninety (5-90), five to ninety-five (5-95), five to one hundred (5-100), five to one hundred and twenty (5-120), five to one hundred and twenty-five (5-125), five to one hundred and thirty (5-130), five to one hundred and forty (5-140), five to one hundred and fifty (5-150), five to one hundred and seventy-five (5-175), five to two hundred (5-200), five to two hundred and twenty-five (5-225), five to two hundred and fifty (5-250), five to two hundred and seventy-five (5-275), five to three hundred (5-300), five to three hundred and twenty-five (5-325), five to three hundred and fifty (5-350), five to three hundred and seventy-five (5-375), five to four hundred (5-400), five to four hundred and twenty-five (5-425), five to four hundred and fifty (5-450), five to four hundred and seventy-five (5-475), five to five hundred (5-500), five to five hundred and twenty-five (5-525), five to five hundred and forty (5-540), five to five hundred and forty-eight (5-548), ten to fifteen (10-15), ten to twenty (10-20), ten to twenty-five (10-25), and ten to thirty (10-30), ten to thirty-five (10-35), ten to forty (10-40), ten to forty-five (10-45), ten to fifty (10-50), ten to fifty-five (10-55), ten to sixty (10-60), ten to sixty-five (10-65), ten to seventy (10-70), ten to seventy-five (10-75), ten to eighty (10-80), ten to eighty-five (10-85), ten to ninety (10-90), ten to ninety-five (10-95), ten to one hundred (10-100), ten to one hundred and twenty (10-120), ten to one hundred and twenty-five (10-125), ten to one hundred and thirty (10-130), ten to one hundred and forty (10-140), ten to one hundred and fifty (10-150), ten to one hundred and seventy-five (10-175), ten to two hundred (10-200), ten to two hundred and twenty-five (10-225), ten to two hundred and fifty (10-250), ten to two hundred and seventy-five (10-275), ten to three hundred (10-300), ten to three hundred and twenty-five (10-325), ten to three hundred and fifty (10-350), ten to three hundred and seventy-five (10-375), ten to four hundred (10-400), ten to four hundred and twenty-five (10-425), ten to four hundred and fifty (10-450), ten to four hundred and seventy-five (10-475), ten to five hundred (10-500), ten to five hundred and twenty-five (10-525), ten to five hundred and forty (10-540), ten to five hundred and forty-eight (10-548), twenty to fifty (20-50), twenty to seventy-five (20-75), twenty to one hundred (20-100), twenty to one-hundred and twenty (20-120), twenty to one hundred and twenty-five (20-125), twenty to one hundred and thirty (20-130), twenty to one hundred and forty (20-140), twenty to one hundred and fifty (20-150), twenty to one hundred and seventy-five (20-175), twenty to two hundred (20-200), twenty to two hundred and twenty-five (20-225), twenty to two hundred and fifty (20-250), twenty to two hundred and seventy-five (20-275), twenty to three hundred (20-300), twenty to three hundred and twenty-five (20-325), twenty to three hundred and fifty (20-350), twenty to three hundred and seventy-five (20-375), twenty to four hundred (20-400), twenty to four hundred and twenty-five (20-425), twenty to four hundred and fifty (20-450), twenty to four hundred and seventy-five (20-475), twenty to five hundred (20-500), twenty to five hundred and twenty-five (20-525), twenty to five hundred and forty (20-540), twenty to five hundred and forty-eight (20-548), fifty to seventy-five (50-75), fifty to one hundred (50-100), fifty to one hundred and twenty (50-120), fifty to one hundred and twenty-five (50-125), fifty to one hundred and thirty (50-130), fifty to one hundred and forty (50-140), fifty to one hundred and fifty (50-150), fifty to one hundred and seventy-five (50-175), fifty to two hundred (50-200), fifty to two hundred and twenty-five (50-225), fifty to two hundred and fifty (50-250), fifty to two hundred and seventy-five (50-275), fifty to three hundred (50-300), fifty to three hundred and twenty-five (50-325), fifty to three hundred and fifty (50-350), fifty to three hundred and seventy-five (50-375), fifty to four hundred (50-400), fifty to four hundred and twenty-five (50-425), fifty to four hundred and fifty (50-450), fifty to four hundred and seventy-five (50-475), fifty to five hundred (50-500), fifty to five hundred and twenty-five (50-525), fifty to five hundred and forty (50-540), fifty to five hundred and forty-eight (50-548), one hundred to one hundred and twenty-five (100-125), one hundred to one hundred and fifty (100-150), one hundred to one hundred and seventy-five (100-175), one hundred to two hundred (100-200), one hundred to two hundred and twenty-five (100-225), one hundred to two hundred and fifty (100-250), one hundred to two hundred and seventy-five (100-275), one hundred to three hundred (100-300), one hundred to three hundred and twenty-five (100-325), one hundred to three hundred and fifty (100-350), one hundred to three hundred and seventy-five (100-375), one hundred to four hundred (100-400), one hundred to four hundred and twenty-five (50-425), one hundred to four hundred and fifty (100-450), one hundred to four hundred and seventy-five (100-475), one hundred to five hundred (100-500), one hundred to five hundred and twenty-five (100-525), one hundred to five hundred and forty (100-540), one hundred to five hundred and forty-eight (100-548), one hundred and twenty-five to one hundred and fifty (125-150), one hundred and twenty-five to one hundred and seventy-five (125-175), one hundred and twenty-five to two hundred (125-200), one hundred and twenty-five to two hundred and twenty-five (125-225), one hundred and twenty-five to two hundred and fifty (125-250), one hundred and twenty-five to two hundred and seventy-five (125-275), one hundred and twenty-five to three hundred (125-300), one hundred and twenty-five to three hundred and twenty-five (125-325), one hundred and twenty-five to three hundred and fifty (125-350), one hundred and twenty-five to three hundred and seventy-five (125-375), one hundred and twenty-five to four hundred (125-400), one hundred and twenty-five to four hundred and twenty-five (125-425), one hundred and twenty-five to four hundred and fifty (125-450), one hundred and twenty-five to four hundred and seventy-five (125-475), one hundred and twenty-five to five hundred (125-500), one hundred and twenty-five to five hundred and twenty-five (125-525), one hundred and twenty-five to five hundred and forty (125-540), one hundred and twenty-five to five hundred and forty-eight (125-548), one hundred and fifty to one hundred and seventy-five (150-175), one hundred and fifty to two hundred (150-200), one hundred and fifty to two hundred and twenty-five (150-225), one hundred and fifty to two hundred and fifty (150-250), one hundred and fifty to two hundred and seventy-five (150-275), one hundred and fifty to three hundred (150-300), one hundred and fifty to three hundred and twenty-five (150-325), one hundred and fifty to three hundred and fifty (150-350), one hundred and fifty to three hundred and seventy-five (150-375), one hundred and fifty to four hundred (150-400), one hundred and fifty to four hundred and twenty-five (150-425), one hundred and fifty to four hundred and fifty (150-450), one hundred and fifty to four hundred and seventy-five (150-475), one hundred and fifty to five hundred (150-500), one hundred and fifty to five hundred and twenty-five (150-525), one hundred and fifty to five hundred and forty (150-540), and one hundred and fifty to five hundred and forty-eight (150-548).

### Diagnostic and Prognostic Methods

The risk of developing Diabetes, one or more complications related to Diabetes, or Pre-diabetic condition can be detected by examining an “effective amount” of T2DBMARKER proteins, peptides, nucleic acids, polymorphisms, metabolites, and other analytes in a test sample (e.g., a subject derived sample) and comparing the effective amounts to reference or index values. An “effective amount” can be the total amount or levels of T2DBMARKERS that are detected in a sample, or it can be a “normalized” amount, e.g., the difference between T2DBMARKERS detected in a sample and background noise. Normalization methods and normalized values will differ depending on the method of detection. Preferably, mathematical algorithms can be used to combine information from results of multiple individual T2DBMARKERS into a single measurement or index. Subjects identified as having an increased risk of Diabetes, one or more complications related to Diabetes, one or more complications related to Diabetes, or a pre-diabetic condition can optionally be selected to receive treatment regimens, such as administration of prophylactic or therapeutic compounds such as “diabetes-modulating agents” as defined herein, or implementation of exercise regimens or dietary supplements to prevent or delay the onset of Diabetes, one or more complications related to Diabetes, or a pre-diabetic condition. A sample isolated from the subject can comprise, for example, blood, plasma, blood cells, endothelial cells, tissue biopsies, lymphatic fluid, pancreatic juice, serum, bone marrow, ascites fluid, interstitial fluid (including, for example, gingival crevicular fluid), urine, sputum, saliva, tears, or other bodily fluids.

The amount of the T2DBMARKER protein, peptide, nucleic acid, polymorphism, metabolite, or other analyte can be measured in a test sample and compared to the normal control level. The term “normal control level”, means the level of one or more T2DBMARKER proteins, nucleic acids, polymorphisms, metabolites, or other analytes, or T2DBMARKER indices, typically found in a subject not suffering from Diabetes, one or more complications related to Diabetes, or a pre-diabetic condition and not likely to have Diabetes, one or more complications related to Diabetes, or a pre-diabetic condition, e.g., relative to samples collected from longitudinal studies of young subjects who were monitored until advanced age and were found not to develop Diabetes, one or more complications related to Diabetes, or a pre-diabetic condition. The “normal control level” can encompass values obtained from a subject having “normal glucose levels” or “normoglycemic levels” as defined herein. Alternatively, the normal control level can mean the level of one or more T2DBMARKER protein, peptide, nucleic acid, polymorphism, metabolite, or other analyte typically found in a subject suffering from Diabetes, one or more complications related to Diabetes, or a pre-diabetic condition. The normal control level can be a range or an index. Alternatively, the normal control level can be a database of patterns from previously tested subjects. A change in the level in the subject-derived sample of one or more T2DBMARKER protein, nucleic acid, polymorphism, metabolite, or other analyte compared to the normal control level can indicate that the subject is suffering from or is at risk of developing Diabetes, one or more complications related to Diabetes, or a pre-diabetic condition. In contrast, when the methods are applied prophylactically, a similar level compared to the normal control level in the subject-derived sample of one or more T2DBMARKER proteins, nucleic acids, polymorphisms, metabolites, or other analytes can indicate that the subject is not suffering from, is not at risk or is at low risk of developing Diabetes, one or more complications related to Diabetes, or a pre-diabetic condition.

A reference value can refer to values obtained from a control subject or population whose diabetic state is known (i.e., has been diagnosed with or identified as suffering from type 2 Diabetes, one or more complications related to Diabetes, or a pre-diabetic condition, or has not been diagnosed with or identified as suffering from type 2 Diabetes, one or more complications related to Diabetes, or a pre-diabetic condition) or can be an index value or baseline value. The reference sample or index value or baseline value may be taken or derived from one or more subjects who have been exposed to the treatment, or may be taken or derived from one or more subjects who are at low risk of developing Diabetes, one or more complications related to Diabetes, or a pre-diabetic condition, or may be taken or derived from subjects who have shown improvements in Diabetes risk factors as a result of exposure to treatment. Alternatively, the reference sample or index value or baseline value may be taken or derived from one or more subjects who have not been exposed to the treatment. For example, samples may be collected from subjects who have received initial treatment for Diabetes, one or more complications related to Diabetes, or a pre-diabetic condition and subsequent treatment for Diabetes, one or more complications related to Diabetes, or a pre-diabetic condition to monitor the progress of the treatment. A reference value can also comprise a value derived from risk prediction algorithms or computed indices from population studies such as those disclosed herein. A reference value can also be a value derived from a subject previously identified as having one or more complications related to type 2 Diabetes or a pre-diabetic condition, or alternatively, a value derived from a subject who has not developed complications, or has not been previously diagnosed with or identified as having complications relating to type 2 Diabetes or a pre-diabetic condition. A reference value can also comprise a value corresponding to the normal control level or derived from one or more subjects having “normal glucose levels” as defined herein.

Differences in the level or amounts (which can be an “effective amount”) of T2DBMARKERS measured by the methods of the present invention can comprise increases or decreases in the level or amounts of T2DBMARKERS. The increase or decrease in the amounts of T2DBMARKERS relative to a reference value can be indicative of progression of type 2 Diabetes or a pre-diabetic condition, delay, progression, development, or amelioration of complications related to type 2 Diabetes or a pre-diabetic condition, an increase or decrease in the risk of developing type 2 Diabetes or a pre-diabetic condition, or complications relating thereto. The increase or decrease can be indicative of the success of one or more treatment regimens for type 2 Diabetes, one or more complications related to Diabetes, or a pre-diabetic condition, or can indicate improvements or regression of Diabetes risk factors. The increase or decrease can be, for example, at least 5%, at least 10%, at least 15%, at least 20%, at least 25%, at least 30%, at least 35%, at least 40%, at least 45%, or at least 50% of the reference value or normal control level.

The difference in the level (or amounts) of T2DBMARKERS is preferably statistically significant. By “statistically significant”, it is meant that the alteration is greater than what might be expected to happen by chance alone. Statistical significance can be determined by any method known in the art. For example, statistical significance can be determined by p-value. The p-value is a measure of probability that a difference between groups during an experiment happened by chance. (P(z>zobserved)). For example, a p-value of 0.01 means that there is a 1 in 100 chance the result occurred by chance. The lower the p-value, the more likely it is that the difference between groups was caused by treatment. An alteration is statistically significant if the p-value is at least 0.05. Preferably, the p-value is 0.04, 0.03, 0.02, 0.01, 0.005, 0.001 or less. As noted below, and without any limitation of the invention, achieving statistical significance generally but not always requires that combinations of several T2DBMARKERS be used together in panels and combined with mathematical algorithms in order to achieve a statistically significant T2DBMARKER index.

The “diagnostic accuracy” of a test, assay, or method concerns the ability of the test, assay, or method to distinguish between subjects having Diabetes, one or more complications related to Diabetes, or a pre-diabetic condition, or at risk for Diabetes, one or more complications related to Diabetes, or a pre-diabetic condition is based on whether the subjects have a “clinically significant presence” or a “clinically significant alteration” in the levels of one or more T2DBMARKERS. By “clinically significant presence” or “clinically significant alteration”, it is meant that the presence of the T2DBMARKER (e.g., mass, such as milligrams, nanograms, or mass per volume, such as milligrams per deciliter or copy number of a transcript per unit volume) or an alteration in the presence of the T2DBMARKER in the subject (typically in a sample from the subject) is higher than the predetermined cut-off point (or threshold value) for that T2DBMARKER and therefore indicates that the subject has Diabetes, one or more complications related to Diabetes, or a pre-diabetic condition for which the sufficiently high presence of that protein, peptide, nucleic acid, polymorphism, metabolite or analyte is a marker.

The present invention may be used to make categorical or continuous measurements of the risk of conversion to Type 2 Diabetes, thus diagnosing a category of subjects defined as pre-Diabetic.

In the categorical scenario, the methods of the present invention can be used to discriminate between normal and pre-diabetic condition subject cohorts. In this categorical use of the invention, the terms “high degree of diagnostic accuracy” and “very high degree of diagnostic accuracy” refer to the test or assay for that T2DBMARKER (or T2DBMARKER index; wherein T2DBMARKER value encompasses any individual measurement whether from a single T2DBMARKER or derived from an index of T2DBMARKERS) with the predetermined cut-off point correctly (accurately) indicating the presence or absence of a pre-diabetic condition. A perfect test would have perfect accuracy. Thus, for subjects who have a pre-diabetic condition, the test would indicate only positive test results and would not report any of those subjects as being “negative” (there would be no “false negatives”). In other words, the “sensitivity” of the test (the true positive rate) would be 100%. On the other hand, for subjects who did not have a pre-diabetic condition, the test would indicate only negative test results and would not report any of those subjects as being “positive” (there would be no “false positives”). In other words, the “specificity” (the true negative rate) would be 100%. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which discusses specificity, sensitivity, and positive and negative predictive values of a test, e.g., a clinical diagnostic test. In other embodiments, the present invention may be used to discriminate a pre-diabetic condition from Diabetes, or Diabetes from Normal. Such use may require different subsets of T2DBMARKERS(out of the total T2DBMARKERS as disclosed in Table 1), mathematical algorithm, and/or cut-off point, but be subject to the same aforementioned measurements of diagnostic accuracy for the intended use.

In the categorical diagnosis of a disease, changing the cut point or threshold value of a test (or assay) usually changes the sensitivity and specificity, but in a qualitatively inverse relationship. For example, if the cut point is lowered, more subjects in the population tested will typically have test results over the cut point or threshold value. Because subjects who have test results above the cut point are reported as having the disease, condition, or syndrome for which the test is conducted, lowering the cut point will cause more subjects to be reported as having positive results (e.g., that they have Diabetes, one or more complications related to Diabetes, or a pre-diabetic condition). Thus, a higher proportion of those who have Diabetes, one or more complications related to Diabetes, or a pre-diabetic condition will be indicated by the test to have it. Accordingly, the sensitivity (true positive rate) of the test will be increased. However, at the same time, there will be more false positives because more people who do not have the disease, condition, or syndrome (e.g., people who are truly “negative”) will be indicated by the test to have T2DBMARKER values above the cut point and therefore to be reported as positive (e.g., to have the disease, condition, or syndrome) rather than being correctly indicated by the test to be negative. Accordingly, the specificity (true negative rate) of the test will be decreased. Similarly, raising the cut point will tend to decrease the sensitivity and increase the specificity. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points.

There is, however, an indicator that allows representation of the sensitivity and specificity of a test, assay, or method over the entire range of test (or assay) cut points with just a single value. That indicator is derived from a Receiver Operating Characteristics (“ROC”) curve for the test, assay, or method in question. See, e.g., Shultz, “Clinical Interpretation Of Laboratory Procedures,” chapter 14 in Teitz, Fundamentals of Clinical Chemistry, Burtis and Ashwood (eds.), 4^{th} edition 1996, W.B. Saunders Company, pages 192-199; and Zweig et al., “ROC Curve Analysis: An Example Showing The Relationships Among Serum Lipid And Apolipoprotein Concentrations In Identifying Subjects With Coronory Artery Disease,” Clin. Chem., 1992, 38(8): 1425-1428.

An ROC curve is an x-y plot of sensitivity on the y-axis, on a scale of zero to one (e.g., 100%), against a value equal to one minus specificity on the x-axis, on a scale of zero to one (e.g., 100%). In other words, it is a plot of the true positive rate against the false positive rate for that test, assay, or method. To construct the ROC curve for the test, assay, or method in question, subjects can be assessed using a perfectly accurate or “gold standard” method that is independent of the test, assay, or method in question to determine whether the subjects are truly positive or negative for the disease, condition, or syndrome (for example, coronary angiography is a gold standard test for the presence of coronary atherosclerosis). The subjects can also be tested using the test, assay, or method in question, and for varying cut points, the subjects are reported as being positive or negative according to the test, assay, or method. The sensitivity (true positive rate) and the value equal to one minus the specificity (which value equals the false positive rate) are determined for each cut point, and each pair of x-y values is plotted as a single point on the x-y diagram. The “curve” connecting those points is the ROC curve.

The ROC curve is often used in order to determine the optimal single clinical cut-off or treatment threshold value where sensitivity and specificity are maximized; such a situation represents the point on the ROC curve which describes the upper left corner of the single largest rectangle which can be drawn under the curve.

The total area under the curve (“AUC”) is the indicator that allows representation of the sensitivity and specificity of a test, assay, or method over the entire range of cut points with just a single value. The maximum AUC is one (a perfect test) and the minimum area is one half (e.g. the area where there is no discrimination of normal versus disease). The closer the AUC is to one, the better is the accuracy of the test. It should be noted that implicit in all ROC and AUC is the definition of the disease and the post-test time horizon of interest.

By a “high degree of diagnostic accuracy”, it is meant a test or assay in which the AUC (area under the ROC curve for the test or assay) is at least 0.70, desirably at least 0.75, more desirably at least 0.80, preferably at least 0.85, more preferably at least 0.90, and most preferably at least 0.95.

By a “very high degree of diagnostic accuracy”, it is meant a test or assay in which the AUC (area under the ROC curve for the test or assay) is at least 0.80, desirably at least 0.85, more desirably at least 0.875, preferably at least 0.90, more preferably at least 0.925, and most preferably at least 0.95.

Alternatively, in low disease prevalence tested populations (defined as those with less than 1% rate of occurrences per annum), ROC and AUC can be misleading as to the clinical utility of a test, and absolute and relative risk ratios as defined elsewhere in this disclosure can be employed to determine the degree of diagnostic accuracy. Populations of subjects to be tested can also be categorized into quartiles, where the top quartile (25% of the population) comprises the group of subjects with the highest relative risk for developing or suffering from Diabetes, one or more complications related to Diabetes, or a pre-diabetic condition and the bottom quartile comprising the group of subjects having the lowest relative risk for developing Diabetes, one or more complications related to Diabetes, or a pre-diabetic condition. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a very high degree of diagnostic accuracy. Nonetheless, values derived from tests or assays having only 1.2 to 2.5 times the relative risk for each quartile remain clinically useful are widely used as risk factors for a disease; such is the case with insulin levels or blood glucose levels with respect to their prediction of future type 2 Diabetes.

The predictive value of any test depends on the sensitivity and specificity of the test, and on the prevalence of the condition in the population being tested. This notion, based on Bayes' theorem, provides that the greater the likelihood that the condition being screened for is present in a subject or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative. By defining the degree of diagnostic accuracy, i.e., cut points on a ROC curve, defining an acceptable AUC value, and determining the acceptable ranges in relative concentration of what constitutes an effective amount of the T2DBMARKERS of the invention allows one of skill in the art to use the T2DBMARKERS to diagnose or identify subjects with a pre-determined level of predictability.

Alternative methods of determining diagnostic accuracy must be used with continuous measurements of risk, which are commonly used when a disease category or risk category (such as a pre-diabetic condition) has not yet been clearly defined by the relevant medical societies and practice of medicine.

“Risk” in the context of the present invention can mean “absolute” risk, which refers to that percentage probability that an event will occur over a specific time period. Absolute risk can be measured with reference to either actual observation post-measurement for the relevant time cohort, or with reference to index values developed from statistically valid historical cohorts that have been followed for the relevant time period. “Relative” risk refers to the ratio of absolute risks of a subject's risk compared either to low risk cohorts or average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. Alternative continuous measures which may be assessed in the context of the present invention include time to Diabetes conversion and therapeutic Diabetes conversion risk reduction ratios.

For such continuous measures, measures of diagnostic accuracy for a calculated index are typically based on linear regression curve fits between the predicted continuous value and the actual observed values (or historical index calculated value) and utilize measures such as R squared, p values and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health (Redwood City, Calif.).

The ultimate determinant and gold standard of true risk conversion to Diabetes is actual conversions within a sufficiently large population and observed over a particular length of time. However, this is problematic, as it is necessarily a retrospective point of view, coming after any opportunity for preventive interventions. As a result, subjects suffering from or at risk of developing Diabetes, one or more complications related to Diabetes, or a pre-diabetic condition are commonly diagnosed or identified by methods known in the art, and future risk is estimated based on historical experience and registry studies. Such methods include, but are not limited to, measurement of systolic and diastolic blood pressure, measurements of body mass index, in vitro determination of total cholesterol, LDL, HDL, insulin, and glucose levels from blood samples, oral glucose tolerance tests, stress tests, measurement of human serum C-reactive protein (hsCRP), electrocardiogram (ECG), c-peptide levels, anti-insulin antibodies, anti-beta cell-antibodies, and glycosylated hemoglobin (HbA_{1c}). Additionally, any of the aforementioned methods can be used separately or in combination to assess if a subject has shown an “improvement in Diabetes risk factors.” Such improvements include, without limitation, a reduction in body mass index (BMI), a reduction in blood glucose levels, an increase in HDL levels, a reduction in systolic and/or diastolic blood pressure, an increase in insulin levels, or combinations thereof.

The oral glucose tolerance test (OGTT) is principally used for diagnosis of Diabetes Mellitus or pre-diabetic conditions when blood glucose levels are equivocal, during pregnancy, or in epidemiological studies (Definition, Diagnosis and Classification of Diabetes Mellitus and its Complications, Part 1, World Health Organization, 1999). The OGTT should be administered in the morning after at least 3 days of unrestricted diet (greater than 150 g of carbohydrate daily) and usual physical activity. A reasonable (30-50 g) carbohydrate-containing meal should be consumed on the evening before the test. The test should be preceded by an overnight fast of 8-14 hours, during which water may be consumed. After collection of the fasting blood sample, the subject should drink 75 g of anhydrous glucose or 82.5 g of glucose monohydrate in 250-300 ml of water over the course of 5 minutes. For children, the test load should be 1.75 g of glucose per kg body weight up to a total of 75 g of glucose. Timing of the test is from the beginning of the drink. Blood samples must be collected 2 hours after the test load. As previously noted, a diagnosis of impaired glucose tolerance (IGT) has been noted as being only 50% sensitive, with a >10% false positive rate, for a 7.5 year conversion to Diabetes when used at the WHO cut-off points. This is a significant problem for the clinical utility of the test, as even relatively high risk ethnic groups have only a 10% rate of conversion to Diabetes over such a period unless otherwise enriched by other risk factors; in an unselected general population, the rate of conversion over such periods is typically estimated at 5-6%, or less than 1% per annum.

Other methods of measuring glucose in blood include reductiometric methods known in the art such as, but not limited to, the Somogyi-Nelson method, methods using hexokinase and glucose dehydrogenase, immobilized glucose oxidase electrodes, the o-toluidine method, the ferricyanide method and the neocuprine autoanalyzer method. Whole blood glucose values are usually about 15% lower than corresponding plasma values in patients with a normal hematocrit reading, and arterial values are generally about 7% higher than corresponding venous values. Subjects taking insulin are frequently requested to build up a “glycemic profile” by self-measurement of blood glucose at specific times of the day. A “7-point profile” is useful, with samples taken before and 90 minutes after each meal, and just before going to bed.

A subject suffering from or at risk of developing Diabetes, one or more complications related to Diabetes, or a pre-diabetic condition may also be suffering from or at risk of developing cardiovascular disease, hypertension or obesity. Type 2 Diabetes in particular and cardiovascular disease have many risk factors in common, and many of these risk factors are highly correlated with one another. The relationships among these risk factors may be attributable to a small number of physiological phenomena, perhaps even a single phenomenon. In addition to detecting levels of one or more T2DBMARKERS of the invention, subjects suffering from or at risk of developing Diabetes, one or more complications related to Diabetes, cardiovascular disease, hypertension or obesity can be identified by methods known in the art. For example, Diabetes is frequently diagnosed by measuring fasting blood glucose levels or insulin. Normal adult glucose levels are 60-126 mg/dl. Normal insulin levels are 7 mU/ml±3mU. Hypertension is diagnosed by a blood pressure consistently at or above 140/90. Risk of cardiovascular disease can also be diagnosed by measuring cholesterol levels. For example, LDL cholesterol above 137 or total cholesterol above 200 is indicative of a heightened risk of cardiovascular disease. Obesity is diagnosed for example, by body mass index. Body mass index (BMI) is measured (kg/m² (or lb/in²×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e. fat mass impedes current), estimates % fat) can be measured. The parameters for normal, overweight, or obese individuals is as follows: Underweight: BMI <18.5; Normal: BMI 18.5 to 24.9; Overweight: BMI=25 to 29.9. Overweight individuals are characterized as having a waist circumference of >94 cm for men or >80 cm for women and waist to hip ratios of ≧0.95 in men and ≧0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of ≧35. Because of the interrelationship between Diabetes and cardiovascular disease, some or all of the individual T2DBMARKERS and T2DBMARKER expression profiles of the present invention may overlap or be encompassed by biomarkers of cardiovascular disease, and indeed may be useful in the diagnosis of the risk of cardiovascular disease.

Risk prediction for Diabetes Mellitus, one or more complications related to Diabetes, or a pre-diabetic condition can also encompass risk prediction algorithms and computed indices that assess and estimate a subject's absolute risk for developing Diabetes, one or more complications related to Diabetes, or a pre-diabetic condition with reference to a historical cohort. Risk assessment using such predictive mathematical algorithms and computed indices has increasingly been incorporated into guidelines for diagnostic testing and treatment, and encompass indices obtained from and validated with, inter alia, multi-stage, stratified samples from a representative population. A plurality of conventional Diabetes risk factors are incorporated into predictive models. A notable example of such algorithms include the Framingham Heart Study (Kannel, W. B., et al, (1976) Am. J. Cardiol. 38: 46-51) and modifications of the Framingham Study, such as the National Cholesterol Education Program Expert Panel on Detection, Evaluation, and Treatment of High Blood Cholesterol in Adults (Adult Treatment Panel III), also know as NCEP/ATP III, which incorporates a patient's age, total cholesterol concentration, HDL cholesterol concentration, smoking status, and systolic blood pressure to estimate a person's 10-year risk of developing cardiovascular disease, which is commonly found in subjects suffering from or at risk for developing Diabetes Mellitus, one or more complications related to Diabetes, or a pre-diabetic condition. The Framingham algorithm has been found to be modestly predictive of the risk for developing Diabetes Mellitus, or a pre-diabetic condition.

Other Diabetes risk prediction algorithms include, without limitation, the San Antonio Heart Study (Stem, M. P. et al, (1984) Am. J. Epidemiol. 120: 834-851; Stem, M. P. et al, (1993) Diabetes 42: 706-714; Burke, J. P. et al, (1999) Arch. Intern. Med. 159: 1450-1456), Archimedes (Eddy, D. M. and Schlessinger, L. (2003) Diabetes Care 26(11): 3093-3101; Eddy, D. M. and Schlessinger, L. (2003) Diabetes Care 26(11): 3102-3110), the Finnish-based Diabetes Risk Score (Lindstrom, J. and Tuomilehto, J. (2003) Diabetes Care 26(3): 725-731), and the Ely Study (Griffin, S. J. et al, (2000) Diabetes Metab. Res. Rev. 16: 164-171), the contents of which are expressly incorporated herein by reference.

Archimedes is a mathematical model of Diabetes that simulates the disease state person-by-person, object-by-object and comprises biological details that are continuous in reality, such as the pertinent organ systems, more than 50 continuously interacting biological variables, and the major symptoms, tests, treatments, and outcomes commonly associated with Diabetes.

Archimedes includes many diseases simultaneously and interactively in a single integrated physiology, enabling it to address features such as co-morbidities, syndromes, treatments and other multiple effects. The Archimedes model includes Diabetes and its complications, such as coronary artery disease, congestive heart failure, and asthma. The model is written in differential equations, using object-oriented programming and a construct called “features”. The model comprises the anatomy of a subject (all simulated subjects have organs, such as hearts, livers, pancreases, gastrointestinal tracts, fat, muscles, kidneys, eyes, limbs, circulatory systems, brains, skin, and peripheral nervous systems), the “features” that determine the course of the disease and representing real physical phenomena (e.g., the number of milligrams of glucose in a deciliter of plasma, behavioral phenomena, or conceptual phenomena (e.g., the “progression” of disease), risk factors, incidence, and progression of the disease, glucose metabolism, signs and tests, diagnosis, symptoms, health outcomes of glucose metabolism, treatments, complications, deaths from Diabetes and its complications, deaths from other causes, care processes, and medical system resources. For a typical application of the model, there are thousands of simulated subjects, each with a simulated anatomy and physiology, who will get simulated diseases, can seek care at simulated health care facilities, will be seen by simulated health care personnel in simulated facilities, will be given simulated tests and treatments, and will have simulated outcomes. As in reality, each of the simulated patients is different, with different characteristics, physiologies, behaviors, and responses to treatments, all designed to match the individual variations seen in reality.

The model is built by development of a non-quantitative or conceptual description of the pertinent biology and pathology—the variables and relationships—as best they are understood with current information. Studies are then identified that pertain to the variables and relationships, and typically comprise basic research, epidemiological, and clinical studies that experts in the field identify as the foundations of their own understanding of the disease. That information is used to develop differential equations that relate the variables. The development of any particular equation in the Archimedes model involves finding the form and coefficients that best fit the available information about the variables, after which the equations are programmed into an object-oriented language. This is followed by a series of exercises in which the parts of the model are tested and debugged, first one at a time, and then in appropriate combinations, using inputs that have known outputs. The entire model can then be used to simulate a complex trial, which demonstrates not only the individual parts of the model, but also the connections between all the parts. The Archimedes calculations are performed using distributed computing techniques. Archimedes has been validated as a realistic representation of the anatomy, pathophysiology, treatments and outcomes pertinent to Diabetes and its complications (Eddy, D. M. and Schlessinger, L. (2003) Diabetes Care 26(11) 3102-3110).

The Finland-based Diabetes Risk Score is designed as a screening tool for identifying high-risk subjects in the population and for increasing awareness of the modifiable risk factors and healthy lifestyle. The Diabetes Risk Score was determined from a random population sample of 35- to 64-year old Finnish men and women with no anti-diabetic drug treatment at baseline, and followed for 10 years. Multivariate logistic regression model coefficients were used to assign each variable category a score. The Diabetes Risk Score comprises the sum of these individual scores and validated in an independent population survey performed in 1992 with a prospective follow-up for 5 years. Age, BMI, waist circumference, history of anti-hypertensive drug treatment and high blood glucose, physical activity, and daily consumption of fruits, berries, or vegetables were selected as categorical variables.

The Finland-based Diabetes Risk Score values are derived from the coefficients of the logistic model by classifying them into five categories. The estimated probability (p) of drug-treated Diabetes over a 10-year span of time for any combination of risk factors can be calculated from the following coefficients:

$$p ( Diabetes ) = \frac{{e}^{( {β}_{0} + {β}_{1 \times 1} + {β}_{2 \times 2} + … )}}{1 + {e}^{( {β}_{0} + {β}_{1 \times 1} + {β}_{2 \times 2} + … )}}$$

where β₀ is the intercept and β₁, β₂, and so on represent the regression coefficients of the various categories of the risk factors x₁, x₂, and so on.

The sensitivity relates to the probability that the test is positive for subjects who will get drug-treated Diabetes in the future and the specificity reflects the probability that the test is negative for subjects without drug-treated Diabetes. The sensitivity and the specificity with 95% confidence interval (CI) were calculated for each Diabetes Risk Score level in differentiating the subjects who developed drug-treated Diabetes from those who did not. ROC curves were plotted for the Diabetes Risk score, the sensitivity was plotted on the y-axis and the false-positive rate (1-specificity) was plotted on the x-axis. The more accurately discriminatory the test, the steeper the upward portion of the ROC curve, and the higher the AUC, the optimal cut point being the peak of the curve.

Statistically significant independent predictors of future drug-treated Diabetes in the Diabetes Risk Score are age, BMI, waist circumference, antihypertensive drug therapy, and history of high blood glucose levels. The Diabetes Risk Score model comprises a concise model that includes only these statistically significant variables and a full model, which includes physical activity and fruit and vegetable consumption.

The San Antonio Heart Study is a long-term, community-based prospective observational study of Diabetes and cardiovascular disease in Mexican Americans and non-Hispanic Caucasians. The study initially enrolled 3,301 Mexican-American and 1,857 non-Hispanic Caucasian men and non-pregnant women in two phases between 1979 and 1988. Participants were 25-64 years of age at enrollment and were randomly selected from low, middle, and high-income neighborhoods in San Antonio, Tex. A 7-8 year follow-up exam followed approximately 73% of the surviving individuals initially enrolled in the study. Baseline characteristics such as medical history of Diabetes, age, sex, ethnicity, BMI, systolic and diastolic blood pressure, fasting and 2-hour plasma glucose levels, fasting serum total cholesterol, LDL, and HDL cholesterol levels, as well as triglyceride levels, were compiled and assessed. A multiple logistic regression model with incident Diabetes as the dependent variable and the aforementioned baseline characteristics were applied as independent variables. Using this model, univariate odds ratios can be computed for each potential risk factor for men and women separately and for both sexes combined. For continuous risk factors, the odds ratios can be presented for a 1-SD increment. A multivariate predicting model with both sexes combined can be developed using a stepwise logistic regression procedure in which the variables that had shown statistically significant odds ratios when examined individually were allowed to enter the model. This multivariable model is then analyzed by ROC curves and 95% CIs of the areas under the ROC curves estimated by non-parametric algorithms such as those described by DeLong (DeLong E. R. et al, (1988) Biometrics 44: 837-45). The results of the San Antonio Heart Study indicate that pre-diabetic subjects have an atherogenic pattern of risk factors (possibly caused by obesity, hyperglycemia, and especially hyperinsulinemia), which may be present for many years and may contribute to the risk of macrovascular disease as much as the duration of clinical Diabetes itself.

Despite the numerous studies and algorithms that have been used to assess the risk of Diabetes or a pre-diabetic condition, the evidence-based, multiple risk factor assessment approach is only moderately accurate for the prediction of short- and long-term risk of manifesting Diabetes, one or more complications related to Diabetes, or a pre-diabetic condition in individual asymptomatic or otherwise healthy subjects. Such risk prediction algorithms can be advantageously used in combination with the T2DBMARKERS of the present invention to distinguish between subjects in a population of interest to determine the risk stratification of developing Diabetes, one or more complications related to Diabetes, or a pre-diabetic condition. The T2DBMARKERS and methods of use disclosed herein provide tools that can be used in combination with such risk prediction algorithms to assess, identify, or diagnose subjects who are asymptomatic and do not exhibit the conventional risk factors.

The data derived from risk prediction algorithms and from the methods of the present invention can be compared by linear regression. Linear regression analysis models the relationship between two variables by fitting a linear equation to observed data. One variable is considered to be an explanatory variable, and the other is considered to be a dependent variable. For example, values obtained from the Archimedes or San Antonio Heart analysis can be used as a dependent variable and analyzed against levels of one or more T2DBMARKERS as the explanatory variables in an effort to more fully define the underlying biology implicit in the calculated algorithm score (see Examples). Alternatively, such risk prediction algorithms, or their individual inputs, which are generally T2DBMARKERS themselves, can be directly incorporated into the practice of the present invention, with the combined algorithm compared against actual observed results in a historical cohort.

A linear regression line has an equation of the form Y=a+bX, where X is the explanatory variable and Y is the dependent variable. The slope of the line is b, and a is the intercept (the value of y when x=0). A numerical measure of association between two variables is the “correlation coefficient,” or R, which is a value between −1 and 1 indicating the strength of the association of the observed data for the two variables. This is also often reported as the square of the correlation coefficient, as the “coefficient of determination” or R²; in this form it is the proportion of the total variation in Y explained by fitting the line. The most common method for fitting a regression line is the method of least-squares. This method calculates the best-fitting line for the observed data by minimizing the sum of the squares of the vertical deviations from each data point to the line (if a point lies on the fitted line exactly, then its vertical deviation is 0). Because the deviations are first squared, then summed, there are no cancellations between positive and negative values.

After a regression line has been computed for a group of data, a point which lies far from the line (and thus has a large residual value) is known as an outlier. Such points may represent erroneous data, or may indicate a poorly fitting regression line. If a point lies far from the other data in the horizontal direction, it is known as an influential observation. The reason for this distinction is that these points have may have a significant impact on the slope of the regression line. Once a regression model has been fit to a group of data, examination of the residuals (the deviations from the fitted line to the observed values) allows one of skill in the art to investigate the validity of the assumption that a linear relationship exists. Plotting the residuals on the y-axis against the explanatory variable on the x-axis reveals any possible non-linear relationship among the variables, or might alert the skilled artisan to investigate “lurking variables.” A “lurking variable” exists when the relationship between two variables is significantly affected by the presence of a third variable which has not been included in the modeling effort.

Linear regression analyses can be used, inter alia, to predict the risk of developing Diabetes, one or more complications related to Diabetes, or a pre-diabetic condition based upon correlating the levels of one or more T2DBMARKERS in a sample from a subject to that subjects' actual observed clinical outcomes, or in combination with, for example, calculated Archimedes risk scores, San Antonio Heart risk scores, or other known methods of diagnosing or predicting the prevalence of Diabetes, one or more complications related to Diabetes, or a pre-diabetic condition. Of particular use, however, are non-linear equations and analyses to determine the relationship between known predictive models of Diabetes and levels of T2DBMARKERS detected in a subject sample. Of particular interest are structural and synactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models. Most commonly used are classification algorithms using logistic regression, which are the basis for the Framingham, Finnish, and San Antonio Heart risk scores. Furthermore, the application of such techniques to panels of multiple T2DBMARKERS is encompassed by or within the ambit of the present invention, as is the use of such combination to create single numerical “risk indices” or “risk scores” encompassing information from multiple T2DBMARKER inputs.

Factor analysis is a mathematical technique by which a large number of correlated variables (such as Diabetes risk factors) can be reduced to fewer “factors” that represent distinct attributes that account for a large proportion of the variance in the original variables (Hanson, R. L. et al, (2002) Diabetes 51: 3120-3127). Thus, factor analysis is well suited for identifying components of Diabetes Mellitus and pre-diabetic conditions such as IGT, IFG, and Metabolic Syndrome. Epidemiological studies of factor “scores” from these analyses can further determine relations between components of the metabolic syndrome and incidence of Diabetes. The premise underlying factor analysis is that correlations observed among a set of variables can be explained by a small number of unique unmeasured variables, or “factors”. Factor analysis involves two procedures: 1) factor extraction to estimate the number of factors, and 2) factor rotation to determine constituents of each factor in terms of the original variables.

Factor extraction can be conducted by the method of principal components. These components are linear combinations of the original variables that are constructed so that each component has a correlation of zero with each of the other components. Each principal component is associated with an “eigen-value,” which represents the variance in the original variables explained by that component (with each original variable standardized to have a variance of 1). The number of principal components that can be constructed is equal to the number of original variables. In factor analysis, the number of factors is customarily determined by retention of only those components that account for more of the total variance than any single original variable (i.e., those components with eigen-values of >1).

Once the number of factors has been established, then factor rotation is conducted to determine the composition of factors that has the most parsimonious interpretation in terms of the original variables. In factor rotation, “factor loadings,” which represent correlations of each factor with the original variables, are changed so that these factor loadings are made as close to 0 or 1 as possible (with the constraint that the total amount of variance explained by the factors remains unchanged). A number of methods for factor rotation have been developed and can be distinguished by whether they require the final set of factors to remain uncorrelated with one another (also known as “orthogonal methods”) or by whether they allow factors to be correlated (“oblique methods”). In interpretation of factor analysis, the pattern of factor loadings is examined to determine which original variables represent primary constituents of each factor. Conventionally, variables that have a factor loading of >0.4 (or less than −0.4) with a particular factor are considered to be its major constituents. Factor analysis can be very useful in constructing T2DBMARKER panels from their constituent components, and in grouping substitutable groups of markers.

Comparison can be performed on test (“subject”) and reference (“control”) samples measured concurrently or at temporally distinct times. An example of the latter is the use of compiled expression information, e.g., a sequence database, which assembles information about expression levels of T2DBMARKERS. If the reference sample, e.g., a control sample is from a subject that does not have Diabetes a similarity in the amount of the T2DBMARKERS in the subject test sample and the control reference sample indicates that the treatment is efficacious. However, a change in the amount of one or more T2DBMARKERS in the test sample and the reference sample can reflect a less favorable clinical outcome or prognosis. “Efficacious” or “effective” means that the treatment leads to an decrease or increase in the amount of one or more T2DBMARKERS, or decrease of serum insulin levels or blood glucose levels in a subject. Assessment of serum insulin or blood glucose levels can be analyzed using standard clinical protocols. Efficacy can be determined in association with any known method for diagnosing or treating Diabetes.

Levels of an effective amount of T2DBMARKER proteins, peptides, nucleic acids, polymorphisms, metabolites, or other analytes also allows for the course of treatment of Diabetes, one or more complications related to Diabetes, or a pre-diabetic condition to be monitored. In this method, a biological sample can be provided from a subject undergoing treatment regimens, e.g., drug treatments, for Diabetes. Such treatment regimens can include, but are not limited to, exercise regimens, dietary supplementation (including without limitation, alpha-lipoic acid, chromium, coenzyme Q10, garlic, magnesium, and omega-3 fatty acids), surgical intervention (such as but not limited to gastric bypass, angioplasty, etc.), and treatment with therapeutics or prophylactics used in subjects diagnosed or identified with Diabetes, one or more complications related to Diabetes, or a pre-diabetic condition, such as for example, diabetes-modulating agents as defined herein. If desired, biological samples are obtained from the subject at various time points before, during, or after treatment. Levels of an effective amount of T2DBMARKER proteins, peptides, nucleic acids, polymorphisms, metabolites, or other analytes can then be determined and compared to a reference value, e.g. a control subject or population whose diabetic state is known or an index value or baseline value. The reference sample or index value or baseline value may be taken or derived from one or more subjects who have been exposed to the treatment, or may be taken or derived from one or more subjects who are at low risk of developing Diabetes, one or more complications related to Diabetes, or a pre-diabetic condition, or may be taken or derived from subjects who have shown improvements in Diabetes risk factors as a result of exposure to treatment. Alternatively, the reference sample or index value or baseline value may be taken or derived from one or more subjects who have not been exposed to the treatment. For example, samples may be collected from subjects who have received initial treatment for Diabetes, one or more complications related to Diabetes, or a pre-diabetic condition and subsequent treatment for Diabetes, one or more complications related to Diabetes, or a pre-diabetic condition to monitor the progress of the treatment. A reference value can also comprise a value derived from risk prediction algorithms or computed indices from population studies such as those disclosed herein.

The T2DBMARKERS of the present invention can thus be used to generate a “reference expression profile” which comprises a pattern of expression levels of T2DBMARKERS detected in those subjects who do not have Diabetes, one or more complications related to Diabetes, or a pre-diabetic condition such as impaired glucose tolerance, and would not be expected to develop Diabetes, one or more complications related to Diabetes, or a pre-diabetic condition. The T2DBMARKERS disclosed herein can also be used to generate a “subject expression profile” comprising a pattern of expression levels of T2DBMARKERS taken from subjects who have Diabetes, one or more complications related to Diabetes, or a pre-diabetic condition like impaired glucose tolerance. The subject expression profiles can be compared to a reference expression profile to diagnose or identify subjects at risk for developing Diabetes, one or more complications related to Diabetes, or a pre-diabetic condition, to monitor the progression of disease, as well as the rate of progression of disease, including development or risk of development of complications related to type 2 Diabetes or pre-diabetic conditions, and to monitor the effectiveness of Diabetes or pre-diabetic condition treatment modalities. The reference and subject expression profiles of the present invention can be contained in a machine-readable medium, such as but not limited to, analog tapes or digital media like those readable by a VCR, CD-ROM, DVD-ROM, USB flash media, among others. Such machine-readable media can also contain additional test results, such as, without limitation, measurements of conventional Diabetes risk factors like systolic and diastolic blood pressure, blood glucose levels, insulin levels, BMI indices, and cholesterol (LDL and HDL) levels. Alternatively or additionally, the machine-readable media can also comprise subject information such as medical history and any relevant family history. The machine-readable media can also contain information relating to other Diabetes-risk algorithms and computed indices such as those described herein.

Differences in the genetic makeup of subjects can result in differences in their relative abilities to metabolize various agents, which may modulate the symptoms or risk factors of Diabetes, one or more complications related to Diabetes, or a pre-diabetic condition. Subjects that have Diabetes, one or more complications related to Diabetes, or a pre-diabetic condition, or at risk for developing Diabetes, one or more complications related to Diabetes, or a pre-diabetic condition can vary in age, ethnicity, body mass index (BMI), total cholesterol levels, blood glucose levels, blood pressure, LDL and HDL levels, and other parameters. Accordingly, use of the T2DBMARKERS disclosed herein allow for a pre-determined level of predictability that a putative therapeutic or prophylactic to be tested in a selected subject will be suitable for treating or preventing Diabetes, a pre-diabetic condition, or complications thereof in the subject.

To identify therapeutics or agents that are appropriate for a specific subject, a test sample from the subject can be exposed to a therapeutic agent or a drug, and the level of one or more of T2DBMARKER proteins, nucleic acids, polymorphisms, metabolites or other analytes can be determined. The level of one or more T2DBMARKERS can be compared to a sample derived from the subject at a first period of time before and at a second period of time after treatment or exposure to a therapeutic agent or a drug, or can be compared to samples derived from one or more subjects who have shown improvements in Diabetes, one or more complications related to Diabetes, or pre-diabetic condition risk factors as a result of such treatment or exposure. Examples of such therapeutics or agents frequently used in Diabetes treatments, and may modulate the symptoms or risk factors of Diabetes include, but are not limited to, sulfonylureas like glimepiride, glyburide (also known in the art as glibenclamide), glipizide, gliclazide; biguanides such as metformin; insulin (including inhaled formulations such as Exubera), and insulin analogs such as insulin lispro (Humalog), insulin glargine (Lantus), insulin detemir, and insulin glulisine; peroxisome proliferator-activated receptor-γ (PPAR-γ) agonists such as the thiazolidinediones including troglitazone (Rezulin), pioglitazone (Actos), rosiglitazone (Avandia), and isaglitzone (also known as netoglitazone); dual-acting PPAR agonists such as BMS-298585 and tesaglitazar; insulin secretagogues including metglitinides such as repaglinide and nateglinide; analogs of glucagon-like peptide-1 (GLP-1) such as exenatide (AC-2993) and liraglutide (insulinotropin); inhibitors of dipeptidyl peptidase IV like LAF-237; pancreatic lipase inhibitors such as orlistat; α-glucosidase inhibitors such as acarbose, miglitol, and voglibose; and combinations thereof, particularly metformin and glyburide (Glucovance), metformin and rosiglitazone (Avandamet), and metformin and glipizide (Metaglip). Such therapeutics or agents have been prescribed for subjects diagnosed with Diabetes, one or more complications related to Diabetes, or a pre-diabetic condition, and may modulate the symptoms or risk factors of Diabetes, one or more complications related to Diabetes, or a pre-diabetic condition (herein, “diabetes-modulating agents”).

A subject sample can be incubated in the presence of a candidate agent and the pattern of T2DBMARKER expression in the test sample is measured and compared to a reference profile, e.g., a Diabetes reference expression profile or a non-Diabetes reference expression profile or an index value or baseline value. The test agent can be any compound or composition or combination thereof. For example, the test agents are agents frequently used in Diabetes treatment regimens and are described herein.

Table 1 comprises the five hundred and forty-eight (548) T2DBMARKERS of the present invention. One skilled in the art will recognize that the T2DBMARKERS presented herein encompasses all forms and variants, including but not limited to, polymorphisms, isoforms, mutants, derivatives, precursors including nucleic acids, receptors (including soluble and transmembrane receptors), ligands, and post-translationally modified variants, as well as any multi-unit nucleic acid, protein, and glycoprotein structures comprised of any of the T2DBMARKERS as constituent subunits of the fully assembled structure.

TABLE 1
T2DBMARKERS

| T2DBMARKER | Common Name | Alternative Name |
|---|---|---|
| 1 | Serpina 3M | C-terminal fragment of a predicted protein, similar to |
| | | serine protease inhibitor 2.4 |
| 2 | Spin 2a | |
| 3 | Fetuin beta | Fetub; Fetuin β; Fetuin B |
| 4 | Apolipoprotein C-III | Apoc3 |
| | precursor | |
| 5 | Predicted protein, similar to | Apoc2, predicted |
| | Apolipoprotein C2 | |
| 6 | Alpha-2-HS-glycoprotein | α-2-HS-glycoprotein; Ahsg; Fetuin α; Fetuin A; |
| | | Aa2-066 |
| 7 | T-kininogen II precursor | |
| 8 | Alpha-1-macroglobulin | α-1-macroglobulin; A2MG; Pzp; pregnancy-zone |
| | | protein |
| 9 | Serpin C1 | Serine/cysteine proteinase inhibitor, clade C, |
| | | member 1 (predicted) |
| 10 | Coagulation factor 2 | F2 |
| 11 | Inter-alpha-inhibitor H4 | ITIH4 |
| | heavy chain | |
| 12 | Vitamin D binding protein | Gc; VTDB |
| | prepeptide | |
| 13 | Low-molecular weight T- | Kininogen; LMW T-kininogen I precursor; major |
| | kininogen I precursor | acute phase alpha-1 protein precursor |
| 14 | Apolipoprotein A-1 | Preapolipoprotein A-1; ApoA1 |
| 15 | Predicted protein, similar to | Apoc2, precursor |
| | apolipoprotein C-II | |
| | precursor | |
| 16 | Thrombin | Prothrombin precursor; THRB |
| 17 | Apolipoprotein E | ApoE |
| 18 | Liver regeneration-related | Tf |
| | protein LRRG03 | |
| 19 | Apolipoprotein A-IV | ApoA4 |
| 20 | Alpha-1-inhibitor 3 | LOC297568 |
| | precursor | |
| 21 | XP_579384 | |
| 22 | Histidine-rich glycoprotein | Hrg |
| 23 | XP_579477 | |
| 24 | Complement component C9 | C9 |
| | precursor | |
| 25 | Apolipoprotein H | ApoH |
| 26 | B-factor, properdin | Cfb |
| 27 | Hemopexin | Hpx |
| 28 | Calnexin | Ca(2+)-binding phosphoprotein p90 |
| 29 | Reg3a | Rn.11222; regenerating islet-derived 3 alpha |
| 30 | LOC680945 | Rn.1414; similar to stromal cell-derived factor 2-like 1 |
| 31 | Pap | Rn.9727; pancreatitis-associated protein |
| 32 | Ptf1a | Rn.10536; Pancreas specific transcription factor, 1a |
| 33 | Mat1a | Rn.10418; methionine adenosyltransferase I, alpha |
| 34 | Nupr1 | Rn.11182; nuclear protein 1 |
| 35 | Rn.128013 | |
| 36 | Chac1 (predicted) | Rn.23367; ChaC; cation transport regulator-like 1 |
| 37 | Slc7a3 | Rn.9804; solute carrier family 7 (cationic amino acid |
| | | transporter, y+ system), member 3 |
| 38 | LOC312273 | Rn.13006; trypsin V-A |
| 39 | Rn.47821 | |
| 40 | Ptger3 | Rn.10361; prostaglandin E receptor 3 (subtype EP3 |
| 41 | RGD1562451 | Rn.199400; similar to Pabpc4 predicted protein |
| 42 | RGD1566242 | Rn.24858; similar to RIKEN cDNA 1500009M05 |
| 43 | Cyp2d26 | Rn.91355; Cytochrome P450, family 2, subfamily d, |
| | | polypeptide 26 |
| 44 | Rn.17900 | Similar to aldehyde dehydrogenase 1 family, |
| | | member L2 |
| 45 | LOC286960 | Rn.10387; preprotrypsinogen IV |
| 46 | Gls2 | Rn.10202; glutaminase 2 (liver, mitochondrial) |
| 47 | Nme2 | Rn.927; expressed in non-metastatic cells 2 |
| 48 | Rn.165714 | |
| 49 | P2rx1 | Rn.91176; purinergic receptor PX2, ligand-gated ion |
| | | channel, 1 |
| 50 | Pdk4 | Rn.30070; pyruvate dehydrogenase kinase, |
| | | isoenzyme 4 |
| 51 | Amy1 | Rn.116361; amylase 1, salivary |
| 52 | Cbs | Rn.87853; cystathionine beta synthase |
| 53 | Mte1 | Rn.37524; mitochondrial acyl-CoA thioesterase 1 |
| 54 | Spink1 | Rn.9767; serine protease inhibitor, Kazal type 1 |
| 55 | Gatm | Rn.17661; glycine amidinetransferase (L- |
| | | arginine:glycine amidinotransferase) |
| 56 | Tmed6_predicted | Rn.19837; transmembrane emp24 protein transport |
| | | domain containing 6 |
| 57 | Tff2 | Rn.34367; trefoil factor 2 (spasmolytic protein 1) |
| 58 | Hsd17b13 | Rn.25104; hydroxysteroid (17-beta) dehydrogenase |
| | | 13 |
| 59 | Rn.11766 | Similar to LRRGT00012 |
| 60 | Gnmt | Rn.11142; glycine N-methyltransferase |
| 61 | Pah | Rn.1652; phenylalanine hydroxylase |
| 62 | Serpini2 | Rn.54500; serine/cysteine proteinase inhibitor, clade |
| | | I, member 2 |
| 63 | RGD1309615 | Rn.167687 |
| 64 | LOC691307 | Rn.79735; similar to leucine rich repeat containing |
| | | 39 isoform 2 |
| 65 | Eprs | Rn.21240; glutamyl-prolyl-tRNA synthetase |
| 66 | Pck2_predicted | Rn.35508; phosphoenolpyruvate carboxykinase 2 |
| | | (mitochondrial) |
| 67 | Chd2_predicted | Rn.162437; chromodomain helicase DNA binding |
| | | protein 2 |
| 68 | Rn.53085 | |
| 69 | Rn.12530 | |
| 70 | NIPK | Rn.22325; tribbles homolog; cDNA clone RPCAG66 |
| | | 3′ end, mRNA sequence |
| 71 | Slc30a2 | Rn.11135; solute carrier family 30 (zinc transporter), |
| | | member 2 |
| 72 | Serpina10 | Rn.10502; serine/cysteine peptidase inhibitor, clade |
| | | A, member 10 |
| 73 | Cfi | Rn.7424; complement factor I |
| 74 | Cckar | Rn.10184; cholecystokinin A receptor |
| 75 | LOC689755 | Rn.151728; LOC689755 |
| 76 | Bhlhb8 | Rn.9897; basic helix-loop-helix domain containing |
| | | class B, 8 |
| 77 | Anpep | Rn.11132; alanyl (membrane) aminopeptidase) |
| 78 | Asns | Rn.11172; asparagine synthetase |
| 79 | Slc7a5 | Rn.32261; solute carrier family 7 (cationic amino |
| | | acid transporter, y+ system), member 5 |
| 80 | Usp43_predicted | Rn.12678; ubiquitin specific protease 43 |
| 81 | Csnk1a1 | Rn.23810; casein kinase 1, alpha 1 |
| 82 | Cml2 | Rn.160578; camello-like 2 |
| 83 | Pabpc4 | Rn.199602 |
| 84 | Gjb2 | Rn.198991; gap junction membrane channel protein |
| | | beta 2 |
| 85 | Ngfg | Rn.11331; nerve growth factor, gamma |
| 86 | Clca2_predicted | Rn.48629 |
| 87 | RGD1565381 | Rn.16083; similar to RIKEN cDNA 181003M07 |
| 88 | Qscn6 | Rn.44920; quiescin Q6 |
| 89 | Cldn10_predicted | Rn.99994; claudin 10 |
| 90 | Spink3 | Rn.144683; serine protease inhibitor, Kazal type 3 |
| 91 | LOC498174 | Rn.163210; similar to NipSnap2 protein |
| | | (glioblastoma amplified sequence) |
| 92 | Rn.140163 | Similar to methionine-tRNA synthetase |
| 93 | Cyr61 | Rn.22129; cysteine rich protein 61 |
| 94 | RGD1307736 | Rn.162140; Similar to KIAA0152 |
| 95 | Ddit3 | Rn.11183; DNA damage inducible transcript 3 |
| 96 | Reg1 | Rn.11332; regenerating islet derived 1 |
| 97 | Eif4b | Rn.95954; eukaryotic translation initiation factor 4B |
| 98 | Rnase4 | Rn.1742; ribonuclease, RNase A family 4 |
| 99 | Cebpg | Rn.10332; CCAAT/enhancer binding protein |
| | | (C/EBP), gamma |
| 100 | siat7D | Rn.195322; alpha-2,6-sialyltransferase ST6GalNAc |
| | | IV |
| 101 | Herpud1 | Rn.4028; homocysteine-inducible, ubiquitin-like |
| | | domain member 1 |
| 102 | Unknown rat cDNA | |
| 103 | Gcat | Rn.43940; glycine C-acetyltransferase (2-amino-3- |
| | | ketobutyrate-coenzyme A ligase) |
| 104 | RGD1562860 | Rn.75246; similar to RIKEN cDNA 2310045A20 |
| 105 | pre-mtHSP70 | Rn.7535; 70 kD heat shock protein precursor; |
| | | Hspa9a_predicted; heat shock 70 kD protein 9A |
| 106 | Dbt | Rn.198610; dihydrolipoamide branched chain |
| | | transacylase E2 |
| 107 | Bspry | Rn.53996; B-box and SPRY domain containing |
| 108 | Fut1 | Rn.11382; fucosyltransferase 1 |
| 109 | Rpl3 | Rn.107726; ribosomal protein L3 |
| 110 | Rn.22481 | Similar to NP_083520.1 acylphosphatase 2, muscle |
| | | type |
| 111 | Vldlr | Rn.9975; very low density lipoprotein receptor |
| 112 | RGD1311937 | Rn.33652; similar to MGC17299 |
| 113 | RGD1563144 | Rn.14702; Similar to EMeg32 protein |
| 114 | Rn.43268 | |
| 115 | Ddah1 | Rn.7398; dimethylarginine dimethylaminohydrolase 1 |
| 116 | RAMP4 | Rn.2119; ribosome associated membrane protein 4 |
| 117 | Rn.169405 | |
| 118 | Ccbe1_predicted | Rn.199045; collagen and calcium binding EGF |
| | | domains 1 |
| 119 | Dnajc3 | Rn.162234; DnaJ (Hsp40) homolog, subfamily C, |
| | | member 3 |
| 120 | Mtac2d1 | Rn.43919; membrane targeting (tandem)C2 domain |
| | | containing 1 |
| 121 | RGD1563461 | Rn.199308 |
| 122 | Gimap4 | Rn.198155; GTPase, IMAP family member 4 |
| 123 | Klf2_predicted | Rn.92653; Kruppel-like factor 2 (lung) |
| 124 | RGD1309561 | Rn.102005; similar to FLH31951 |
| 125 | NAP22 | Rn.163581 |
| 126 | Sfrs3_predicted | Rn.9002; splicing factor, arginine/serine-rich 3 |
| | | (SRp30) |
| 127 | Rn.6731 | |
| 128 | Cd53 | Rn.31988; CD53 antigen |
| 129 | RGD1561419 | Rn.131539; similar to RIKEN cDNA 6030405P05 |
| | | gene; ARHGAP30; Hs.389374; Rho GTPase |
| | | activating protein |
| 130 | Il2rg | Rn.14508; interleukin 2 receptor, gamma |
| 131 | LOC361346 | Rn.31250; similar to chromosome 18 open reading |
| | | frame 54 |
| 132 | Plac8_predicted | Rn.2649; placenta-specific 8 |
| 133 | LOC498335 | Rn.6917; similar to small inducible cytokine B13 |
| | | precursor (CXCL13) |
| 134 | Igfbp3 | Rn.26369; insulin-like growth factor binding protein 3 |
| 135 | Ptprc | Rn.90166; Hs.192039; protein tyrosine phosphatase, |
| | | receptor type C; CD45 |
| 136 | RT1-Aw2 | Rn.40130; RT1 class Ib, locus Aw2 |
| 137 | Rac2 | Rn.2863; RAS-related C3 botulinum substrate 2 |
| 138 | Rn.9461 | |
| 139 | Fos | Rn.103750; FBJ murine osteosarcoma viral |
| | | oncogene homolog |
| 140 | Sgne1 | Rn.6173; secretory granule neuroendocrine protein 1 |
| 141 | Fcgr2b | Rn.33323; Fc receptor, IgG, low affinity IIb |
| 142 | Slfn8 | Rn.137139; Schlafen 8 |
| 143 | Rab8b | Rn.10995; RAB8B, member RAS oncogene family |
| 144 | Rn.4287 | |
| 145 | RGD1306939 | Rn.95357; similar to mKIAA0386 protein |
| 146 | Tnfrsf26_predicted | Rn.162508; tumor necrosis factor receptor |
| | | superfamily, member 26 |
| 147 | Ythdf2_predicted | Rn.21737; YTH domain family 2 |
| 148 | RGD1359202 | Rn.10956; similar to immunoglobulin heavy chain 6 |
| | | (Igh-6); IGHG1 in humans; immunoglobulin heavy |
| | | constant gamma 1 |
| 149 | RGD1562855 | Rn.117926; similar to Ig kappa chain |
| 150 | Igha_mapped | Rn.109625; immunoglobulin heavy chain (alpha |
| | | polypeptide) (mapped) |
| 151 | Ccl21b | Rn.39658; chemokine (C-C motif) ligand 21b |
| | | (serine) |
| 152 | IGHM | Rn.201760; Hs.510635; IGHM; immunoglobulin |
| | | heavy constant mu |
| 153 | LCK | Rn.22791; Hs.470627; lymphocyte protein tyrosine |
| | | kinase |
| 154 | ARHGD1B | Rn.15842; Hs.507877; Rho GDP dissociation |
| | | inhibitor (CDI) beta |
| 155 | CD38 | Rn.11414; Hs.479214; CD38 antigen |
| 156 | S100B | Rn.8937; Hs.422181; S100 calcium binding protein |
| | | B, beta polypeptide |
| 157 | RGD1306952 | Rn.64439; Similar to Ab2-225 |
| 158 | Dmrt2 | Rn.11448; Doublesex and mab-3 related |
| | | transcription factor 2 (predicted) |
| 159 | AA819893 | Rn.148042; unknown cDNA |
| 160 | Gpr176 | Rn.44656; G-protein coupled receptor 176 |
| 161 | Tmem45b | Rn.42073; transmembrane protein 45b |
| 162 | Nfkbil1 | Rn.38632; nuclear factor of kappa light polypeptide |
| | | gene enhancer in B-cells inhibitor-like 1 |
| 163 | Dctn2 | Rn.101923; Dynactin 2 |
| 164 | Itpkc | Rn.85907; Inositol 1,4,5-trisphosphate 3-kinase C |
| 165 | BM389613 | Rn.171826; unknown cDNA |
| 166 | Prodh2 | Rn.4247; proline dehydrogenase (oxidase) 2 |
| 167 | BF288777 | Rn.28947; unknown cDNA |
| 168 | Abi3 | Rn.95169; ABI gene family, member 3 |
| 169 | AW531966 | Rn.8606; unknown cDNA |
| 170 | RGD1560732 | Rn.100399; Similar to LIM and senescent cell |
| | | antigen-like domains 1 (predicted) |
| 171 | Oxsr1 | Rn.21097; oxidative-stress responsive 1 (predicted) |
| 172 | MGC114531 | Rn.39247; unknown cDNA |
| 173 | BF418465 | Rn.123735; unknown cDNA |
| 174 | LOC690911 | Rn.25022; similar to Msx2-interacting protein |
| | | (SPEN homolog) |
| 175 | Pex6 | Rn.10675; Peroxisomal biogenesis factor 6 |
| 176 | RGD1311424 | Rn.57800; similar to hypothetical protein FLJ38348 |
| | | (predicted) |
| 177 | AI013238 | Rn.135595; unknown cDNA |
| 178 | BI288719 | Rn.45106; unknown cDNA |
| 179 | Evp1 | Rn.19832; envoplakin (predicted) |
| 180 | SERPINE2 | Rn.2271; Hs.38449; serine (or cysteine) proteinase |
| | | inhibitor clade E member 2 |
| 181 | C20orf160 | Rn.6807; Hs.382157; C20orf160 predicted; cystein |
| | | type endopeptidase |
| 182 | AI072137 | Rn.33396; Transcribed locus |
| 183 | LOC338328 | Rn.7294; Hs.426410; high density lipoprotein |
| | | binding protein; RGD1564237_predicted |
| 184 | PTPRR | Rn.6277; Hs.506076; protein tyrosine phosphatase |
| | | receptor type R |
| 185 | LYPLA3 | Rn.93631; Hs.632199; Lysophospholipase 3 |
| 186 | CYYR1 | Rn.1528; Hs.37445; cysteine-tyrosine-rich 1 |
| | | membrane associated protein |
| 187 | SOX17 | Rn.7884; Hs.98367; SRY-box gene 17 |
| 188 | LY6H | Rn.40119 |
| 189 | SEMA3G | Rn.32183; HS.59729; Semaphorin 3G |
| 190 | C1QTNF1 | Rn.53880; Hs.201398; C1q and tumor necrosis |
| | | factor related protein 1 |
| 191 | ADCY4 | Rn.1904; Hs.443428; adenylate cyclase 4 |
| 192 | RBP7 | Rn.13092; Hs.422688; retinol binding protein 7; |
| | | RGD1562168_predicted |
| 193 | ADRB3 | Rn.10100; Hs.2549; adrenergic receptor beta-3 |
| 194 | NR1H3 | Rn.11209; Hs.438863; nuclear receptor subfamily, |
| | | group H, member 3 |
| 195 | TMEFF1 | Rn.162809; Hs.657066; transmembrane protein with |
| | | EGF-like and two follistatin-like domains 1 |
| 196 | TIMP-4 | Rn.155651; Hs.591665; Tissue inhibitor of |
| | | metalloproteinase 4 |
| 197 | CYP4F8 (human) | Rn.10170; Hs.268554; cytochrome P450, family 4, |
| | | subfamily F, polypeptide 8 |
| 198 | FOLR1 | Rn.6912; Hs.73769; folate receptor 1 |
| 199 | SCD2 | Rn.83595; Hs.558396; stearoyl-CoA desaturase 2 |
| 200 | KIAA2022 | Rn.62924; Hs.124128; DNA polymerase activity |
| 201 | GK | Rn.44654; Hs.1466; glycerol kinase; Gyk |
| 202 | OCLN | Rn.31429; Hs.592605; occluding |
| 203 | SPINT2 | Rn.3857; Hs.31439; serine peptidase inhibitor, |
| | | Kunitz type, 2 |
| 204 | RBM24 | Rn.164640; Hs.519904; RNA binding motif protein |
| | | 24 |
| 205 | SLC25A13 | Rn.14686; Hs.489190; solute carrier family 25, |
| | | member 13 (citrin) |
| 206 | TPMT | Rn.112598; Hs.444319; thiopurine S- |
| | | methyltransferase |
| 207 | KRT18 | Rn.103924; Hs.406013; keratin 18; keratin complex |
| | | 1, acidic, gene 18; Krt1-18 |
| 208 | Unknown | Rn.153497 |
| 209 | C2orf40 | Rn.16593; Hs.43125; chromosome 2 open reading |
| | | frame 40 |
| 210 | LOC440335 | Rn.137175; Hs.390599; hypothetical gene supported |
| | | by BC022385; RGD1563547; RGE1563547 |
| | | (predicted) |
| 211 | BEXL1 | Rn.9287; Hs.184736; brain expressed X-linked-like |
| | | 1; BI289546; brain expressed X-linked 4 |
| 212 | CYB561 | Rn.14673; Hs.355264; cytochrome b-561 |
| 213 | AMOT | Rn.149241; Hs.528051; angiomotin |
| 214 | SQLE | Rn.33239; Hs.71465; squalene epoxidase |
| 215 | ANKRD6 | Rn.45844; Hs.656539; ankyrin repeat domain 6 |
| 216 | CCDC8 | Rn.171055; Hs.97876; coiled-coil domain containing 8 |
| 217 | KRT8 | Rn.11083; Hs.533782; keratin 8 |
| 218 | WWC1 (Mus musculus) | Rn.101912; Hs.484047; WW and C2 domain |
| | | containing 1; RGD1308329; similar to KIAA0869 |
| | | protein (predicted) |
| 219 | PFKP | Rn.2278; Hs.26010; phosphofructokinase |
| 220 | PEBP1 | Rn.29745; Hs.433863; phosphatidylethanolamine |
| | | binding protein 1 |
| 221 | SLC7A1 | Rn.9439; Hs.14846; solute carrier family 7 (cationic |
| | | amino acid transport, y+ system), member 1 |
| 222 | GSTM1 | Rn.625; Hs.301961; glutathione S-transferase M1; |
| | | glutathione metabolism, mu 1 |
| 223 | CCL5 | Rn.8019; Hs.514821; chemokine (C-C motif) ligand 5 |
| 224 | STEAP1 | Rn.51773; Hs.61635; six transmembrane epithelial |
| | | antigen of the prostate 1 |
| 225 | IAH1 | Rn.8209; HS.656852; isoamyl acetate-hydrolyzing |
| | | esterase 1 homolog (S. cerevisiae) |
| 226 | GNA14 | Rn.35127; Hs.657795; guanine nucleotide binding |
| | | protein (G protein), alpha 14 |
| 227 | TMEM64 | Rn.164935; Hs.567759; transmembrane protein 64 |
| 228 | CCL11 | Rn.10632; Hs.54460; chemokine (C-C motif) ligand |
| | | 11 |
| 229 | CNN1 | Rn.31788; Hs.465929; Calponin 1 |
| 230 | GGH | Rn.10260; Hs.78619; gamma-glutamyl hydrolase |
| 231 | TPM3 | Rn.17580; Hs.645521; tropomyosin 3 |
| 232 | PCDH7 | Rn.25383; Hs.570785; protocadherin 7 |
| 233 | FHL2 | Rn.3849; Hs.443687; Four and a half LIM domains 2 |
| 234 | COL11A1 | Rn.260; Hs.523446; Collagen, type XI, alpha 1 |
| 235 | EMB | Rn.16221; Hs.645309; Embigin homolog (mouse) |
| 236 | ISG15 | Rn.198318; Hs.458485; ISG15 ubiquitin-like |
| | | modifier |
| 237 | CRYAB | Rn.98208; Hs.408767; crystalline, alpha B |
| 238 | ACADSB | Rn.44423; Hs.81934; Acyl-Coenzyme A |
| | | dehydrogenase |
| 239 | Unknown | Rn.7699; Rn.7699; IMAGE clone BC086433 |
| 240 | ABCA1 | Rn.3724; Hs.429294; ATP-binding cassette, |
| | | subfamily A (ABC1), member 1 |
| 241 | ACSM3 | Rn.88644; Hs.653192; Acyl-CoA synthetase |
| | | medium-chain family member 3 |
| 242 | ACTA2 | Rn.195319; Hs.500483; Actin, alpha 2, smooth |
| | | muscle, aorta |
| 243 | RAMP3 | Rn.48672; Hs.25691; receptor (G-protein coupled; |
| | | calcitonin) activity modifying protein 3 |
| 244 | DDEF1 | Rn.63466; Hs.655552; development and |
| | | differentiation enhancing factor 1 |
| 245 | NIPSNAP3A | Rn.8287; Hs.591897; Nipsnap homolog 3A (C. elegans) |
| 246 | Unknown | Rn.9546 |
| 247 | GPR64 | Rn.57243; Hs.146978; G protein-coupled receptor 64 |
| 248 | SGCB | Rn.98258; Hs.428953; sarcoglycan, beta; AI413058; |
| | | 43 kDa dystrophin-associated glycoprotein (43DAG) |
| 249 | BM389408 | Rn.146540; Transcribed locus |
| 250 | RGD1310037_predicted | Rn.199679; Transcribed locus |
| 251 | CALML3 | Rn.105124; Hs.239600; calmodulin-like 3 |
| 252 | LOC645638 | Rn.41321; Hs.463652; similar to WDNM1-like |
| | | protein |
| 253 | Upk3b_predicted | Rn.6638; transcribed locus |
| 254 | SCEL | Rn.34468; Hs.534699; sciellin |
| 255 | BNC1 | Rn.26595; Hs.459153; Basonuclin 1; BF411725 |
| 256 | FGL2 | Rn.64635; Hs.520989; fibrinogen-like 2 |
| 257 | UPK1B | Rn.9134; Hs.271580; uroplakin 1B |
| 258 | CTDSPL | Rn.37030; Hs.475963; CTD (carboxy-terminal |
| | | domain, RNA polymerase II, polypeptide A) small |
| | | phosphatase-like |
| 259 | PIK3R1 | Rn.163585; Hs.132225; pbosphoinositide-3-kinase, |
| | | regulatory subunit (p85 alpha) |
| 260 | POLA2 | Rn.153998; Hs.201897; polymerase (DNA directed), |
| | | alpha 2 (70 kD subunit); AI175779 |
| 261 | SPTBN1 | Rn.93208; Hs.659362; spectrin, beta, non- |
| | | erythrocytic 1 |
| 262 | RTEL1 | Rn.98315; Hs.434878; regulator of telomere |
| | | elongation helicase 1 |
| 263 | MSLN | Rn.18607; Hs.08488; mesothelin |
| 264 | ARVCF | Rn.220; Hs.655877; armadillo repeat gene deleted in |
| | | velocardiofacial syndrome; Comt; catechol-O- |
| | | methyltransferase |
| 265 | ALB | Rn.9174; Hs.418167; albumin |
| 266 | SLC6A4 | Rn.1663; Hs.591192; solute carrier family 6 |
| | | (neurotransmitter transporter, serotonin), member 4 |
| 267 | Unknown | Rn.26537 |
| 268 | BI302615 | Rn.44072; Transcribed locus |
| 269 | Unknown | Rn.199355 |
| 270 | MRPL4 | Rn.13113 |
| 271 | GPR109A | Rn.79620; Hs.524812; G protein-coupled receptor |
| | | 109A; BI296811 |
| 272 | THBS1 | Rn.185771; Hs.164226; thrombospondin 1 |
| 273 | ANGPTL4 | Rn.119611; Hs.9613; angiopoietin-like 4 |
| 274 | THBS2 | Rn.165619; Hs.371147; thrombospondin 2 |
| 275 | PCK1 | Rn.104376; Hs.1872; phosphoenolpyruvate |
| | | carboxykinase 1 |
| 276 | UCP3 | Rn.9902; Hs.101337; uncoupling protein 3 |
| 277 | CYFIP2 | Rn.44008; Hs.519702; cytoplasmic FMR1 |
| | | interacting protein 2 |
| 278 | LOC646851 | Rn.199989; hypothetical protein |
| 279 | DSP | Rn.54711; Hs.519873; desmoplakin |
| 280 | RNF128 | Rn.7002; Hs.496542; ring finger protein 128 |
| 281 | WDR78 | Rn.22852; Hs.49421; WD repeat domain 78 |
| 282 | SLC16A12 | Rn.166976; Hs.530338; solute carrier family 16, |
| | | member 12 |
| 283 | GRAMD1B | Rn.18035; Hs.144725; GRAM domain containing |
| | | 1B |
| 284 | HPN | Rn.11139; Hs.182385; hepsin (transmembrane |
| | | protease, serine 1) |
| 285 | RRAGD | Rn.66516; Hs.485938; Ras-related GTP binding D |
| 286 | MDF1 | Rn.43395; Hs.520119; MyoD family inhibitor |
| 287 | LTB4DH | Rn.10656; Hs.584864; leukotriene B4 12- |
| | | hydroxydehydrogenase |
| 288 | CELSR2 | Rn.2912; Hs.57652; cadherin, EGF LAG seven-pass |
| | | G-type receptor 2 |
| 289 | LRP4 | Rn.21381; Hs.4930; low density lipoprotein |
| | | receptor-related protein 4 |
| 290 | TPCN2 | Rn.138237; Hs.131851; two pore calcium channel |
| | | protein 2 |
| 291 | TMOD1 | Rn.1646; Hs.494595; tropomodulin 1 |
| 292 | USP2 | Rn.92548; Hs.524085; ubiquitin specific peptidase 2 |
| 293 | SLC16A6 | Rn.54795; Hs.42645; solute carrier family 16, |
| | | member 6 |
| 294 | ATP1A1 | Rn.2992; Hs.371889; ATPase, Na+/K+ transporting, |
| | | alpha 1 polypeptide |
| 295 | CSRP2 | Rn.94754; Hs.530904; cysteine and glycine-rich |
| | | protein 2 |
| 296 | Unknown | Rn.144632 |
| 297 | SLC19A2 | Rn.19386; Hs.30246; solute carrier family 19 |
| | | (thiamine transporter), member 2 |
| 298 | HRSP12 | Rn.6987; Hs.18426; heat-responsive protein 12 |
| 299 | Fkbp11 | Rn.100569; RK506 binding protein 11 |
| 300 | Ace | Rn.10149; angiotensin I converting enzyme |
| | | (peptidyl-dipeptidase A) I |
| 301 | Cyp4f4 (rat) | Rn.10170; cytochrome P450, family 5, subfamily 4, |
| | | polypeptide 4 |
| 302 | BI274837 | Rn.101798; transcribed locus |
| 303 | Hyou1 | Rn.10542; hypoxia up-regulated 1 |
| 304 | MI15 | Rn.106040; myeloid/lymphoid or mixed-lineage |
| | | leukemia 5 (trithorax homolog, Drosophila) |
| 305 | Tcf7 | Rn.106335; transcription factor 7, T-cell specific |
| | | (predicted) |
| 306 | Arf3 | Rn.106440; ADP-ribosylation factor 3 |
| 307 | Mia1 | Rn.10660; melanoma inhibitory activity 1 |
| 308 | Sat | Rn.107986; spermidine/spermine N1-acetyl |
| | | transferase (mapped) |
| 309 | Mpg | Rn.11241; N-methylpurine-DNA glycosylase |
| 310 | BE115368 | Rn.118708; transcribed locus |
| 311 | BI281874 | Rn.125724; Kelch-like 23 (Drosophila)(predicted) |
| 312 | Lcp1 | Rn.14256; lymphocyte cytosolic protein 1 |
| 313 | RGD1306682 | Rn.143893; similar to RIKEN cDNA 1810046J19 |
| | | (predicted) |
| 314 | AI502114 | RN.148916; ATP-binding cassette, sub-family A |
| | | (ABC1), member 1 |
| 315 | AA899202 | Rn.14907; transcribed locus |
| 316 | BI275261 | Rn.157564; transcribed locus |
| 317 | AW532939 | Rn.158403; transcribed locus |
| 318 | Isg20 | Rn.16103; interferon stimulated exonuclease 20 |
| 319 | AI137294 | Rn.161824; similar to Mkrn1protein |
| 320 | BE107848 | Rn.162933; similar to FYVE, RhoGEF and PH |
| | | domain containing 6 (predicted) |
| 321 | BM390584 | Rn.163173; cDNA clone IMAGE: 7455180, |
| | | containing frame-shift errors |
| 322 | Slc25a15 | Rn.163331; solute carrier family 25 (mitochondrial |
| | | carrier; ornithine transporter) member 15 |
| 323 | AA848795 | Rn.163635; transcribed locus |
| 324 | AI103213 | Rn.164935; transcribed locus |
| 325 | Nans | Rn.17006; N-acetylneuraminic acid synthase (sialic |
| | | acid synthase) (predicted) |
| 326 | BE108415 | Rn.171133; transcribed locus |
| 327 | Pfn2 | Rn.17153; profilin 2 |
| 328 | Ube2n | Rn.177520; ubiguitin-conjugating enzyme E2N |
| 329 | BM384251 | Rn.177573; transcribed locus |
| 330 | Gga2 | Rn.18248; Golgi associated, gamma adaptin ear |
| | | containing, ARF binding protein 2 |
| 331 | BE106888 | Rn.19198; cysteine-rich with EGF-like domains 2 |
| 332 | AI070306 | Rn.19710; transcribed locus |
| 333 | Reln | Rn.198116; reelin |
| 334 | Glp2 | Rn.1998318; interferon, alpha-inducible protein |
| | | (clone IFI-15K) (predicted) |
| 335 | Gpc4 | Rn.19945; glypican 4 |
| 336 | BF567145 | Rn.200155; transcribed locus |
| 337 | Manba | Rn.20578; mannosidase, beta A, lysosomal |
| 338 | BM386110 | Rn.223; proliferating cell nuclear antigen |
| 339 | RGD1562142 | Rn.23219; similar to homeotic protein Hox 2.2 — |
| | | mouse (predicted) |
| 340 | BG378045 | Rn.23614; transcribed locus |
| 341 | AI146051 | Rn.24020; transcribed locus |
| 342 | AI102873 | Rn.2721; transcribed locus |
| 343 | Rdx | Rn.27421; radixin |
| 344 | Dnase 113 | Rn.29996; deoxyribonuclease I-like 3 |
| 345 | Hexb | Rn.3021; hexosaminidase B |
| 346 | Pls3 | Rn.32103; plastin 3 (T-isoform) |
| 347 | RGD1566102_predicted | Rn.34703; transcribed locus |
| 348 | AI535113 | Rn.34745; transcribed locus |
| 349 | Pdia4 | Rn.39305; protein disulfide isomerase associated 4 |
| 350 | AW529628 | Rn.43319; transcribed locus |
| 351 | BI292232 | Rn.43415; transcribed locus |
| 352 | Kcne3 | Rn.44843; potassium voltage-gated channel, Isk- |
| | | related subfamily, member 3 |
| 353 | St14 | Rn.49170; suppression of tumorigenicity 14 (colon |
| | | carcinoma) |
| 354 | Mt1a | Rn.54397; metallothionein 1a |
| 355 | St6gal1 | Rn.54567; betagalactoside alpha 2,6 sialyltransferase 1 |
| 356 | Alcam | Rn.5789; activated leukocyte cell adhesion molecule |
| 357 | Maob | Rn.6656; monoamine oxidase B |
| 358 | AA891161 | Rn.7257; transcribed locus |
| 359 | S1c17a5 | Rn.74591; solute carrier family 17 (anion/sugar |
| | | transporter), member 5 |
| 360 | RGD1306766 | Rn.7655; similar to hypothetical protein FLJ23514 |
| 361 | Gja5 | Rn.88300; gap junction membrane channel protein |
| | | alpha 5 |
| 362 | RGD1566265_predicted | Rn.8881; similar to RIKEN cDNA 2610002M06 |
| | | (predicted) |
| 363 | AI136703 | Rn.92818; transcribed locus |
| 364 | Mta3_predicted | Rn.94848; metastasis associated 3 (predicted) |
| 365 | Pctp | Rn.9487; phosphatidylcholine transfer protein |
| 366 | Map1b | Rn.98152; microtubule-associated protein 1b |
| 367 | Tspan5 | Rn.98240; tetraspanin 5 |
| 368 | Got2 | Rn.98650; glutamate oxaloacetate transaminase 2, |
| | | mitochondrial |
| 369 | BI285489 | Rn.98850; similar to myo-inositol 1-phosphate |
| | | synthase A1 |
| 370 | Zfp423 | Rn.9981; Zinc finger protein 423 |
| 371 | Slc6a6 | Rn.9968; solute carrier family 6 (neurotransmitter |
| | | transporter, taurine), member 6 |
| 372 | Agtr1a | Rn.9814; angiotensin II receptor, type 1 (AT1A) |
| 373 | Ppp1r1a | Rn.9756; protein phosphatase 1, regulatory |
| | | (inhibitor) subunit 1A |
| 374 | Plin | Rn.9737; perilipin |
| 375 | Dgat2 | Rn.9523; diacylglycerol O-acyltransferase homolog |
| | | 2 (mouse) |
| 376 | Pcsk6 | Rn.950; proprotein convertase subtilisin/kexin type 6 |
| 377 | BI281177 | Rn.9403; transcribed locus |
| 378 | AI599621 | Rn.92531; Wilms tumor 1 |
| 379 | Ceacam1 | Rn.91235; CEA-related cell adhesion molecule 1 |
| 380 | Gng11 | Rn.892; guanine nucleotide binding protein (G |
| | | protein), gamma 11 |
| 381 | Cdh11 | Rn.8900; cadherin 11 |
| 382 | Fmo1 | Rn.867; flavin containing monooxygenase 1 |
| 383 | Cbr3_predicted | Rn.8624; carbonyl reductase 3 (predicted) |
| 384 | BE113281 | Rn.85462; quaking homolog, KH domain RNA |
| | | binding (mouse) |
| 385 | Cidea_predicted | Rn.8171; cell death-inducing DNA fragmentation |
| | | factor, alpha subunit-like effector A (predicted) |
| 386 | Cav2 | Rn.81070; caveolin 2 |
| 387 | BI273836 | Rn.79933; transcribed locus |
| 388 | Mmrn2_predicted | Rn.7966; multimerin 2 (predicted) |
| 389 | Agtr1 | Rn.7965; angiotensin receptor-like 1 |
| 390 | Gypc | Rn.7693; Glycophorin C (Gerbich blood group) |
| 391 | RGD1305719_predicted | Rn.76732; similar to putative N-acetyltransferase |
| | | Camello 2 (predicted) |
| 392 | AI171656 | Rn.7615; RGD1564859 (predicted) |
| 393 | Spsb1_predicted | Rn.75037; SplA/ryanodine receptor domain and |
| | | SOCS box containing 1 (predicted) |
| 394 | Bcar3_predicted | Rn.7383; breast cancer anti-estrogen resistance 3 |
| | | (predicted) |
| 395 | BE115406 | Rn.7282; similar to expressed sequence AA408877 |
| 396 | Dlc1 | Rn.7255; deleted in liver cancer 1 |
| 397 | AW915115 | Rn.65477; transcribed locus |
| 398 | Cdkn2c | Rn.63865; cyclin-dependent kinase inhibitor 2C |
| | | (p18, inhibits CDK4) |
| 399 | BF387865 | Rn.63789; Transcribed locus |
| 400 | Tst | Rn.6360; Thiosulfate sulfurtransferase |
| 401 | Mbp | Rn.63285; Myelin basic protein |
| 402 | RGD1311474 | Rn.6288; Similar to transmembrane protein induced |
| | | by tumor necrosis factor alpha |
| 403 | Pfk1 | Rn.59431; Mesoderm specific transcript |
| 404 | BI297693 | Rn.57310; Similar to protein of unknown function |
| | | (predicted) |
| 405 | Agpat2_predicted | Rn.55456; 1-acylglycerol-3-phosphate O- |
| | | acyltransferase 2 (lysophosphatidic acid |
| | | acyltransferase, beta) (predicted) |
| 406 | Ilvb1_predicted | Rn.54315; Synapse defective 1, Rho GTPase, |
| | | homolog 1 (C. elegans) (predicted) |
| 407 | Ptpns1 | Rn.53971; Protein tyrosine phosphatase, non- |
| | | receptor type substrate 1 |
| 408 | Col4a1 | Rn.53801; Procollagen, type IV, alpha 1 |
| 409 | Ccl2 | Rn.4772; Chemokine (C-C motif) ligand 2 |
| 410 | Gprc5b_predicted | Rn.47330; G protein-coupled receptor, family C, |
| | | group 5, member B (predicted) |
| 411 | AI071994 | Rn.44861; Dickkopf homolog 4 (Xenopus laevis) |
| | | (predicted) |
| 412 | BF414285 | Rn.44465; Chemokine-like receptor 1 |
| 413 | Gpd1 | Rn.44452; Glycerol-3-phosphate dehydrogenase 1 |
| | | (soluble) |
| 414 | Acacb | Rn.44359; Transcribed locus |
| 415 | AI412164 | Rn.44086; Transcribed locus |
| 416 | BF283694 | Rn.44024; Transcribed locus |
| 417 | Ankrd5_predicted | Rn.44014; Ankyrin repeat domain 5 (predicted) |
| 418 | AI144739 | Rn.43251; Similar to KIAA0303 (predicted) |
| 419 | BG661061 | Rn.41321; WDNM1 homolog |
| 420 | Prkar2b | Rn.4075; Protein kinase, cAMP dependent |
| | | regulatory, type II beta |
| 421 | BI290794 | Rn.40729; Transcribed locus |
| 422 | BM384701 | Rn.40541; PE responsive protein c64 |
| 423 | RGD1565118_predicted | Rn.39037; Similar to mKIAA0843 protein |
| | | (predicted) |
| 424 | Cd248_predicted | Rn.38806; CD248 antigen, endosialin (predicted) |
| 425 | Acaa2 | Rn.3786; Acetyl-Coenzyme A acyltransferase 2 |
| | | (mitochondrial 3-oxoacyl-Coenzyme A thiolase) |
| 426 | BM390128 | Rn.36545; Tenascin XA |
| 427 | RGD1309578 | Rn.35367; Similar to Aa2-174 |
| 428 | Inhbb | Rn.35074; Inhibin beta-B |
| 429 | AA943681 | Rn.3504; Response gene to complement 32 |
| 430 | BI274428 | Rn.34454; Transcribed locus |
| 431 | Gpm6a | Rn.34370; Glycoprotein m6a |
| 432 | Cbr1 | Rn.3425; Carbonyl reductase 1 |
| 433 | Slc1a3 | Rn.34134; Solute carrier family 1 (glial high affinity |
| | | glutamate transporter), member 3 |
| 434 | AI179450 | Rn.34019; Transcribed locus |
| 435 | RGD1560062_predicted | Rn.32891; Similar to Laminin alpha-4 chain |
| | | precursor (predicted) |
| 436 | Phyhd1 | Rn.32623; Phytanoyl-CoA dioxygenase domain |
| | | containing 1 |
| 437 | Rgl1_predicted | Rn.28005; Ral guanine nucleotide dissociation |
| | | stimulator, -like 1 (predicted) |
| 438 | Grifin | Rn.26894; Galectin-related inter-fiber protein |
| 439 | BG381647 | Rn.26832; Transcribed locus |
| 440 | Ccl7 | Rn.26815; Chemokine (C-C motif) ligand 7 |
| 441 | AI548615 | Rn.26537; Transcribed locus |
| 442 | Per2 | Rn.25935; Period homolog 2 (Drosophila) |
| 443 | Dgat1 | Rn.252; Diacylglycerol O-acyltransferase 1 |
| 444 | Gda | Rn.24783; Transcribed locus |
| 445 | Psme1 | Rn.2472; Proteasome (prosome, macropain) 28 |
| | | subunit, alpha |
| 446 | Tm4sf1_predicted | Rn.24712; Transmembrane 4 superfamily member 1 |
| | | (predicted) |
| 447 | Slc22a3 | Rn.24231; Solute carrier family 22, member 3 |
| 448 | AI228291 | Rn.2361; Similar to CG3740-PA |
| 449 | Rasip1_predicted | Rn.23451; Ras interacting protein 1 (predicted) |
| 450 | Pparg | Rn.23443; Peroxisome proliferator activated receptor |
| | | gamma |
| 451 | BG378238 | Rn.23273; Transcribed locus |
| 452 | Abca8a_predicted | Rn.22789; ATP-binding cassette, sub-family A |
| | | (ABC1), member 8a (predicted) |
| 453 | BF290937 | Rn.22733; Transcribed locus |
| 454 | Sox18 | Rn.22446; SRY-box containing gene 18 |
| 455 | AI230554 | Rn.22441; Carbonic anhydrase VB, mitochondrial |
| 456 | Col4a2_predicted | Rn.2237; Procollagen, type IV, alpha 2 (predicted) |
| 457 | BF547294 | Rn.22135; Protein tyrosine phosphatase, receptor |
| | | type, M |
| 458 | Id1 | Rn.2113; Inhibitor of DNA binding 1 |
| 459 | Sulf1 | Rn.20664; Transcribed locus |
| 460 | AI411941 | Rn.20633; Fibronectin type III domain containing 1 |
| 461 | AI385260 | Rn.20514; Unknown (protein for MGC: 72614) |
| 462 | RGD1562428_predicted | Rn.199567; Transcribed locus |
| 463 | Aoc3 | Rn.198327; Amine oxidase, copper containing 3 |
| 464 | AI599365 | Rn.19608; Transcribed locus |
| 465 | RGD1305061 | Rn.196026; Similar to RIKEN cDNA 2700055K07 |
| 466 | BF282889 | Rn.19393; Transcribed locus |
| 467 | RGD1311800 | Rn.1935; Similar to genethonin 1 |
| 468 | Daf1 | Rn.18841; decay accelerating factor 1 |
| 469 | AI030806 | Rn.18599; Transcribed locus |
| 470 | BM386662 | Rn.18571; Tumor suppressor candidate 5 |
| 471 | BF283405 | Rn.18479; Transcribed locus |
| 472 | BI277619 | Rn.18388; Transcribed locus |
| 473 | Anxa1 | Rn.1792; Annexin A1 |
| 474 | Phlda3 | Rn.17905; Pleckstrin homology-like domain, family |
| | | A, member 3 |
| 475 | Zdhhc2 | Rn.17310; Zinc finger, DHHC domain containin 2 |
| 476 | AI101500 | Rn.17209; Transcribed locus |
| 477 | AW525722 | Rn.168623; Transcribed locus Transcribed locus |
| 478 | AI600020 | Rn.168403; Transcribed locus |
| 479 | Hdgfrp2 | Rn.167154; Transcribed locus |
| 480 | Degs1 | Rn.167052; Transcribed locus |
| 481 | BM389225 | Rn.1664; Transcribed locus |
| 482 | AI407050 | Rn.165854; Transcribed locus |
| 483 | BF291140 | Rn.165854; Transcribed locus |
| 484 | AI176379 | Rn.165711; Transcribed locus |
| 485 | BF403558 | Rn.165637; Transcribed locus |
| 486 | AI008140 | Rn.165579; Transcribed locus |
| 487 | AW536030 | Rn.165356; Similar to liver-specific bHLH-Zip |
| | | transcription factor |
| 488 | Sdpr | Rn.165134; Transcribed locus |
| 489 | AI385201 | Rn.164647; Transcribed locus |
| 490 | Tgfbr2 | Rn.164421; Transcribed locus |
| 491 | AW535515 | Rn.164403; Transcribed locus |
| 492 | Gata6 | Rn.164357; Transcribed locus |
| 493 | RGD1566234_predicted | Rn.164243; Transcribed locus |
| 494 | Acaca | Rn.163753; Acetyl-coenzyme A carboxylase alpha |
| 495 | RGD1311037 | Rn.163715; Transcribed locus |
| 496 | AA926305 | Rn.163580; Transcribed locus |
| 497 | Efemp1 | Rn.163265; Epidermal growth factor-containing |
| | | fibulin-like extracellular matrix protein 1 |
| 498 | Asp | Rn.163202; Adaptor protein with pleckstrin |
| | Aps | homology and src homology 2 domains |
| 499 | Vnn1 | Rn.16319; Vanin 1 |
| 500 | Lpin1 | Rn.162853; Lipin 1 |
| 501 | Ppp1r3c | Rn.162528; Protein phosphatase 1, regulatory |
| | | (inhibitor) subunit 3C |
| 502 | Twist1 | Rn.161904; Twist gene homolog 1 (Drosophila) |
| 503 | C6 | Rn.16145; Complement component 6 |
| 504 | Cabc1 | Rn.160865; Chaperone, ABC1 activity of bc1 |
| | | complex like (S. pombe) |
| 505 | Vegfb | Rn.160277; Transcribed locus |
| 506 | Ehd2 | Rn.16016; EH-domain containing 2 |
| 507 | Dpyd | Rn.158382; Dihydropyrimidine dehydrogenase |
| 508 | Nnmt_predicted | Rn.15755; Nicotinamide N-methyltransferase |
| | | (predicted) |
| 509 | BI289692 | Rn.15749; Transcribed locus |
| 510 | Chpt1 | Rn.154718; Choline phosphotransferase 1 |
| 511 | BI295900 | Rn.15413; Dihydrolipoamide S-acetyltransferase (E2 |
| | | component of pyruvate dehydrogenase complex) |
| 512 | AW917217 | Rn.153603; CCAAT/enhancer binding protein |
| | | (C/EBP), alpha |
| 513 | AA942745 | Rn.149118; Transcribed locus |
| 514 | BI283648 | Rn.148951; Hypothetical protein LOC691485 |
| 515 | BF393275 | Rn.148773; Transcribed locus |
| 516 | AI555775 | Rn.147356; Transcribed locus |
| 517 | Tgif | Rn.144418; Transcribed locus |
| 518 | Cldn15_predicted | Rn.144007; Transcribed locus |
| 519 | AI578098 | Rn.137828; Similar to CD209 antigen |
| 520 | Cyp2e1 | Rn.1372; Cytochrome P450, family 2, subfamily e, |
| | | polypeptide 1 |
| 521 | Tm4sf2_mapped | Rn.13685; Transmembrane 4 superfamily member 2 |
| | | (mapped) |
| 522 | Mdh1 | Rn.13492; Malate dehydrogenase 1, NAD (soluble) |
| 523 | Slc2a4 | Rn.1314; Solute carrier family 2 (facilitated glucose |
| | | transporter), member 4 |
| 524 | Cmkor1 | Rn.12959; Chemokine orphan receptor 1 |
| 525 | AW528864 | Rn.129539; Transcribed locus |
| 526 | Dnd1 | Rn.12947; Similar to KIAA0564 protein (predicted) |
| 527 | AW528112 | Rn.119594; Transcribed locus |
| 528 | BF397229 | Rn.11817; Transcribed locus |
| 529 | Sfxn1 | Rn.115752; Sideroflexin 1 |
| 530 | Hrasls3 | Rn.11377; HRAS like suppressor 3 |
| 531 | Pla2g2a | Rn.11346; Phospholipase A2, group IIA (platelets, |
| | | synovial fluid) |
| 532 | Ebf1 | Rn.11257; Early B-cell factor 1 |
| 533 | Sdc2 | Rn.11127; Syndecan 2 |
| 534 | Aqp7 | Rn.11111; Aquaporin 7 |
| 535 | Pc | Rn.11094; Pyruvate carboxylase |
| 536 | Bhlhb3 | Rn.10784; Basic helix-loop-helix domain containing, |
| | | class B3 |
| 537 | AI602542 | Rn.107412; Transcribed locus |
| 538 | Maf | Rn.10726; V-maf musculoaponeurotic fibrosarcoma |
| | | oncogene homolog (avian) |
| 539 | Cpa3 | Rn.10700; Carboxypeptidase A3 |
| 540 | Mcpt1 | Rn.10698; Mast cell protease 1 |
| 541 | RGD1309821_predicted | Rn.106115; Similar to KIAA1161 protein (predicted) |
| 542 | Acvr1c | Rn.10580; Activin A receptor, type IC |
| 543 | Ppp2r5a_predicted | Rn.104461; Protein phosphatase 2, regulatory |
| | | subunit B (B56), alpha isoform (predicted) |
| 544 | Pde3b | Rn.10322; Phosphodiesterase 3B |
| 545 | Pxmp2 | Rn.10292; Peroxisomal membrane protein 2 |
| 546 | P2rx5 | Rn.10257; Purinergic receptor P2X, ligand-gated ion |
| | | channel, 5 |
| 547 | Cma1 | Rn.10182; Chymase 1, mast cell |
| 548 | Pfkfb1 | Rn.10115; 6-phosphofructo-2-kinase/fructose-2,6- |
| | | biphosphatase 1 |

Levels of the T2DBMARKERS can be determined at the protein or nucleic acid level using any method known in the art. T2DBMARKER amounts can be detected, inter alia, electrophoretically (such as by agarose gel electrophoresis, sodium dodecyl sulfate-polyacrylamide gel electrophoresis (SDS-PAGE), Tris-HCl polyacrylamide gels, non-denaturing protein gels, two-dimensional gel electrophoresis (2DE), and the like), immunochemically (i.e., radioimmunoassay, immunoblotting, immunoprecipitation, immunofluorescence, enzyme-linked immunosorbent assay), by “proteomics technology”, or by “genomic analysis.” For example, at the nucleic acid level, Northern and Southern hybridization analysis, as well as ribonuclease protection assays using probes which specifically recognize one or more of these sequences can be used to determine gene expression. Alternatively, expression can be measured using reverse-transcription-based PCR assays (RT-PCR), e.g., using primers specific for the differentially expressed sequence of genes. Expression can also be determined at the protein level, e.g., by measuring the levels of peptides encoded by the gene products described herein, or activities thereof. Such methods are well known in the art and include, e.g., immunoassays based on antibodies to proteins encoded by the genes, aptamers or molecular imprints. Any biological material can be used for the detection/quantification of the protein or its activity. Alternatively, a suitable method can be selected to determine the activity of proteins encoded by the marker genes according to the activity of each protein analyzed.

“Proteomics technology” includes, but is not limited to, surface enhanced laser desorption ionization (SELDI), matrix-assisted laser desorption ionization-time of flight (MALDI-TOF), high performance liquid chromatography (HPLC), liquid chromatography with or without mass spectrometry (LC/MS), tandem LC/MS, protein arrays, peptide arrays, and antibody arrays.

“Genome analysis” can comprise, for example, polymerase chain reaction (PCR), real-time PCR (such as by Light Cycler®, available from Roche Applied Sciences), serial analysis of gene expression (SAGE), Northern blot analysis, and Southern blot analysis.

Microarray technology can be used as a tool for analyzing gene or protein expression, comprising a small membrane or solid support (such as but not limited to microscope glass slides, plastic supports, silicon chips or wafers with or without fiber optic detection means, and membranes including nitrocellulose, nylon, or polyvinylidene fluoride). The solid support can be chemically (such as silanes, streptavidin, and numerous other examples) or physically derivatized (for example, photolithography) to enable binding of the analyte of interest, usually nucleic acids, proteins, or metabolites or fragments thereof. The nucleic acid or protein can be printed (i.e., inkjet printing), spotted, or synthesized in situ. Deposition of the nucleic acid or protein of interest can be achieved by xyz robotic microarrayers, which utilize automated spotting devices with very precise movement controls on the x-, y-, and z-axes, in combination with pin technology to provide accurate, reproducible spots on the arrays. The analytes of interest are placed on the solid support in an orderly or fixed arrangement so as to facilitate easy identification of a particularly desired analyte. A number of microarray formats are commercially available from, inter alia, Affymetrix, ArrayIt, Agilent Technologies, Asper Biotech, BioMicro, CombiMatrix, GenePix, Nanogen, and Roche Diagnostics.

The nucleic acid or protein of interest can be synthesized in the presence of nucleotides or amino acids tagged with one or more detectable labels. Such labels include, for example, fluorescent dyes and chemiluminescent labels. In particular, for microarray detection, fluorescent dyes such as but not limited to rhodamine, fluorescein, phycoerythrin, cyanine dyes like Cy3 and Cy5, and conjugates like streptavidin-phycoerythrin (when nucleic acids or proteins are tagged with biotin) are frequently used. Detection of fluorescent signals and image acquisition are typically achieved using confocal fluorescence laser scanning or photomultiplier tube, which provide relative signal intensities and ratios of analyte abundance for the nucleic acids or proteins represented on the array. A wide variety of different scanning instruments are available, and a number of image acquisition and quantification packages are associated with them, which allow for numerical evaluation of combined selection criteria to define optimal scanning conditions, such as median value, inter-quartile range (IQR), count of saturated spots, and linear regression between pair-wise scans (r² and P). Reproducibility of the scans, as well as optimization of scanning conditions, background correction, and normalization, are assessed prior to data analysis.

Normalization refers to a collection of processes that are used to adjust data means or variances for effects resulting from systematic non-biological differences between arrays, subarrays (or print-tip groups), and dye-label channels. An array is defined as the entire set of target probes on the chip or solid support. A subarray or print-tip group refers to a subset of those target probes deposited by the same print-tip, which can be identified as distinct, smaller arrays of proves within the full array. The dye-label channel refers to the fluorescence frequency of the target sample hybridized to the chip. Experiments where two differently dye-labeled samples are mixed and hybridized to the same chip are referred to in the art as “dual-dye experiments”, which result in a relative, rather than absolute, expression value for each target on the array, often represented as the log of the ratio between “red” channel and “green channel.” Normalization can be performed according to ratiometric or absolute value methods. Ratiometric analyses are mainly employed in dual-dye experiments where one channel or array is considered in relation to a common reference. A ratio of expression for each target probe is calculated between test and reference sample, followed by a transformation of the ratio into log₂(ratio) to symmetrically represent relative changes. Absolute value methods are used frequently in single-dye experiments or dual-dye experiments where there is no suitable reference for a channel or array. Relevant “hits” are defined as expression levels or amounts that characterize a specific experimental condition. Usually, these are nucleic acids or proteins in which the expression levels differ significantly between different experimental conditions, usually by comparison of the expression levels of a nucleic acid or protein in the different conditions and analyzing the relative expression (“fold change”) of the nucleic acid or protein and the ratio of its expression level in one set of samples to its expression in another set.

Data obtained from microarray experiments can be analyzed by any one of numerous statistical analyses, such as clustering methods and scoring methods. Clustering methods attempt to identify targets (such as nucleic acids and/or proteins) that behave similarly across a range of conditions or samples. The motivation to find such targets is driven by the assumption that targets that demonstrate similar patterns of expression share common characteristics, such as common regulatory elements, common functions, or common cellular origins.

Hierarchical clustering is an agglomerative process in which single-member clusters are fused to bigger and bigger clusters. The procedure begins by computing a pairwise distance matrix between all the target molecules, the distance matrix is explored for the nearest genes, and they are defined as a cluster. After a new cluster is formed by agglomeration of two clusters, the distance matrix is updated to reflect its distance from all other clusters. Then, the procedure searches for the nearest pair of clusters to agglomerate, and so on. This procedure results in a hierarchical dendrogram in which multiple clusters are fused to nodes according to their similarity, resulting in a single hierarchical tree. Hierarchical clustering software algorithms include Cluster and Treeview.

K-means clustering is an iterative procedure that searches for clusters that are defined in terms of their “center” points or means. Once a set of cluster centers is defined, each target molecule is assigned to the cluster it is closest to. The clustering algorithm then adjusts the center of each cluster of genes to minimize the sum of distances of target molecules in each cluster to the center. This results in a new choice of cluster centers, and target molecules can be reassigned to clusters. These iterations are applied until convergence is observed. Self-organizing maps (SOMs) are related in part to the k-means procedure, in that the data is assigned to a predetermined set of clusters. However, unlike k-means, what follows is an iterative process in which gene expression vectors in each cluster are “trained” to find the best distinctions between the different clusters. In other words, a partial structure is imposed on the data and then this structure is iteratively modified according to the data. SOM is included in many software packages, such as, for instance, GeneCluster. Other clustering methods include graph-theoretic clustering, which utilizes graph-theoretic and statistical techniques to identify tight groups of highly similar elements (kernels), which are likely to belong to the same true cluster. Several heuristic procedures are then used to expand the kernels into the full clustering. An example of software utilizing graph-theoretic clustering includes CLICK in combination with the Expander visualization tool.

Data obtained from high-throughput expression analyses can be scored using statistical methods such as parametric and non-parametric methods. Parametric approaches model expression profiles within a parametric representation and ask how different the parameters of the experimental groups are. Examples of parametric methods include, without limitation, t-tests, separation scores, and Bayesian t-tests. Non-parametric methods involve analysis of the data, wherein no a priori assumptions are made about the distribution of expression profiles in the data, and the degree to which the two groups of expression measurements are distinguished is directly examined. Another method uses the TNOM, or the threshold number of misclassifications, which measures the success in separation two groups of samples by a simple threshold over the expression values.

SAGE (serial analysis of gene expression) can also be used to systematically determine the levels of gene expression. In SAGE, short sequence tags within a defined position containing sufficient information to uniquely identify a transcript are used, followed by concatenation of tags in a serial fashion. See, for example, Velculescu V. E. et al, (1995) Science 270: 484-487. Polyadenylated RNA is isolated by oligo-dT priming, and cDNA is then synthesized using a biotin-labeled primer. The cDNA is subsequently cleaved with an anchoring restriction endonucleases, and the 3′-terminal cDNA fragments are bound to streptavidin-coated beads. An oligonucleotide linker containing recognition sites for a tagging enzyme is linked to the bound cDNA. The tagging enzyme can be a class II restriction endonucleases that cleaves the DNA at a constant number of bases 3′ to the recognition site, resulting in the release of a short tag and the linker from the beads after digestion with the enzyme. The 3′ ends of the released tags plus linkers are then blunt-ended and ligated to one another to form linked ditags that are approximately 100 base pairs in length. The ditags are then subjected to PCR amplification, after which the linkers and tags are released by digestion with the anchoring restriction endonucleases. Thereafter, the tags (usually ranging in size from 25-30-mers) are gel purified, concatenated, and cloned into a sequence vector. Sequencing the concatemers enables individual tags to be identified and the abundance of the transcripts for a given cell or tissue type can be determined.

The T2DBMARKER proteins, polypeptides, mutations, and polymorphisms thereof can be detected in any manner known to those skilled in the art. Of particular utility are two-dimensional gel electrophoresis, which separates a mixture of proteins (such as found in biological samples such as serum) in one dimension according to the isoelectric point (such as, for example, a pH range from 5-8), and according to molecular weight in a second dimension. Two-dimensional liquid chromatography is also advantageously used to identify or detect T2DBMARKER proteins, polypeptides, mutations, and polymorphisms of the invention, and one specific example, the ProteomeLab PF 2D Protein Fractionation System is detailed in the Examples. The PF 2D system resolves proteins in one dimension by isoelectric point and by hydrophobicity in the second dimension. Another advantageous method of detecting proteins, polypeptides, mutations, and polymorphisms include SELDI (disclosed herein) and other high-throughput proteomic arrays.

T2DBMARKER proteins, polypeptides, mutations, and polymorphisms can be typically detected by contacting a sample from the subject with an antibody which binds the T2DBMARKER protein, polypeptide, mutation, or polymorphism and then detecting the presence or absence of a reaction product. The antibody may be monoclonal, polyclonal, chimeric, or a fragment of the foregoing, as discussed in detail herein, and the step of detecting the reaction product may be carried out with any suitable immunoassay. In a particularly preferred embodiment, the T2DBMARKER proteins, polypeptides, mutations, and polymorphisms can be detected with an isolated antibody of the present invention, as disclosed elsewhere in this disclosure. The isolated antibody provided by the invention can comprise, for example, a human constant region (as defined herein) and an antigen-binding region that binds to one or more T2DBMARKERS set forth in Table 1, preferably at least one, preferably two, three, four, five, six, seven, eight, nine, ten or more amino acid residues of SEQ ID NO: 1. The sample from the subject is typically a biological fluid as described above, and may be the same sample of biological fluid used to conduct the method described above.

Immunoassays carried out in accordance with the present invention may be homogeneous assays or heterogeneous assays. In a homogeneous assay, the immunological reaction usually involves the specific antibody (e.g., anti-T2 DBMARKER protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A agarose, protein G agarose, latex, polystyrene, magnetic or paramagnetic beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are oligonucleotides, immunoblotting, immunoprecipitation, immunofluorescence methods, chemiluminescence methods, electrochemiluminescence or enzyme-linked immunoassays.

Those skilled in the art will be familiar with numerous specific immunoassay formats and variations thereof which may be useful for carrying out the method disclosed herein. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogenous Specific Binding Assay Employing a Coenzyme as Label.”

Antibodies, such as those provided by the present invention, can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein) in accordance with known techniques.

Antibodies can also be useful for detecting post-translational modifications of T2DBMARKER proteins, polypeptides, mutations, and polymorphisms, such as tyrosine phosphorylation, threonine phosphorylation, serine phosphorylation, glycosylation (e.g., O-GlcNAc). Such antibodies specifically detect the phosphorylated amino acids in a protein or proteins of interest, and can be used in immunoblotting, immunofluorescence, and ELISA assays described herein. These antibodies are well-known to those skilled in the art, and commercially available. Post-translational modifications can also be determined using metastable ions in reflector matrix-assisted laser desorption ionization-time of flight mass spectrometry (MALDI-TOF) (Wirth, U. et al. (2002) Proteomics 2(10): 1445-51).

For T2DBMARKER proteins, polypeptides, mutations, and polymorphisms known to have enzymatic activity, the activities can be determined in vitro using enzyme assays known in the art. Such assays include, without limitation, kinase assays, phosphatase assays, reductase assays, among many others. Modulation of the kinetics of enzyme activities can be determined by measuring the rate constant KM using known algorithms, such as the Hill plot, Michaelis-Menten equation, linear regression plots such as Lineweaver-Burk analysis, and Scatchard plot.

Using sequence information provided by the database entries for the T2DBMARKER sequences, expression of the T2DBMARKER sequences can be detected (if present) and measured using techniques well known to one of ordinary skill in the art. For example, sequences within the sequence database entries corresponding to T2DBMARKER sequences, or within the sequences disclosed herein, can be used to construct probes for detecting T2DBMARKER RNA sequences in, e.g., Northern blot hybridization analyses or methods which specifically, and, preferably, quantitatively amplify specific nucleic acid sequences. As another example, the sequences can be used to construct primers for specifically amplifying the T2DBMARKER sequences in, e.g., amplification-based detection methods such as reverse-transcription based polymerase chain reaction (RT-PCR). When alterations in gene expression are associated with gene amplification, deletion, polymorphisms, and mutations, sequence comparisons in test and reference populations can be made by comparing relative amounts of the examined DNA sequences in the test and reference cell populations.

Expression of the genes disclosed herein can be measured at the RNA level using any method known in the art. For example, Northern hybridization analysis using probes which specifically recognize one or more of these sequences can be used to determine gene expression. Alternatively, expression can be measured using reverse-transcription-based PCR assays (RT-PCR), e.g., using primers specific for the differentially expressed sequences.

Alternatively, T2DBMARKER protein and nucleic acid metabolites or fragments can be measured. The term “metabolite” includes any chemical or biochemical product of a metabolic process, such as any compound produced by the processing, cleavage or consumption of a biological molecule (e.g., a protein, nucleic acid, carbohydrate, or lipid). Metabolites can be detected in a variety of ways known to one of skill in the art, including the refractive index spectroscopy (RI), ultra-violet spectroscopy (UV), fluorescence analysis, radiochemical analysis, near-infrared spectroscopy (near-IR), nuclear magnetic resonance spectroscopy (NMR), light scattering analysis (LS), mass spectrometry, pyrolysis mass spectrometry, nephelometry, dispersive Raman spectroscopy, gas chromatography combined with mass spectrometry, liquid chromatography combined with mass spectrometry, matrix-assisted laser desorption ionization-time of flight (MALDI-TOF) combined with mass spectrometry, surface-enhanced laser desorption ionization (SELDI), ion spray spectroscopy combined with mass spectrometry, capillary electrophoresis, NMR and IR detection. (See, WO 04/056456 and WO 04/088309, each of which are hereby incorporated by reference in their entireties) In this regard, other T2DBMARKER analytes can be measured using the above-mentioned detection methods, or other methods known to the skilled artisan.

### Kits

The invention also includes a T2DBMARKER-detection reagent, e.g., nucleic acids that specifically identify one or more T2DBMARKER nucleic acids by having homologous nucleic acid sequences, such as oligonucleotide sequences, complementary to a portion of the T2DBMARKER nucleic acids or antibodies to proteins encoded by the T2DBMARKER nucleic acids packaged together in the form of a kit. The kits of the present invention allow one of skill in the art to generate the reference and subject expression profiles disclosed herein. The kits of the invention can also be used to advantageously carry out any of the methods provided in this disclosure. The oligonucleotides can be fragments of the T2DBMARKER genes. For example the oligonucleotides can be 200, 150, 100, 50, 25, 10 or less nucleotides in length. The T2DBMARKER-detection reagents can also comprise, inter alia, antibodies or fragments of antibodies, and aptamers. The kit may contain in separate containers a nucleic acid or antibody (either already bound to a solid matrix or packaged separately with reagents for binding them to the matrix), control formulations (positive and/or negative), and/or a detectable label. Instructions (e.g., written, tape, VCR, CD-ROM, etc.) for carrying out the assay detecting one or more T2DBMARKERS of the invention may be included in the kit. The assay may for example be in the form of a Northern blot hybridization or a sandwich ELISA as known in the art. Alternatively, the kit can be in the form of a microarray as known in the art.

Diagnostic kits for carrying out the methods described herein are produced in a number of ways. Preferably, the kits of the present invention comprise a control (or reference) sample derived from a subject having normal glucose levels. Alternatively, the kits can comprise a control sample derived from a subject who has been diagnosed with or identified as suffering from type 2 Diabetes or a pre-diabetic condition. In one embodiment, the diagnostic kit comprises (a) an antibody (e.g., fibrinogen αC domain peptide) conjugated to a solid support and (b) a second antibody of the invention conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. Alternatively, a test kit contains (a) an antibody of the invention, and (b) a specific binding partner for the antibody conjugated to a detectable group. The test kit may be packaged in any suitable manner, typically with all elements in a single container, optionally with a sheet of printed instructions for carrying out the test.

For example, T2DBMARKER detection reagents can be immobilized on a solid matrix such as a porous strip to form at least one T2DBMARKER detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, e.g., a higher amount in the first detection site and lesser amounts in subsequent sites. Upon the addition of test sample, the number of sites displaying a detectable signal provides a quantitative indication of the amount of T2DBMARKERS present in the sample. The detection sites may be configured in any suitably detectable shape and are typically in the shape of a bar or dot spanning the width of a test strip.

Alternatively, the kit contains a nucleic acid substrate array comprising one or more nucleic acid sequences. The nucleic acids on the array specifically identify one or more nucleic acid sequences represented by T2DBMARKERS 1-548. In various embodiments, the expression of 2, 3, 4, 5, 6, 7, 8, 9, 10, 15, 20, 25, 30, 40, 50, or more of the T2DBMARKERS 1-548 can be identified by virtue of binding to the array. The substrate array can be on, e.g., a solid substrate, e.g., a “chip” as described in U.S. Pat. No. 5,744,305. Alternatively, the substrate array can be a solution array, e.g., xMAP (Luminex, Austin, Tex.), Cyvera (Illumina, San Diego, Calif.), CellCard (Vitra Bioscience, Mountain View, Calif.) and Quantum Dots' Mosaic (Invitrogen, Carlsbad, Calif.).

The skilled artisan can routinely make antibodies, nucleic acid probes, e.g., oligonucleotides, aptamers, siRNAs, antisense oligonucleotides, against any of the T2DBMARKERS in Table 1. The Examples presented herein describe generation of monoclonal antibodies in mice, as well as generation of polyclonal hyperimmune serum from rabbits. Such techniques are well-known to those of ordinary skill in the art.

### Antibodies

The present invention also provides antibodies that are capable of binding to one or more T2DBMARKERS presented in Table 1, and preferably, antibodies that are capable of binding to one or more amino acids of SEQ ID NO: 1. The term “antibody” as used in the context of the present invention includes polyclonal antibodies, monoclonal antibodies (mAbs), chimeric antibodies, anti-idiotypic (anti-Id) antibodies, that can be labeled in soluble or bound form, as well as fragments, regions, or derivatives thereof, provided by any known technique, such as, but not limited to, enzymatic cleavage, peptide synthesis, or recombinant techniques.

Polyclonal antibodies are heterogeneous populations of antibody molecules derived from the sera of animals immunized with an antigen. A monoclonal antibody contains a substantially homogeneous population of antibodies specific to antigens, which population contains substantially similar epitope binding sites. MAbs may be obtained by methods known to those skilled in the art. See, for example Kohler and Milstein, Nature 256:495-497 (1975); U.S. Pat. No. 4,376,110; Ausubel et al., eds., Current Protocols in Molecular Biology, Greene Publishing Assoc. and Wiley Interscience, N.Y., (1987, 1992); and Harlow and Lane ANTIBODIES. A Laboratory Manual Cold Spring Harbor Laboratory (1988); Colligan et al., eds., Current Protocols in Immunology, Greene Publishing Assoc. and Wiley Interscience, N.Y., (1992, 1993), the contents of which references are incorporated entirely herein by reference.

Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of mAbs in vivo or in situ makes this a preferred method of production.

Chimeric antibodies are molecules different portions of which are derived from different animal species, such as those having variable region derived from a murine mAb and a human immunoglobulin constant region, which are primarily used to reduce immunogenicity in application and to increase yields in production, for example, where murine mabs have higher yields from hybridomas but higher immunogenicity in humans, such that human/murine chimeric mAbs are used. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 86/01533, (published Mar. 13, 1986); Kudo et al., European Patent Application 184187 (published Jun. 11, 1986); Morrison et al., European Patent Application 173494 (published Mar. 5, 1986); Sahagan et al., J. Immunol. 137:1066-1074 (1986); Robinson et al., International Patent Publication No. PCT/US86/02269 (published 7 May 1987); Liu et al., Proc. Natl. Acad. Sci. USA 84:3439-3443 (1987); Sun et al., Proc. Natl. Acad. Sci. USA 84:214-218 (1987); Better et al., Science 240:1041-1043 (1988); and Harlow and Lane Antibodies: a Laboratory Manual Cold Spring Harbor Laboratory (1988)). These references are entirely incorporated herein by reference.

An anti-idiotypic (anti-Id) antibody is an antibody which recognizes unique determinants generally associated with the antigen-binding site of an antibody. An Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference.

The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

Antibodies of the present invention can include at least one of a heavy chain constant region (H_{c}), a heavy chain variable region (Hᵥ), a light chain variable region (Lᵥ) and a light chain constant region (L_{c}), wherein a polyclonal Ab, monoclonal Ab, fragment and/or regions thereof include at least one heavy chain variable region (Hᵥ) or light chain variable region (Lᵥ) which binds a portion of SEQ ID NO: 1. Preferred methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference.

The techniques to raise antibodies of the present invention to small peptide sequences that recognize and bind to those sequences in the free or conjugated form or when presented as a native sequence in the context of a large protein are well known in the art. Such antibodies include murine, murine-human and human-human antibodies produced by hybridoma or recombinant techniques known in the art.

As used herein, the term “antigen binding region” refers to that portion of an antibody molecule which contains the amino acid residues that interact with an antigen and confer on the antibody its specificity and affinity for the antigen. The antibody region includes the “framework” amino acid residues necessary to maintain the proper conformation of the antigen-binding residues.

As used herein, the term “chimeric antibody” includes monovalent, divalent or polyvalent immunoglobulins. A monovalent chimeric antibody is a dimer (HL) formed by a chimeric H chain associated through disulfide bridges with a chimeric L chain. A divalent chieric antibody is tetramer (H₂L₂) formed by two HL dimers associated through at least one disulfide bridge. A polyvalent chimeric antibody can also be produced, for example, by employing a C_{H} region that aggregates (e.g., from an IgM H chain, or μ chain).

Murine and chimeric antibodies, fragments and regions of the present invention comprise individual heavy (H) and/or light (L) immunoglobulin chains. A chimeric H chain comprises an antigen binding region derived from the H chain of a non-human antibody specific for one or more T2DBMARKERS or preferably, SEQ ID NO: 1, which is linked to at least a portion of a human H chain C region (C_{H}), such as CH₁ or CH₂.

A chimeric L chain according to the present invention, comprises an antigen binding region derived from the L chain of a non-human antibody specific for one or more T2DBMARKERS or preferably, SEQ ID NO: 1, linked to at least a portion of a human L chain C region (C_{L}). Antibodies, fragments or derivatives having chimeric H chains and L chains of the same or different variable region binding specificity, can also be prepared by appropriate association of the individual polypeptide chains, according to known method steps, e.g., according to Ausubel, Harlow, and Colligan, the contents of which references are incorporated entirely herein by reference. With this approach, hosts expressing chimeric H chains (or their derivatives) are separately cultured from hosts expressing chimeric L chains (or their derivatives), and the immunoglobulin chains are separately recovered and then associated. Alternatively, the hosts can be co-cultured and the chains allowed to associate spontaneously in the culture medium, followed by recovery of the assembled immunoglobulin, fragment or derivative.

The hybrid cells are formed by the fusion of a non-human anti-T2DBMARKER or anti-SEQ ID NO: 1 (e.g., anti-D3 as disclosed in the Examples) antibody-producing cell, typically a spleen cell of an animal immunized against either natural or recombinant T2DBMARKERS or SEQ ID NO: 1, or a peptide fragment of any one or more of the T2DBMARKERS or SEQ ID NO:1. Alternatively, the non-human antibody-producing cell can be a B lymphocyte obtained from the blood, spleen, lymph nodes or other tissue of an animal immunized with one or more T2DBMARKERS, or the full or partial amino acid sequence of SEQ ID NO: 1.

The second fusion partner, which provides the immortalizing function, can be a lymphoblastoid cell or a plasmacytoma or myeloma cell, which is not itself an antibody producing cell, but is malignant. Preferred fusion partner cells include the hybridoma SP2/0-Ag14, abbreviated as SP2/0 (ATCC CRL1581) and the myeloma P3X63Ag8 (ATCC TIB9), or its derivatives. See, e.g, Ausubel, Harlow, and Colligan, the contents of which are incorporated entirely herein by reference.

The antibody-producing cell contributing the nucleotide sequences encoding the antigen-binding region of the chimeric antibody of the present invention can also be produced by transformation of a non-human, such as a primate, or a human cell. For example, a B lymphocyte which produces an antibody of the invention can be infected and transformed with a virus such as Epstein-Barr virus to yield an immortal antibody producing cell (Kozbor et al., Immunol. Today 4:72-79 (1983)). Alternatively, the B lymphocyte can be transformed by providing a transforming gene or transforming gene product, as is well-known in the art. See, e.g, Ausubel infra, Harlow infra, and Colligan infra, the contents of which references are incorporated entirely herein by reference.

Monoclonal antibodies obtained by cell fusions and hybridomas are accomplished by standard procedures well known to those skilled in the field of immunology. Fusion partner cell lines and methods for fusing and selecting hybridomas and screening for mAbs are well known in the art. See, e.g, Ausubel, Harlow, and Colligan, the contents of which are incorporated entirely herein by reference.

The mAbs of the present invention can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

The invention also provides for “derivatives” of the murine or chimeric antibodies, fragments, regions or derivatives thereof, which term includes those proteins encoded by truncated or modified genes to yield molecular species functionally resembling the immunoglobulin fragments. The modifications include, but are not limited to, addition of genetic sequences coding for cytotoxic proteins such as plant and bacterial toxins. The fragments and derivatives can be produced from any of the hosts of this invention. Alternatively, antibodies, fragments and regions can be bound to cytotoxic proteins or compounds in vitro, to provide cytotoxic antibodies which would selectively kill cells having receptors corresponding to one or more T2DBMARKERS.

Fragments include, for example, Fab, Fab′, F(ab′)₂ and Fv. These fragments lack the Fc fragment of intact antibody, clear more rapidly from the circulation, and can have less non-specific tissue binding than an intact antibody (Wahl et al., J. Nucl. Med. 24:316-325 (1983)). These fragments are produced from intact antibodies using methods well known in the art, for example by proteolytic cleavage with enzymes such as papain (to produce Fab fragments) or pepsin (to produce F(ab′)₂ fragments).

The identification of these antigen binding region and/or epitopes recognized by mAbs of the present invention provides the information necessary to generate additional monoclonal antibodies with similar binding characteristics and therapeutic or diagnostic utility that parallel the embodiments of this application.

Recombinant murine or chimeric murine-human or human-human antibodies that bind an epitope included in the amino acid sequences residues 1-38 of SEQ ID NO: 1 can be provided according to the present invention using known techniques based on the teaching provided herein. See, e.g., Ausubel et al., eds. Current Protocols in Molecular Biology, Wiley Interscience, N.Y. (1987, 1992, 1993); and Sambrook et al. Molecular Cloning: A Laboratory Manual, Cold Spring Harbor Laboratory Press (1989), the entire contents of which are incorporated herein by reference.

The DNA encoding an antibody of the present invention can be genomic DNA or cDNA which encodes at least one of the heavy chain constant region (H_{c}), the heavy chain variable region (Hᵥ), the light chain variable region (Lᵥ) and the light chain constant regions (L_{c}). A convenient alternative to the use of chromosomal gene fragments as the source of DNA encoding the murine V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987), which references are hereby entirely incorporated herein by reference. The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

For example, a cDNA encoding a murine V region antigen-binding segment capable of binding to one or more T2DBMARKERS, for example, SEQ ID NO: 1, can be provided using known methods. Probes that bind a portion of a DNA sequence encoding the antibodies of the present invention can be used to isolate DNA from hybridomas expressing antibodies, fragments or regions, as presented herein, according to the present invention, by known methods.

Oligonucleotides representing a portion of the variable region are useful for screening for the presence of homologous genes and for the cloning of such genes encoding variable or constant regions of antibodies according to the invention. Such probes preferably bind to portions of sequences which encode light chain or heavy chain variable regions which bind an epitope of one or more T2DBMARKERS, especially an epitope of at least 5 amino acids of residues 1-38 of SEQ ID NO: 1. Such techniques for synthesizing such oligonucleotides are well known and disclosed by, for example, Wu, et al., Prog. Nucl. Acid. Res. Molec. Biol. 21:101-141 (1978), and Ausubel et al., eds. Current Protocols in Molecular Biology, Wiley Interscience (1987, 1993), the entire contents of which are herein incorporated by reference.

Because the genetic code is degenerate, more than one codon can be used to encode a particular amino acid (Watson, et al.). Using the genetic code, one or more different oligonucleotides can be identified, each of which would be capable of encoding the amino acid. The probability that a particular oligonucleotide will, in fact, constitute the actual XXX-encoding sequence can be estimated by considering abnormal base pairing relationships and the frequency with which a particular codon is actually used (to encode a particular amino acid) in eukaryotic or prokaryotic cells expressing an antibody of the invention or a fragment thereof. Such “codon usage rules” are disclosed by Lathe, et al., J. Molec. Biol. 183:1-12 (1985). Using the “codon usage rules” of Lathe, a single oligonucleotide, or a set of oligonucleotides, that contains a theoretical “most probable” nucleotide sequence capable of encoding preferred variable or constant region sequences is identified.

Although occasionally an amino acid sequence can be encoded by only a single oligonucleotide, frequently the amino acid sequence can be encoded by any of a set of similar oligonucleotides. Importantly, whereas all of the members of this set contain oligonucleotides which are capable of encoding the peptide fragment and, thus, potentially contain the same oligonucleotide sequence as the gene which encodes the peptide fragment, only one member of the set contains the nucleotide sequence that is identical to the nucleotide sequence of the gene. Because this member is present within the set, and is capable of hybridizing to DNA even in the presence of the other members of the set, it is possible to employ the unfractionated set of oligonucleotides in the same manner in which one would employ a single oligonucleotide to clone the gene that encodes the protein.

The oligonucleotide, or set of oligonucleotides, containing the theoretical “most probable” sequence capable of encoding an antibody of the present invention or fragment including a variable or constant region is used to identify the sequence of a complementary oligonucleotide or set of oligonucleotides which is capable of hybridizing to the “most probable” sequence, or set of sequences. An oligonucleotide containing such a complementary sequence can be employed as a probe to identify and isolate the variable or constant region gene (Sambrook et al., infra).

A suitable oligonucleotide, or set of oligonucleotides, which is capable of encoding a fragment of the variable or constant region (or which is complementary to such an oligonucleotide, or set of oligonucleotides) is identified (using the above-described procedure), synthesized, and hybridized by means well known in the art, against a DNA or, more preferably, a cDNA preparation derived from cells which are capable of expressing antibodies or variable or constant regions thereof. Single stranded oligonucleotide molecules complementary to the “most probable” variable or constant anti-T2DBMARKER region peptide coding sequences can be synthesized using procedures which are well known to those of ordinary skill in the art (Belagaje, et al., J. Biol. Chem. 254:5765-5780 (1979); Maniatis, et al., In: Molecular Mechanisms in the Control of Gene Expression, Nierlich, et al., Eds., Acad. Press, NY (1976); Wu, et al., Prog. Nucl. Acid Res. Molec. Biol. 21:101-141 (1978); Khorana, Science 203:614-625 (1979)). Additionally, DNA synthesis can be achieved through the use of automated synthesizers. Techniques of nucleic acid hybridization are disclosed by Sambrook et al. (infra), and by Haymes, et al. (In: Nucleic Acid Hybridization, A Practical Approach, IRL Press, Washington, D.C. (1985)), which references are herein incorporated by reference.

In an alternative way of cloning a polynucleotide encoding a variable or constant region, a library of expression vectors is prepared by cloning DNA or, more preferably, cDNA (from a cell capable of expressing an antibody or variable or constant region) into an expression vector. The library can then be screened for members capable of expressing a protein which competitively inhibits the binding of an antibody, and which has a nucleotide sequence that is capable of encoding polypeptides that have the same amino acid sequence as the antibodies of the present invention or fragments thereof. In this embodiment, DNA, or more preferably cDNA, is extracted and purified from a cell which is capable of expressing an antibody or fragment. The purified cDNA is fragmented (by shearing, endonuclease digestion, etc.) to produce a pool of DNA or cDNA fragments. DNA or cDNA fragments from this pool are then cloned into an expression vector in order to produce a genomic library of expression vectors whose members each contain a unique cloned DNA or cDNA fragment such as in a lambda phage library, expression in prokaryotic cell (e.g., bacteria) or eukaryotic cells, (e.g., mammalian, yeast, insect or, fungus). See, e.g., Ausubel, Harlow, Colligan; Nyyssonen et al. Bio/Technology 11:591-595 (Can 1993); Marks et al., Bio/Technology 11:1145-1149 (October 1993). Once a nucleic acid encoding such variable or constant regions is isolated, the nucleic acid can be appropriately expressed in a host cell, along with other constant or variable heavy or light chain encoding nucleic acid, in order to provide recombinant MAbs that bind one or more T2DBMARKERS with inhibitory activity. Such antibodies preferably include a murine or human variable region which contains a framework residue having complementarity determining residues which are responsible for antigen binding. Preferably, a variable light or heavy chain encoded by a nucleic acid as described above binds an epitope of at least 5 amino acids included within residues 1-38 of SEQ ID NO: 1.

Human genes which encode the constant (C) regions of the murine and chimeric antibodies, fragments and regions of the present invention can be derived from a human fetal liver library, by known methods. Human C regions genes can be derived from any human cell including those which express and produce human immunoglobulins. The human C_{H} region can be derived from any of the known classes or isotypes of human H chains, including γ, μ, α, δ or ε, and subtypes thereof, such as G1, G2, G3 and G4. Since the H chain isotype is responsible for the various effector functions of an antibody, the choice of C_{H} region will be guided by the desired effector functions, such as complement fixation, or activity in antibody-dependent cellular cytotoxicity (ADCC). Preferably, the C_{H} region is derived from gamma 1 (IgG1), gamma 3 (IgG3), gamma 4 (IgG4), or p (IgM). The human C_{L} region can be derived from either human L chain isotype, kappa or lambda.

Genes encoding human immunoglobulin C regions are obtained from human cells by standard cloning techniques (Sambrook, et al. (Molecular Cloning: A Laboratory Manual, 2nd Edition, Cold Spring Harbor Press, Cold Spring Harbor, N.Y. (1989) and Ausubel et al., eds. Current Protocols in Molecular Biology (1987-1993)). Human C region genes are readily available from known clones containing genes representing the two classes of L chains, the five classes of H chains and subclasses thereof. Chimeric antibody fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH₁ domain and hinge region of the H chain, followed by a translational stop codon to yield the truncated molecule.

Generally, the murine, human or murine and chimeric antibodies, fragments and regions of the present invention are produced by cloning DNA segments encoding the H and L chain antigen-binding regions of an antibody, and joining these DNA segments to DNA segments encoding C_{H} and C_{L} regions, respectively, to produce murine, human or chimeric immunoglobulin-encoding genes.

A fused chimeric gene can be created which comprises a first DNA segment that encodes at least the antigen-binding region of non-human origin, such as a functionally rearranged V region with joining (J) segment, linked to a second DNA segment encoding at least a part of a human C region. Therefore, cDNA encoding the antibody V and C regions, the method of producing the chimeric antibody according to the present invention involves several steps, involving isolation of messenger RNA (mRNA) from the cell line producing an antibody of the invention and from optional additional antibodies supplying heavy and light constant regions; cloning and cDNA production therefrom; preparation of a full length cDNA library from purified mRNA from which the appropriate V and/or C region gene segments of the L and H chain genes can be identified with appropriate probes, sequenced, and made compatible with a C or V gene segment from another antibody for a chimeric antibody; constructing complete H or L chain coding sequences by linkage of the cloned specific V region gene segments to cloned C region gene; expressing and producing L and H chains in selected hosts, including prokaryotic and eukaryotic cells to provide murine-murine, human-murine, human-human or human murine antibodies.

One common feature of all immunoglobulin H and L chain genes and their encoded mRNAs is the J region. H and L chain J regions have different sequences, but a high degree of sequence homology exists (greater than 80%) among each group, especially near the C region. This homology is exploited in this method and consensus sequences of H and L chain J regions can be used to design oligonucleotides for use as primers for introducing useful restriction sites into the J region for subsequent linkage of V region segments to human C region segments.

C region cDNA vectors prepared from human cells can be modified by site-directed mutagenesis to place a restriction site at the analogous position in the human sequence. For example, one can clone the complete human kappa chain C(C.sub.k) region and the complete human gamma-1 C region (C_{γ1}). In this case, the alternative method based upon genomic C region clones as the source for C region vectors would not allow these genes to be expressed in bacterial systems where enzymes needed to remove intervening sequences are absent. Cloned V region segments are excised and ligated to L or H chain C region vectors. Alternatively, the human C_{γ1} region can be modified by introducing a termination codon thereby generating a gene sequence which encodes the H chain portion of an Fab molecule. The coding sequences with linked V and C regions are then transferred into appropriate expression vehicles for expression in appropriate hosts, prokaryotic or eukaryotic.

Two coding DNA sequences are said to be “operably linked” if the linkage results in a continuously translatable sequence without alteration or interruption of the triplet reading frame. A DNA coding sequence is operably linked to a gene expression element if the linkage results in the proper function of that gene expression element to result in expression of the coding sequence.

Expression vehicles include plasmids or other vectors, which are used for carrying a functionally complete human C_{H} or C_{L} chain sequence having appropriate restriction sites engineered so that any V_{H} or V_{L} chain sequence with appropriate cohesive ends can be easily inserted therein. Human C_{H} or C_{L} chain sequence-containing vehicles thus serve as intermediates for the expression of any desired complete H or L chain in any appropriate host.

A chimeric antibody, such as a mouse-human or human-human, will typically be synthesized from genes driven by the chromosomal gene promoters native to the mouse H and L chain V regions used in the constructs; splicing usually occurs between the splice donor site in the mouse J region and the splice acceptor site preceding the human C region and also at the splice regions that occur within the human C region; polyadenylation and transcription termination occur at native chromosomal sites downstream of the human coding regions.

A nucleic acid sequence encoding at least one antibody or Ab fragment of the present invention may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Ausubel, infra, Sambrook, infra, entirely incorporated herein by reference, and are well known in the art.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression of antibodies or Ab fragments in recoverable amounts. The precise nature of the regulatory regions needed for gene expression may vary from organism to organism, as is well known in the analogous art. See, e.g., Sambrook, supra and Ausubel supra.

The present invention accordingly encompasses the expression of antibodies or Ab fragments, in either prokaryotic or eukaryotic cells, although eukaryotic expression is preferred. Preferred hosts are bacterial or eukaryotic hosts including bacteria, yeast, insects, fungi, bird and mammalian cells either in vivo, or in situ, or host cells of mammalian, insect, bird or yeast origin. It is preferable that the mammalian cell or tissue is of human, primate, hamster, rabbit, rodent, cow, pig, sheep, horse, goat, dog or cat origin, but any other mammalian cell may be used.

Further, by use of, for example, the yeast ubiquitin hydrolase system, in vivo synthesis of ubiquitin-transmembrane polypeptide fusion proteins can be achieved. The fusion proteins produced thereby may be processed in vivo or purified and processed in vitro, allowing synthesis of an antibody or Ab fragment of the present invention with a specified amino terminus sequence. Moreover, problems associated with retention of initiation codon-derived methionine residues in direct yeast (or bacterial) expression may be avoided. Sabin et al., Bio/Technol. 7(7): 705-709 (1989); Miller et al., Bio/Technol. 7(7):698-704 (1989).

Any of a series of yeast gene expression systems incorporating promoter and termination elements from the actively expressed genes coding for glycolytic enzymes produced in large quantities when yeast are grown in mediums rich in glucose can be utilized to obtain the antibodies or Ab fragments of the present invention. Known glycolytic genes can also provide very efficient transcriptional control signals. For example, the promoter and terminator signals of the phosphoglycerate kinase gene can be utilized.

Production of antibodies or Ab fragments or functional derivatives thereof in insects can be achieved, for example, by infecting the insect host with a baculovirus engineered to express a transmembrane polypeptide by methods known to those of skill. See Ausubel et al., eds. Current Protocols in Molecular Biology Wiley Interscience, 16.8-16.11 (1987, 1993).

In a preferred embodiment, the introduced nucleotide sequence will be incorporated into a plasmid or viral vector capable of autonomous replication in the recipient host. Any of a wide variety of vectors may be employed for this purpose. See, e.g., Ausubel et al., sections 1.5, 1.10, 7.1, 7.3, 8.1, 9.6, 9.7, 13.4, 16.2, 16.6, and 16.8-16.11. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Preferred prokaryotic vectors known in the art include plasmids such as those capable of replication in E. coli (such as, for example, pBR322, Co1E1, pSC101, pACYC 184, .pi.VX). Such plasmids are, for example, disclosed by Maniatis, T., et al. (Molecular Cloning, A Laboratory Manual, Second Edition, Cold Spring Harbor Press, Cold Spring Harbor, N.Y. (1989); Ausubel, infra. Bacillus plasmids include pC194, pC221, pT127, etc. Such plasmids are disclosed by Gryczan, T. (In: The Molecular Biology of the Bacilli, Academic Press, NY (1982), pp. 307-329). Suitable Streptomyces plasmids include pIJ101 (Kendall, K. J., et al., J. Bacteriol. 169:4177-4183 (1987)), and streptomyces bacteriophages such as .phi.C31 (Chater, K. F., et al., In: Sixth International Symposium on Actinomycetales Biology, Akademiai Kaido, Budapest, Hungary (1986), pp. 45-54). Pseudomonas plasmids are reviewed by John, J. F., et al. (Rev. Infect. Dis. 8:693-704 (1986)), and Izaki, K. (Jpn. J. Bacteriol. 33:729-742 (1978); and Ausubel et al., supra).

Alternatively, gene expression elements useful for the expression of cDNA encoding antibodies, antibody fragments, or peptides include, but are not limited to (a) viral transcription promoters and their enhancer elements, such as the SV40 early promoter (Okayama, et al., Mol. Cell. Biol. 3:280 (1983)), Rous sarcoma virus LTR (Gorman, et al., Proc. Natl. Acad. Sci., USA 79:6777 (1982)), and Moloney murine leukemia virus LTR (Grosschedl, et al., Cell 41:885 (1985)); (b) splice regions and polyadenylation sites such as those derived from the SV40 late region (Okayarea et al., infra); and (c) polyadenylation sites such as in SV40 (Okayama et al., infra).

Immunoglobulin cDNA genes can be expressed as described by Liu et al., infra, and Weidle et al., Gene 51:21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., Protein Engineering 1:499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences. For example, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. cDNA sequences encoding other proteins can also be combined with the above-recited expression elements to achieve expression of the proteins in mammalian cells.

Each fused gene can be assembled in, or inserted into, an expression vector. Recipient cells capable of expressing the chimeric immunoglobulin chain gene product are then transfected singly with the sequence encoding the antibody, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture. The fused genes encoding the antibodies or chimeric H and L chains, or portions thereof, can be assembled in separate expression vectors that are then used to co-transfect a recipient cell.

Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the fused genes in a bacterial system. The genes so produced and amplified in a bacterial host are subsequently used to co-transfect a eukaryotic cell, and allow selection of a co-transfected cell carrying the desired transfected genes.

Examples of selectable genes for use in a bacterial system are the gene that confers resistance to ampicillin and the gene that confers resistance to chloramphenicol. Preferred selectable genes for use in eukaryotic transfectants include the xanthine guanine phosphoribosyl transferase gene (designated gpt) and the phosphotransferase gens from Tn5 (designated neo). Selection of cells expressing gpt is based on the fact that the enzyme encoded by this gene utilizes xanthine as a substrate for purine nucleotide synthesis, whereas the analogous endogenous enzyme cannot. In a medium containing mycophenolic acid, which blocks the conversion of inosine monophosphate to xanthine monophosphate, and xanthine, only cells expressing the gpt gene can survive. The product of the neo blocks the inhibition of protein synthesis by the antibiotic G418 and other antibiotics of the neomycin class.

The two selection procedures can be used simultaneously or sequentially to select for the expression of immunoglobulin chain genes introduced on two different DNA vectors into a eukaryotic cell. It is not necessary to include different selectable markers for eukaryotic cells; an H and an L chain vector, each containing the same selectable marker can be co-transfected. After selection of the appropriately resistant cells, the majority of the clones will contain integrated copies of both H and L chain vectors and/or antibody fragments. Alternatively, the fused genes encoding the chimeric H and L chains can be assembled on the same expression vector.

For transfection of the expression vectors and production of the chimeric antibody, the preferred recipient cell line is a myeloma cell. Myeloma cells can synthesize, assemble and secrete immunoglobulins encoded by transfected immunoglobulin genes and possess the mechanism for glycosylation of the immunoglobulin. A particularly preferred recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

The expression vector carrying a chimeric antibody construct, antibody, or antibody fragment of the present invention can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment (Johnston et al., Science 240:1538 (1988)). A preferred way of introducing DNA into lymphoid cells is by electroporation (Potter et al., Proc. Natl. Acad. Sci. USA 81:7161 (1984); Yoshikawa, et al., Jpn. J. Cancer Res. 77:1122-1133). In this procedure, recipient cells are subjected to an electric pulse in the presence of the DNA to be incorporated. Typically, after transfection, cells are allowed to recover in complete medium for about 24 hours, and are then seeded in 96-well culture plates in the presence of the selective medium. G418 selection is performed using about 0.4 to 0.8 mg/ml G418. Mycophenolic acid selection utilizes about 6 μg/ml plus about 0.25 mg/ml xanthine. The electroporation technique is expected to yield transfection frequencies of about 10⁻⁵ to about 10⁻⁴ for Sp2/0 cells. In the protoplast fusion method, lysozyme is used to strip cell walls from catarrhal harboring the recombinant plasmid containing the chimeric antibody gene. The resulting spheroplasts can then be fused with myeloma cells with polyethylene glycol.

The immunoglobulin genes of the present invention can also be expressed in nonlymphoid mammalian cells or in other eukaryotic cells, such as yeast, or in prokaryotic cells, in particular bacteria. Yeast provides substantial advantages over bacteria for the production of immunoglobulin H and L chains. Yeasts carry out post-translational peptide modifications including glycosylation. A number of recombinant DNA strategies now exist which utilize strong promoter sequences and high copy number plasmids which can be used for production of the desired proteins in yeast. Yeast recognizes leader sequences of cloned mammalian gene products and secretes peptides bearing leader sequences (i.e., pre-peptides) (Hitzman, et al., 11th International Conference on Yeast, Genetics and Molecular Biology, Montpelier, France, Sep. 13-17, 1982).

Yeast gene expression systems can be routinely evaluated for the levels of production, secretion and the stability of antibody and assembled murine and chimeric antibodies, fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast (see Glover, ed., DNA Cloning, Vol. II, pp 45-66, IRL Press, 1985).

Bacterial strains can also be utilized as hosts for the production of antibody molecules or peptides described by this invention, E. coli K12 strains such as E. coli W3110 (ATCC 27325), and other enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of murine and chimeric antibodies, fragments and regions or antibody chains encoded by the cloned immunoglobulin cDNAs in bacteria (see Glover, ed., DNA Cloning, Vol. I, IRL Press, 1985, Ausubel, infra, Sambrook, infra, Colligan, infra).

Preferred hosts are mammalian cells, grown in vitro or in vivo. Mammalian cells provide post-translational modifications to immunoglobulin protein molecules including leader peptide removal, folding and assembly of H and L chains, glycosylation of the antibody molecules, and secretion of functional antibody protein. Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61).

Many vector systems are available for the expression of cloned antibodies, H and L chain genes, or antibody fragments in mammalian cells (see Glover, ed., DNA Cloning, Vol. II, pp 143-238, IRL Press, 1985). Different approaches can be followed to obtain complete H₂L₂ antibodies. As discussed above, it is possible to co-express H and L chains in the same cells to achieve intracellular association and linkage of H and L chains into complete tetrameric H₂L₂ antibodies and/or antibodies and/or antibody fragments of the invention. The co-expression can occur by using either the same or different plasmids in the same host. Genes for both H and L chains and/or antibodies and/or antibody fragments can be placed into the same plasmid, which can then be transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antibodies and/or H₂L₂ molecules and/or antibody fragments via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

In addition to monoclonal or chimeric antibodies, the present invention is also directed to an anti-idiotypic (anti-Id) antibody specific for the antibodies of the invention. An anti-Id antibody is an antibody which recognizes unique determinants generally associated with the antigen-binding region of another antibody. The antibody specific for one or more T2DBMARKERS, or SEQ ID NO: 1 is termed the idiotypic or Id antibody. The anti-Id can be prepared by immunizing an animal of the same species and genetic type (e.g. mouse strain) as the source of the Id antibody with the Id antibody or the antigen-binding region thereof. The immunized animal will recognize and respond to the idiotypic determinants of the immunizing antibody and produce an anti-Id antibody. The anti-Id antibody can also be used as an “immunogen” to induce an immune response in yet another animal, producing a so-called anti-anti-Id antibody. The anti-anti-Id can be epitopically identical to the original antibody which induced the anti-Id. Thus, by using antibodies to the idiotypic determinants of a mAb, it is possible to identify other clones expressing antibodies of identical specificity.

Accordingly, mAbs generated against one or more T2DBMARKERS according to the present invention can be used to induce anti-Id antibodies in suitable animals, such as BALB/c mice. Spleen cells from such immunized mice can be used to produce anti-Id hybridomas secreting anti-Id mAbs. Further, the anti-Id InAbs can be coupled to a carrier such as keyhole limpet hemocyanin (KLH) and used to immunize additional BALB/c mice. Sera from these mice will contain anti-anti-Id antibodies that have the binding properties of the original mAb specific for an epitope of a T2DBMARKER, or preferably, an epitope containing within amino acid residues 1-38 of SEQ ID NO: 1.

### Pharmaceutical Compositions and Methods of Treatment

The term “treating” in its various grammatical forms in relation to the present invention refers to preventing (i.e. chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessarily all symptoms) of a disease or attenuating the progression of a disease.

As used herein, the term “therapeutically effective amount” is intended to qualify the amount or amounts of T2DBMARKERS or other diabetes-modulating agents that will achieve a desired biological response. In the context of the present invention, the desired biological response can be partial or total inhibition, delay or prevention of the progression of type 2 Diabetes, pre-diabetic conditions, and complications associated with type 2 Diabetes or pre-diabetic conditions; inhibition, delay or prevention of the recurrence of type 2 Diabetes, pre-diabetic conditions, or complications associated with type 2 Diabetes or pre-diabetic conditions; or the prevention of the onset or development of type 2 Diabetes, pre-diabetic conditions, or complications associated with type 2 Diabetes or pre-diabetic conditions (chemoprevention) in a subject, for example a human.

The T2DBMARKERS, preferably included as part of a pharmaceutical composition, can be administered by any known administration method known to a person skilled in the art. Examples of routes of administration include but are not limited to oral, parenteral, intraperitoneal, intravenous, intraarterial, transdermal, topical, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form. The T2DBMARKERS or pharmaceutical compositions comprising the T2DBMARKERS can be administered in accordance with any dose and dosing schedule that achieves a dose effective to treat disease.

As examples, T2DBMARKERS or pharmaceutical compositions comprising T2DBMARKERS of the invention can be administered in such oral forms as tablets, capsules (each of which includes sustained release or timed release formulations), pills, powders, granules, elixirs, tinctures, suspensions, syrups, and emulsions. Likewise, the T2DBMARKERS or pharmaceutical compositions comprising T2DBMARKERS can be administered by intravenous (e.g., bolus or infusion), intraperitoneal, subcutaneous, intramuscular, or other routes using forms well known to those of ordinary skill in the pharmaceutical arts.

T2DBMARKERS and pharmaceutical compositions comprising T2DBMARKERS can also be administered in the form of a depot injection or implant preparation, which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

T2DBMARKERS or pharmaceutical compositions comprising T2DBMARKERS can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines. Liposomal preparations of diabetes-modulating agents may also be used in the methods of the invention.

T2DBMARKERS or pharmaceutical compositions comprising T2DBMARKERS can also be delivered by the use of monoclonal antibodies as individual carriers to which the compound molecules are coupled.

T2DBMARKERS or pharmaceutical compositions comprising T2DBMARKERS can also be prepared with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, T2DBMARKERS or pharmaceutical compositions comprising T2DBMARKERS can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The T2DBMARKERS or pharmaceutical compositions comprising T2DBMARKERS can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

Suitable pharmaceutically acceptable salts of the agents described herein and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

In addition, this invention also encompasses pharmaceutical compositions comprising any solid or liquid physical form of one or more of the T2DBMARKERS of the invention. For example, the T2DBMARKERS can be in a crystalline form, in amorphous form, and have any particle size. The T2DBMARKER particles may be micronized, or may be agglomerated, particulate granules, powders, oils, oily suspensions or any other form of solid or liquid physical form.

For oral administration, the pharmaceutical compositions can be liquid or solid. Suitable solid oral formulations include tablets, capsules, pills, granules, pellets, and the like. Suitable liquid oral formulations include solutions, suspensions, dispersions, emulsions, oils, and the like.

Any inert excipient that is commonly used as a carrier or diluent may be used in the formulations of the present invention, such as for example, a gum, a starch, a sugar, a cellulosic material, an acrylate, or mixtures thereof. The compositions may further comprise a disintegrating agent and a lubricant, and in addition may comprise one or more additives selected from a binder, a buffer, a protease inhibitor, a surfactant, a solubilizing agent, a plasticizer, an emulsifier, a stabilizing agent, a viscosity increasing agent, a sweetener, a film forming agent, or any combination thereof. Furthermore, the compositions of the present invention may be in the form of controlled release or immediate release formulations.

T2DBMARKERS can be administered as active ingredients in admixture with suitable pharmaceutical diluents, excipients or carriers (collectively referred to herein as “carrier” materials or “pharmaceutically acceptable carriers”) suitably selected with respect to the intended form of administration. As used herein, “pharmaceutically acceptable carrier or diluent” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions, or suspensions, including saline and buffered media. Examples of oils are those of petroleum, animal, vegetable, or synthetic origin, for example, peanut oil, soybean oil, mineral oil, olive oil, sunflower oil, and fish-liver oil. Solutions or suspensions can also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide.

Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCl, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

The preparation of pharmaceutical compositions that contain an active component is well understood in the art, for example, by mixing, granulating, or tablet-forming processes. The active therapeutic ingredient is often mixed with excipients that are pharmaceutically acceptable and compatible with the active ingredient. For oral administration, the active agents are mixed with additives customary for this purpose, such as vehicles, stabilizers, or inert diluents, and converted by customary methods into suitable forms for administration, such as tablets, coated tablets, hard or soft gelatin capsules, aqueous, alcoholic, or oily solutions and the like as detailed above.

For IV administration, Glucuronic acid, L-lactic acid, acetic acid, citric acid or any pharmaceutically acceptable acid/conjugate base with reasonable buffering capacity in the pH range acceptable for intravenous administration can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A particular pH range for intravenous formulation comprising an HDAC inhibitor, wherein the HDAC inhibitor has a hydroxamic acid moiety, can be about 9 to about 12.

Subcutaneous formulations can be prepared according to procedures well known in the art at a pH in the range between about 5 and about 12, which include suitable buffers and isotonicity agents. They can be formulated to deliver a daily dose of the active agent in one or more daily subcutaneous administrations. The choice of appropriate buffer and pH of a formulation, depending on solubility of one or more T2DBMARKERS to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12.

The compositions of the present invention can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

### EXAMPLES

### Example 1

### Biomarker Identification in the Cohen Rat Model of Type 2 Diabetes

The Cohen diabetic (CD) rat is a well-known and versatile animal model of Type 2 Diabetes, and is comprised of 2 rodent strains that manifest many of the common features of Type 2 Diabetes (T2D) in humans. The sensitive strain (CDs) develops Diabetes within 30 days when maintained on a high sucrose/copper-poor diet (HSD), whereas the resistant strain (CDr) retains normal blood glucose levels. When maintained indefinitely on regular rodent diet (RD), neither strain develop symptoms of T2D.

### Sample Preparation

Serum, urine, and tissue samples (including splenic tissue, pancreatic tissue, and liver tissue) were obtained from both CDr and CDs rats that were fed either RD or HSD for 30 days. The samples were flash-frozen and stored at −80° C.

Whole protein extracts were prepared for each of the 4 experimental conditions, utilizing 10 individual organs per group. Pancreatic tissues were processing using a mechanical shearing device (Polytron). To preserve protein integrity in processed samples, tissues were kept on dry ice until processing commenced and all buffers and equipment were pre-chilled. Samples were also kept on ice during the homogenization process.

T-Per buffer (Pierce) was pre-chilled on ice and two tablets of Complete Protease Inhibitor (Roche Applied Sciences) were added per 50 ml of buffer prior to use. Once protease inhibitors were added, any unused buffer was discarded. T-Per buffer was used at 20 ml per gram of tissue. For each group, pancreatic samples were weighed and the amount of lysis buffer required was calculated and added to each tissue sample in a 50 ml tube. Each sample was homogenized on ice for 10 seconds, followed by a 30 second rest period to allow the sample to cool. If gross debris was still apparent, the cycle was repeated until the homogenate was smooth. The homogenization probe was inserted into the samples approximately 1 cm from the bottom of the tube to minimize foaming. When homogenization was complete, the extract was centrifuged at 10,000×g for 15 minutes at 4° C.

Following centrifugation, the supernatant was harvested and a bicinchoninic acid (BCA) assay was performed to determine the total protein content. Table 2 provides the mean protein content of the samples corresponding to CDr rats fed either RD or HSD, and CDs rats fed either RD or HSD.

TABLE 2
Total Protein Content of Pancreatic Extracts from Cohen
Diabetic Rats

Mean Protein Content (μg/ml)

| Tissue | CDr-RD | CDr-HSD | CDs-RD | CDs-HSD |
|---|---|---|---|---|
| Pancreas | 7969.2 | 6061.9 | 6876.4 | 3387.8 |

Supernatants were dispensed into aliquots and stored at −80° C. Pelleted material was also kept and stored at −80° C.

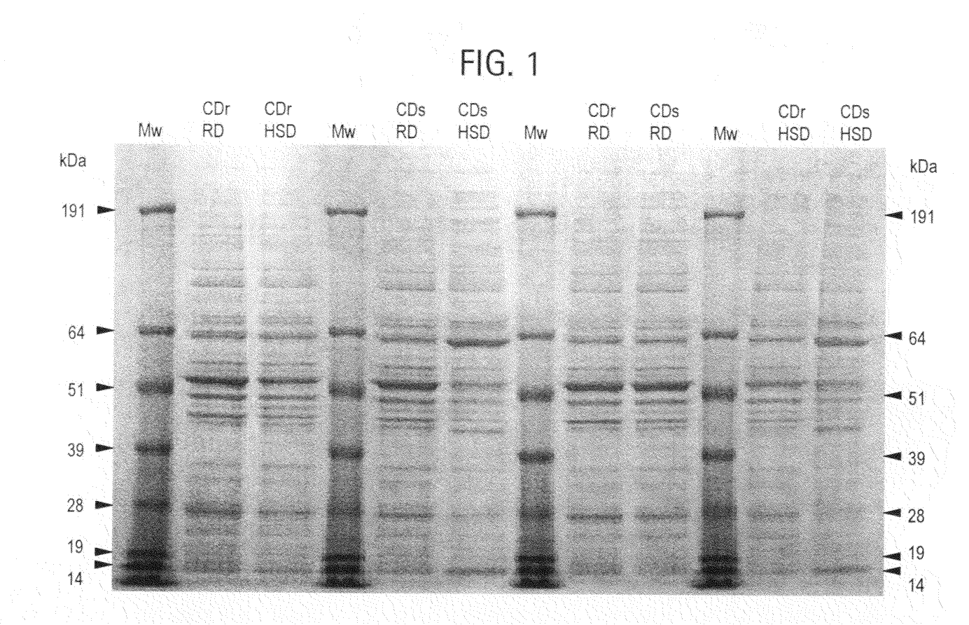

Protein expression profiling of the CDr and CDs phenotypes was conducted on the pancreatic extracts using one-dimensional SDS-PAGE. A sample of each extract containing 6 μg of total protein was prepared in sample buffer and loaded onto a 4-12% acrylamide gel. Following completion of the electrophoretic run, the gel was soaked with Coomassie stain for 1 hour and destained in distilled water overnight. The resulting protein expression profile allowed an empirical visual comparison of each extract (FIG. 1). These pancreatic extracts were then used for bi-directional immunological contrasting, disclosed herein.

Since albumin, immunoglobulin and other abundant proteins constitute about 95-97% of the total proteins in serum, the detection of less abundant proteins and peptides markers are masked if the whole serum were analyzed directly. Therefore, fractionation of serum samples was necessary to reduce masking of low abundance protein and to increase the number of peaks available for analysis.

To increase the detection of a larger number of peaks as well as to alleviate signal suppression effects on low abundance proteins from high abundant proteins such as albumin, immunoglobulin etc., the crude serum samples from CDr and CDs rats fed R^{D} or HSD were fractionated into six fractions. The fractionation was carried out using anion exchange bead based serum fractionate kit purchased from Ciphergen (Fremont, Calif.). In brief, the serum samples were diluted with a 9M urea denaturant solution; the diluted samples were then loaded onto a 96-well filter microplate pre-filled with an anion exchange sorbent. Using this process, samples were allowed to bind to the active surface of the beads, and after 30 minutes incubation at 4° C., the samples were eluted using stepwise pH gradient buffers. The process allowed the collection of 6 fractions including pH 9, pH 7, pH 5, pH 4, pH 3 and an organic eluent. After the fractionation, the serum samples were analyzed in the following formats on SELDI chips.

### SELDI (Surface Enhanced Laser Desorption Ionization)

SELDI Proteinchip® Technology (Ciphergen) is designed to perform mass spectrometric analysis of protein mixtures retained on chromatographic chip surfaces. The SELDI mass spectrometer produces spectra of complex protein mixtures based on the mass/charge ratio of the proteins in the mixture and their binding affinity to the chip surface. Differentially expressed proteins are determined from these protein profiles by comparing peak intensity. This technique utilizes aluminum-based supports, or chips, engineered with chemical modified surfaces (hydrophilic, hydrophobic, pre-activated, normal-phase, immobilized metal affinity, cationic or anionic), or biological (antibody, antigen binding fragments (e.g., scFv), DNA, enzyme, or receptor) bait surfaces. These varied chemical and biochemical surfaces allow differential capture of proteins based on the intrinsic properties of the proteins themselves. Tissue extractions or body fluids in volumes as small as 1 μl are directly applied to these surfaces, where proteins with affinities to the bait surface will bind. Following a series of washes to remove non-specifically or weakly bound proteins, the bound proteins are laser desorbed and ionized for MS analysis. Molecular weights of proteins ranging from small peptides to proteins (1000 Dalton to 200 kD) are measured. These mass spectral patterns are then used to differentiate one sample from another, and identify lead candidate markers for further analysis. Candidate marker have been identified by comparing the protein profiles of conditioned versus conditioned stem cell culture medium. Once candidate markers are identified, they are purified and sequenced.

The fractionated serum samples were applied to different chemically modified surface chips (cationic exchange, anionic exchange, metal-affinity binding, hydrophobic and normal phase) and profiled by SELDI, two-dimensional PAGE (2DE) and two-dimensional liquid chromatography (2D/LC).

### Two-dimensional Liquid Chromatography (2D/LC)

The ProteomeLab PF 2D Protein Fractionation System is a fully automated, two-dimensional fractionation system (in liquid phase) that resolves and collects proteins by isoelectric point (pI) in the first dimension and by hydrophobicity in the second dimension. The system visualizes the complex pattern with a two dimensional protein map that allows the direct comparison of protein profiling between different samples. Since all components are isolated and collected in liquid phase, it is ideal for downstream protein identification using mass spectrometry and/or protein extraction for antibody production.

The PF 2D system addresses many of the problems associated with traditional proteomics research, such as detection of low abundance proteins, run-to-run reproducibility, quantitation, detection of membrane or hydrophobic proteins, detection of basic proteins and detection of very low and very high molecular weight proteins. Since the dynamic range of proteins in serum spans over 10 orders of magnitude, and the relatively few abundant proteins make up over 95% of the total protein contents, this makes it very difficult to detect low abundant proteins that are candidate markers. In order to enrich and identify the less abundant proteins, the serum samples were partitioned using IgY-R7 rodent optimized partition column to separate the seven abundant proteins (Albumin, IgG, Transferrin, Fibrinogen, IgM, α1-Antitrypsin, Haptoglobin) from the less abundant ones.

The partitioned serum was applied to the PF-2D. The first dimensional chromatofocusing was performed on an HPCF column with a linear pH gradient generated using start buffer (pH 8.5) and eluent buffer (pH 4.0). The proteins were separated based on the pI. Fractions were collected and applied to a reverse-phase HPRP column for a second dimensional separation. The 2D map generated from each sample was then compared and differential peak patterns were identified. The fraction was subsequently selected and subjected to trypsin digestion. The digested samples were sequenced using LC/MS for protein identification.

### 2-D Gel Electrophoresis

Two-dimensional electrophoresis has the ability to resolve complex mixtures of thousands of proteins simultaneously in a single gel. In the first dimension, proteins are separated by pI, while in the second dimension, proteins are separated by MW. Applications of 2D gel electrophoresis include proteome analysis, cell differentiation, detection of disease markers, monitoring response to treatment etc.

The IgY partitioned serum samples were applied to immobilized pH gradient (IPG) strips with different pH gradients, pH 3-10, pH 3-6 and pH 5-8. After the first dimensional run, the IPG strip was laid on top of an 8-16% or 4-20% SDS-PAGE gradient gel for second dimensional separation.

### Results

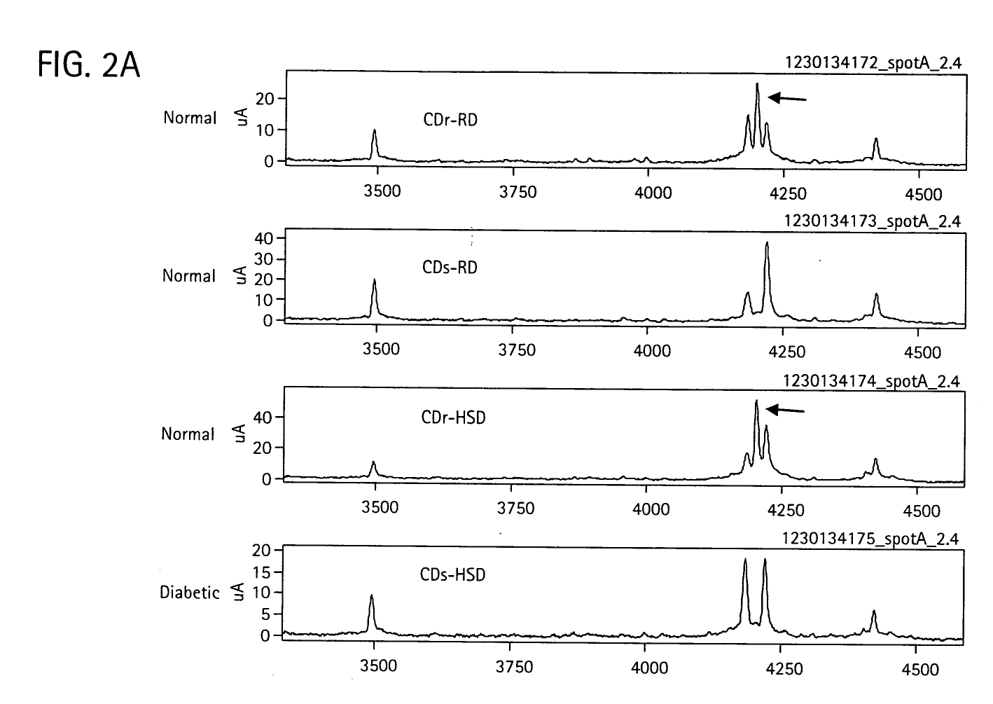

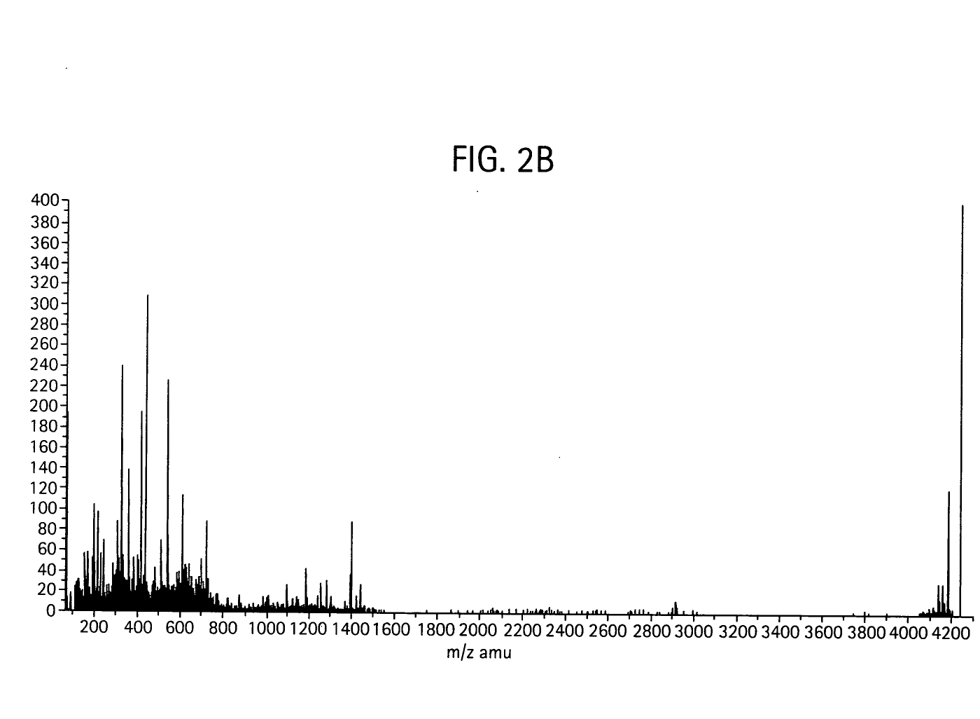

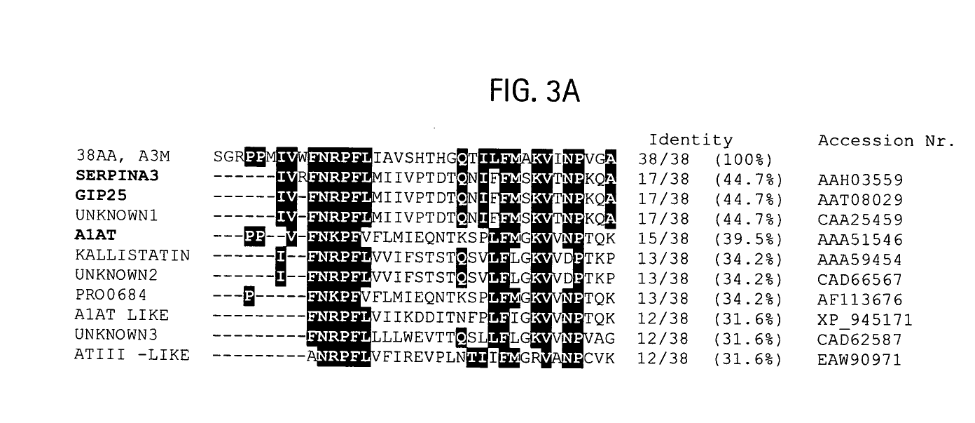

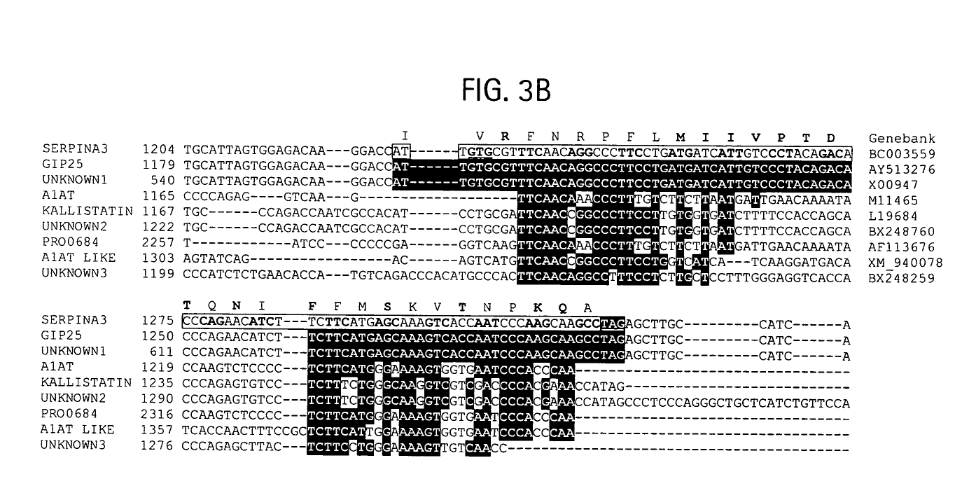

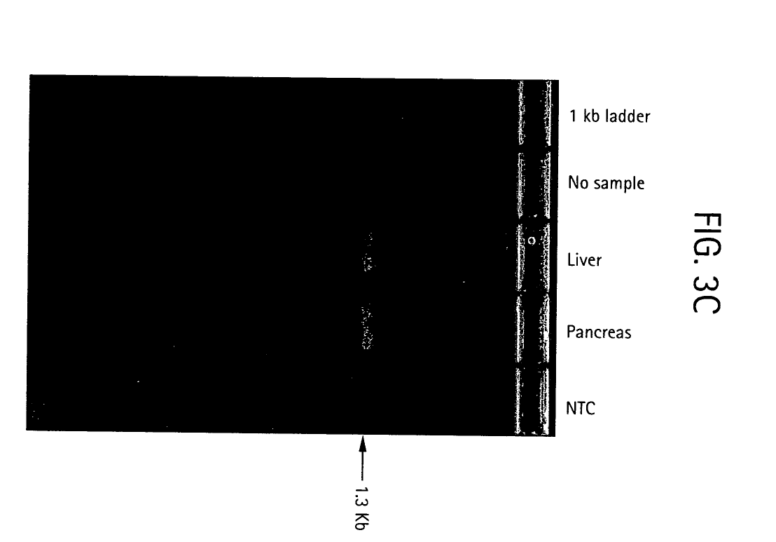

A peak protein of approximately 4200 daltons was present in the serum of CDr-RD and CDr-HSD, but not in the serum of CDs-RD or CDs-HSD, as shown in FIG. 2A. FIG. 2B is a MS/MS spectrum of the 4200 dalton fragment. This protein was sequenced and following extensive database searches, was found to be a novel protein. The peptide was designed “D3” and its sequence was found to be SGRPP MIVWF NRPFL IAVSH THGQT ILFMA KVINP VGA (SEQ ID NO: 1). The D3 peptide is a 38-mer peptide sequence that corresponds to the first biomarker discovered in the Cohen diabetic rat. Sequence alignment using the BLAST algorithm available from the National Center for Biotechnology Information (NCBI) was performed and the 38-amino acid fragment was found to have sequence identity with at least ten different amino acid sequences. Notably, BLAST alignment revealed that the 38-amino acid D3 peptide contains conserved motifs corresponding to: “FNRPFL” and “FMS/GKVT/VNP”. FIG. 3A shows the results of the BLAST alignment of amino acid sequences related to the D3 peptide fragment, and FIG. 3B shows the results of a BLAST alignment of nucleic acid sequences encoding the D3 peptide and the peptides identified by protein BLAST. Degenerate primers were designed to target the conserved motifs and comprise the following sequences: Forward primer (targeting regions containing the amino acid sequence “FNRPFL”: 5′-TTC AAC MRR CCY TTY ST-3′ (SEQ ID NO: 2) and Reverse primer (targeting regions containing the sequence “FMS/GKVT/VNP”): 5′-YVA CYT TKC YMA KRA AGA-3′ (SEQ ID NO: 3); wherein M=A or C; R=A or G; Y=C or T; S=C or G; K=G or T; and V=A, C, or G. These degenerate primers were used in reverse-transcription polymerase chain reactions (RT-PCR) to amplify human SERPINA 3 in liver and pancreas. A 1.3 Kb fragment was identified in human liver and pancreas, as shown in FIG. 3C.

Table 3 below represents additional indetified candidate markers indentified by SELDI analysis.

Array Type CM10 (Anion exchange)

| | | | | |
|---|---|---|---|---|
| | | CDs- | | |
| M/Z | CDr-RD | RD | CDr-HSD | CDs-HSD |

Sample Fractioned Serum F1

| | | | | |
|---|---|---|---|---|
| ~2156 | + | + | − | − |
| ~2270 | + | + | − | + |
| ~3875 | + | − | + | − |

Sample Fractioned Serum F3

| | | | | |
|---|---|---|---|---|
| ~3408 | − | + | − | + |
| ~3422 | + | − | + | − |
| ~3848 | − | + | − | + |
| ~3861 | + | − | + | − |

Sample Fractioned Serum F4

| | | | | |
|---|---|---|---|---|
| ~4202 | + | − | + | − |
| ~4423 | + | − | + | − |

Sample Fractioned Serum F5

| | | | | |
|---|---|---|---|---|
| ~5377 | ++ | ++ | ++ | + |
| ~5790 | +/− | +/− | − | + |
| ~8813 | +/− | +/− | +/− | + |

Sample Fractioned Serum F6

| | | | | |
|---|---|---|---|---|
| ~4200 | + | − | + | − |

Sample Whole Serum

| | | | | |
|---|---|---|---|---|
| ~6631 | − | + | − | − |
| ~7013 | − | − | + | + |
| ~7027 | + | + | − | − |
| ~7811 | − | + | − | − |

Array Type Q10

| | | | | |
|---|---|---|---|---|
| | | CDs- | | |
| M/Z | CDr-RD | RD | CDr-HSD | CDs-HSD |

Sample Fractioned Serum F1

| | | | | |
|---|---|---|---|---|
| ~2627 | + | − | + | − |
| ~2705 | + | − | + | − |
| ~4290 | + | + | ++ | + |
| ~5058 | − | − | + | − |
| ~5220 | + | ++ | + | + |
| ~5789 | − | − | + | − |
| ~8818 | + | +/− | ++ | ++ |

Sample Fractioned Serum F2

| | | | | |
|---|---|---|---|---|
| ~2359 | + | +/− | − | − |
| ~2587 | + | + | − | +/− |
| ~2879 | + | + | − | +/− |
| ~2298 | − | + | − | − |

Sample Fractioned Serum F4

| | | | | |
|---|---|---|---|---|
| ~4200 | + | − | + | − |
| ~2067 | − | − | + | + |
| ~2092 | − | − | + | + |
| ~2042 | − | − | + | + |
| ~8810 | − | − | + | + |
| ~8850 | + | + | − | − |

Sample Fractioned Serum F5

| | | | | |
|---|---|---|---|---|
| ~3977 | + | − | + | − |
| ~4200 | + | − | + | − |
| ~2102 | + | − | + | − |
| ~4030 | + | ++ | + | ++ |

Sample Fractioned Serum F6

| | | | | |
|---|---|---|---|---|
| ~4200 | + | − | + | − |
| ~17645 | + | − | + | − |

Sample Whole Serum

| | | | | |
|---|---|---|---|---|
| ~6632 | − | + | − | − |
| ~3419 | + | + | − | − |
| ~3435 | + | + | − | − |
| ~4074 | + | + | − | − |
| ~4090 | + | + | − | − |
| ~4200 | + | − | + | − |
| ~5152 | + | + | − | − |
| ~8915 | + | + | − | − |

Array Type H50

| | | | | |
|---|---|---|---|---|
| | | CDs- | | |
| M/Z | CDr-RD | RD | CDr-HSD | CDs-HSD |

Sample Fractioned Serum F2

| | | | | |
|---|---|---|---|---|
| ~5521 | − | + | − | − |

Sample Fractioned Serum F5

| | | | | |
|---|---|---|---|---|
| ~34224 | − | − | − | + |

Array Type IMAC

| | | | | |
|---|---|---|---|---|
| | | CDs- | | |
| M/Z | CDr-RD | RD | CDr-HSD | CDs-HSD |

Sample Whole Serum

| | | | | |
|---|---|---|---|---|
| ~2714 | + | + | − | + |
| ~4330 | − | + | + | + |

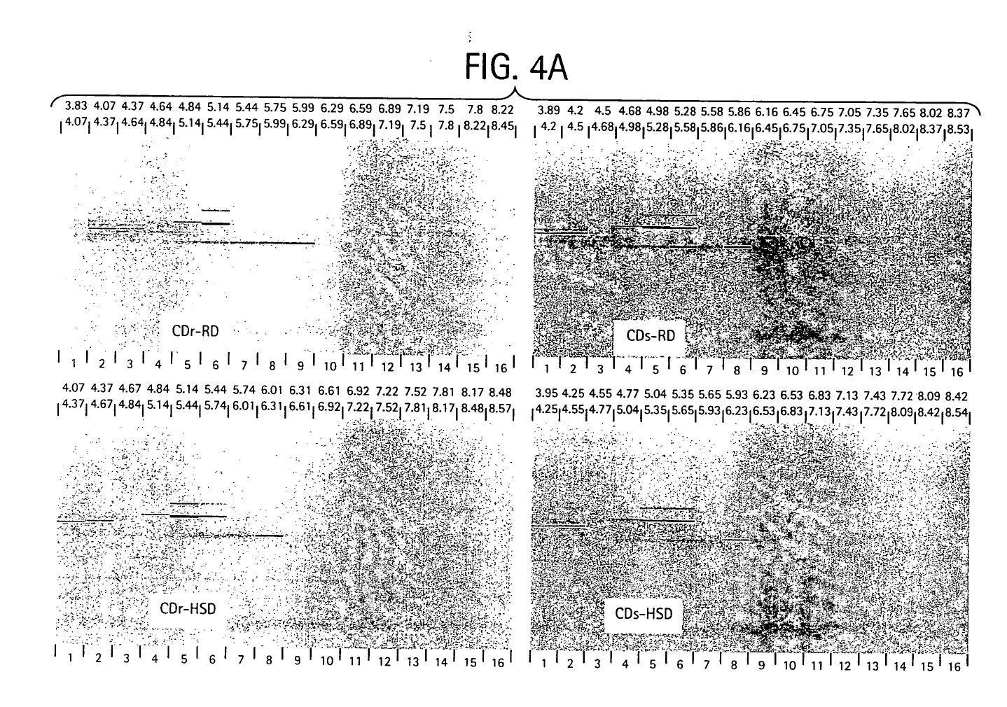

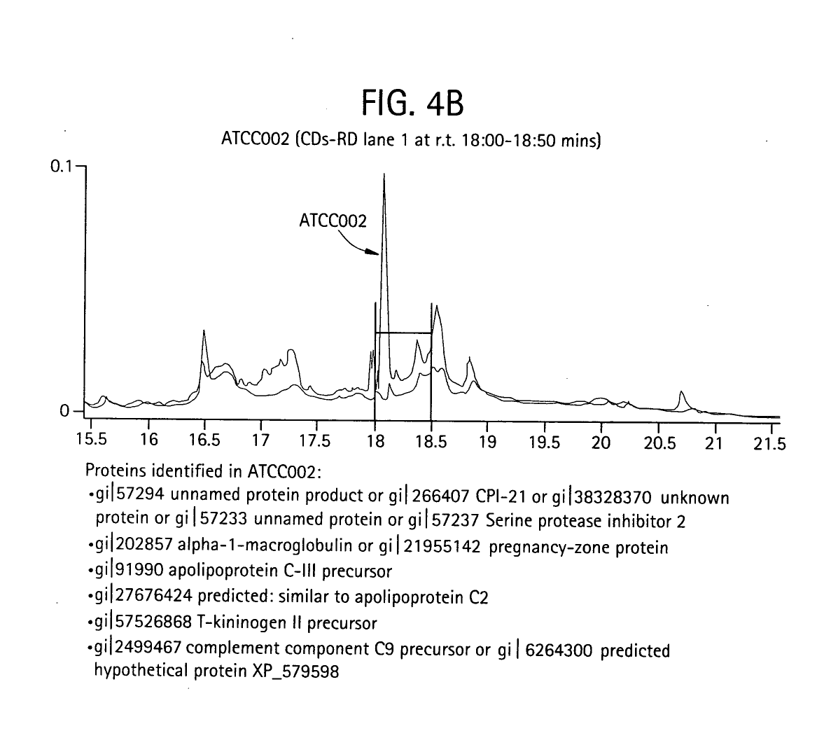

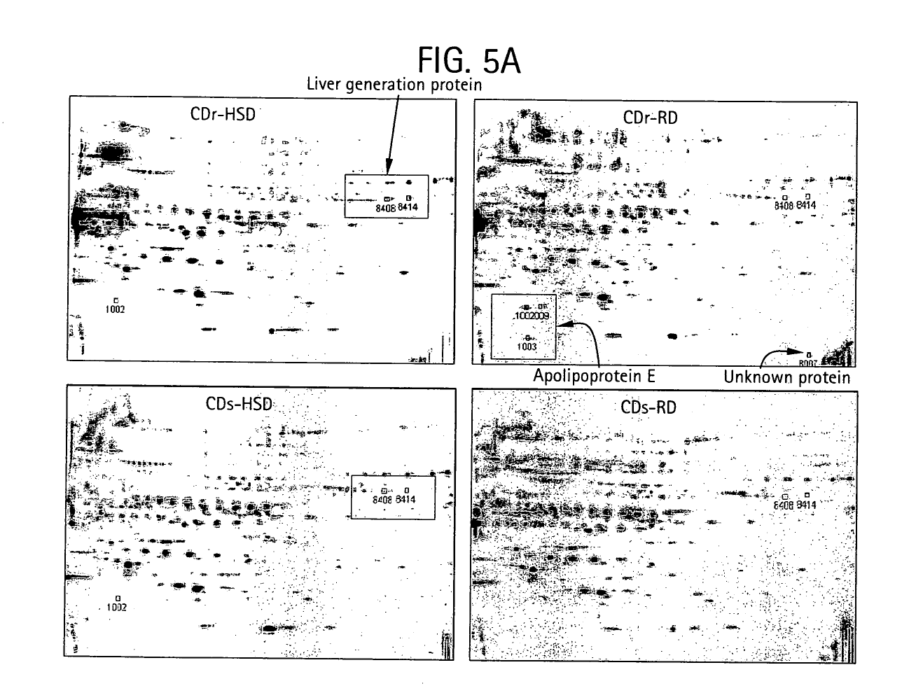

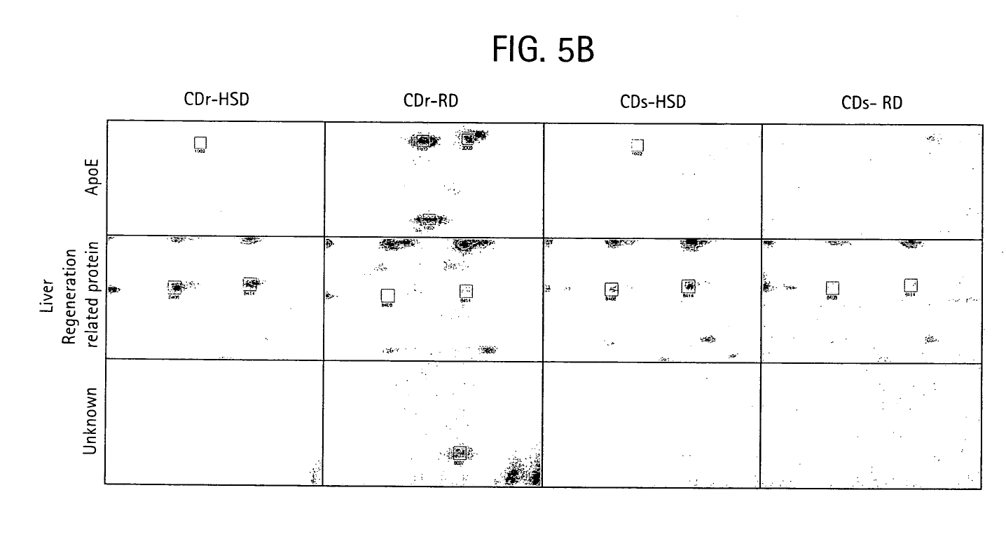

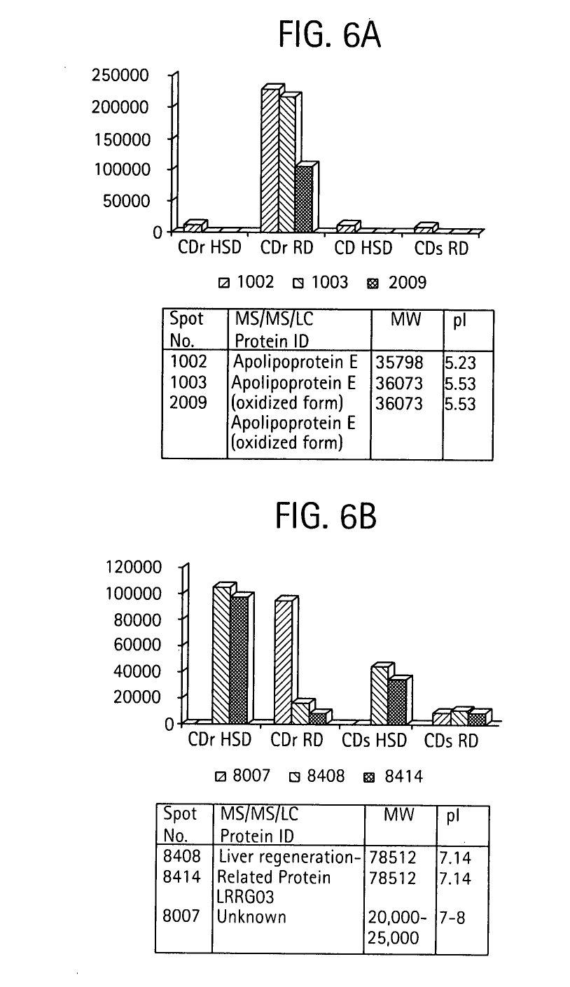

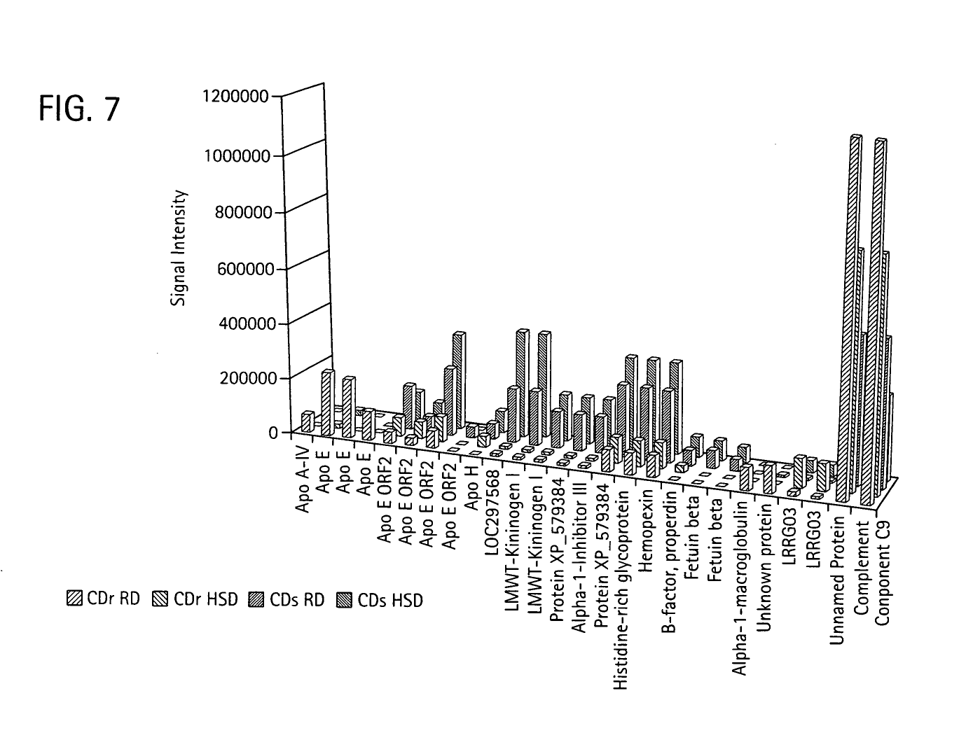

The differences among Cohen diabetic rats are shown in FIGS. 4A and 4B, which represent gels depicting biomarkers identified by LC/MS technology and a graph showing an elution profile obtained by differential two-dimensional reverse-phase HPLC or CDr-RD (red) versus CDs-RD (green) of a selected first dimension pI fraction (fraction 31). FIG. 5A represent 2DE gels of samples derived from each of the four Cohen diabetic rat models, while FIG. 5B is a magnified view of spots identified in FIG. 4A identified as apolipoprotein E, liver regeneration-related protein, and a previously unidentified protein. FIG. 6 is a graphical representation illustrating the differentially expressed proteins found in the four Cohen Diabetic rat models using 2DE technology. FIG. 7 is a histogram showing the differentially expressed Cohen Diabetic rat serum proteins identified by 2DE.

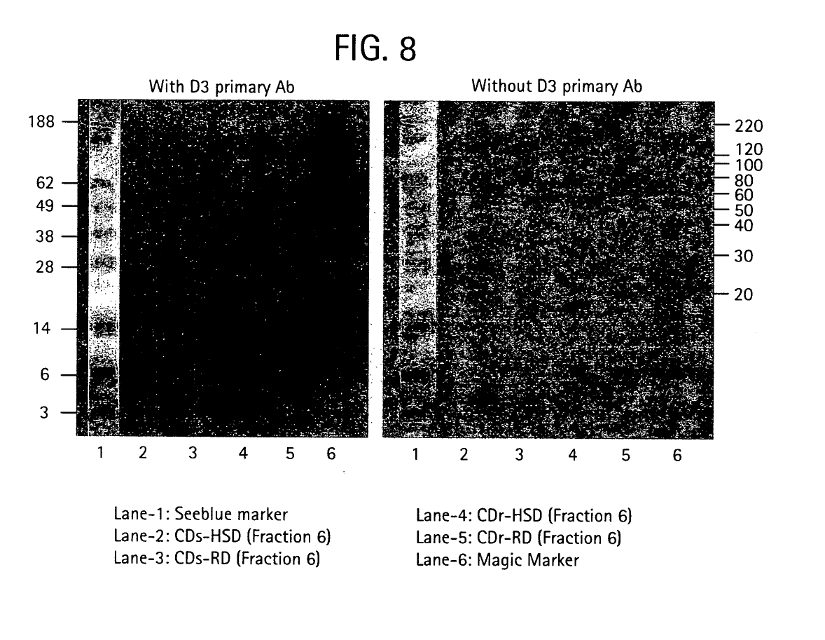

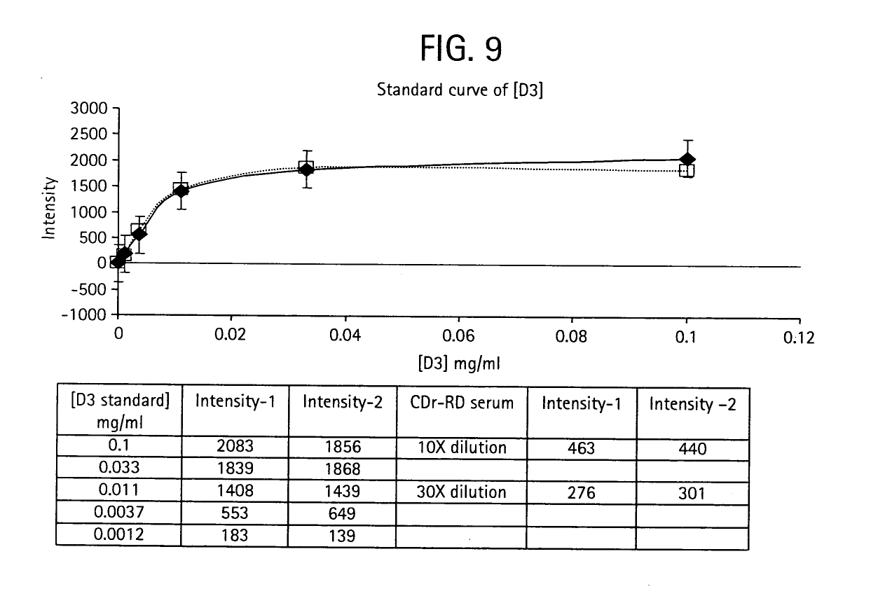

The D3 peptide was used for the production of hyper-immune serum in rabbits. FIG. 8 depicts Western blots showing the reactivity of the D3 hyper-immune serum with a 4 kD protein isolated from CDr-RD and CDr-HSD rat serum fraction 6. Fractionated CD rat serum samples were run on a 10% SDS-PAGE gel, then transferred to PVDF membranes. A higher molecular weight doublet (in the range of 49 and 62 kD) also reacted with the hyper-immune sera, indicated that a parent protein is expressed by all strains under treatment modalities RD or HSD, however a derivative of smaller size (˜4 kD) corresponding to the D3 fragment is differentially expressed only in the CDr strain. These results are consistent with the results obtained by SELDI profiling. The concentration of the D3 fragment in CDr rat serum was subsequently analyzed by SELDI. A series of synthetic D3 peptide standards (0.1, 0.033, 0.011, 0.0037, 0.0012 and 0 mg/ml) and 10× diluted CDr-serum were spotted in duplicate on Q10 protein chip arrays. The peak intensity was plotted against the concentration of D3 peptide standards. Based on the plot (FIG. 9), the linear range for concentration determination is from 0 to 0.01 mg/ml. Accordingly, the concentration of D3 in CDr-RD serum is around 0.04 mg/ml, based on the peak intensity of the CDr-RD serum sample.

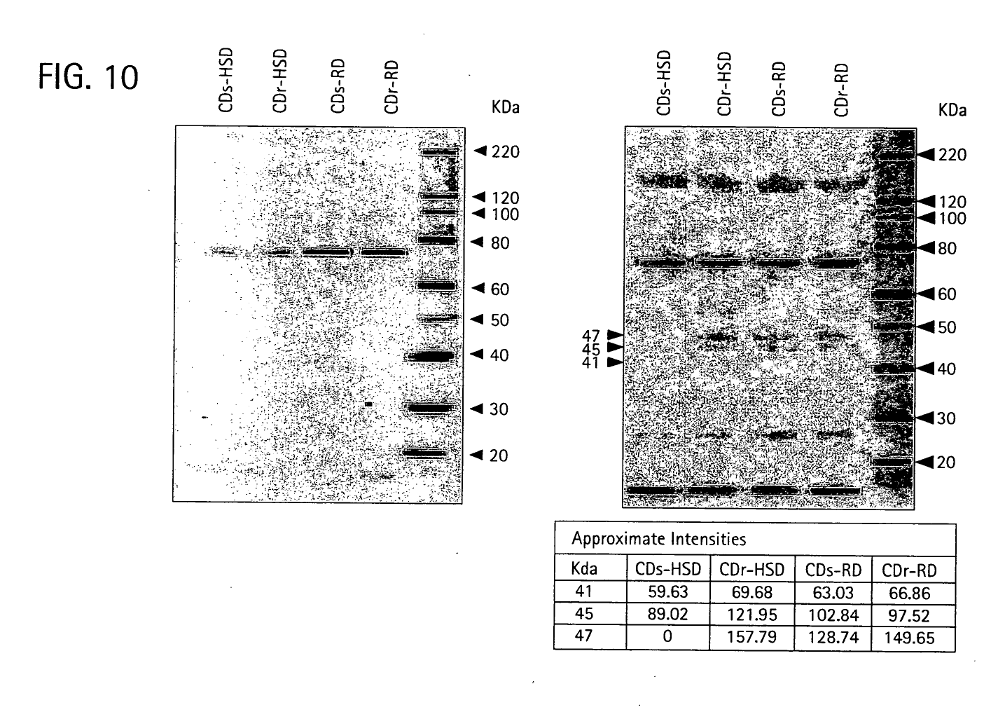

Analysis of Serpina expression by Western blot analysis was performed in Cohen rat liver extracts using anti D3 rabbit serum (1:200) and secondary goat anti-rabbit IgG conjugated to HRP (1:25,000 dilution). Controls containing liver extracts (10 μg) and secondary goat anti-rabbit IgG antibodies conjugated to HRP (1:25,000 dilution), but no primary antibody were also analyzed (FIG. 10). A cluster of proteins (41, 45 and 47 kD) were visualized following reaction of liver extracts with D3 hyper immune serum. The 41 and 45 kD proteins were expressed at approximately the same level while the 47 kD protein is not detected in the diabetic rat—i.e., CDs-HSD (diabetic).

Table 4 contains a summary of biomarker data obtained from CD rat serum samples.

TABLE 4
T2DBMARKER Data Summary

| | | | | | | Differential profiling in | | | | | |
|---|---|---|---|---|---|---|---|---|---|---|---|
| | | | | | | Cohen Diabetic Rats Serum | | | | | |
| | | | | MW | Calculated | | CDs- | CDr- | | Profiling | Human |
| No. | Protein | Gene | Gi | (KD) | pI | CDr-RD | RD | HSD | CDs-HSD | technology | Homologues |
| 1 | C-terminal fragment of a | Serpina | 34867677 | 4.2 | 12.01 | + | − | + | − | SELDI | Serpina 3 |
| | predicted protein, | 3M | | | | | | | | | |
| | similar to serine protease | | | | | | | | | | |
| | inhibitor 2.4 | | | | | | | | | | |
| 2 | unnamed protein product | Spin 2a | 57231 | 45 | 5.48 | + | − | − | − | PF-2D | |
| | or Spin2a protein | | 56789860 | 46 | 5.48 | | | | | | |
| 3 | Fetuin beta | Fetub | 17865327 | 42 | 6.71 | + | − | − | TBD | PF-2D result | Fetub_human |
| | or Fetub protein | | 47682636 | 44 | 7.47 | | | | | | |
| | | | | | | − | + | − | + | 2DE result | |
| 4 | Apolipoprotein C-III | Apoc 3 | 91990 | 11 | 4.65 | + | + | + | TBD | PF-2D | Apoc3_human |
| | precursor | | | | | | | | | | |
| 5 | Predicted protein, | Apoc 2 | 27676424 | 11 | 4.57 | + | + | + | − | PF-2D | Apoc2_human |
| | similar to | predicted | | | | | | | | | |
| | Apolipoprotein C2 | | | | | | | | | | |
| 6 | Aa2-066 | None | 33086518 | 61 | 4.39 | + | − | + | + | PF-2D | Alpha-2-HS- |
| | | | | | | | | | | | glycoprotein |
| | or alpha-2-HS- | Ahsg | 6978477 | 39 | 6.05 | | | | | | FetuA_Human |
| | glycoprotein | | | | | | | | | | |
| | or alpha-2-HS- | | 60552688 | 39 | 6.05 | | | | | | |
| | glycoprotein | | | | | | | | | | |
| 7 | T-kininogen II precursor | None | 57526868 | 49 | 5.94 | − | + | − | TBD | PF-2D | |
| 8 | alpha-1-macroglobulin | Pzp | 202857 | 168 | 6.46 | TBD | + | TBD | TBD | PF-2D result | PZP_human and |
| | or pregnancy-zone | Pzp | 21955142 | | | | | | | | A2MG_human |
| | protein | | | | | | | | | | |
| | | | | | | + | − | − | − | 2DE result | |
| 9 | Serine/cysteine | Serpinc1 | 56789738 | 53 | 6.18 | + | − | + | − | PF-2D | |
| | proteinase inhibitor, | | | | | | | | | | |
| | clade C, member 1 | | | | | | | | | | |
| | (predicted) | | | | | | | | | | |
| 10 | coagulation factor 2 | F2 | 12621076 | 72 | 6.28 | + | − | TBD | TBD | PF-2D | |
| 11 | inter-alpha-inhibitor H4 | ITIH4 | 9506819 | 104 | 6.08 | + | − | + | TBD | PF-2D | ITIH4_human |
| | heavy chain | | 59808074 | | | + | − | + | TBD | | |
| 12 | vitamin D binding | Gc | 203927 | 55 | 5.65 | + | − | TBD | TBD | PF-2D | VTDB_human |
| | protein prepeptide | | | | | | | | | | |
| 13 | LMW T-kininogen I | Map1 | 205085 | 49 | 6.29 | + | ++ | + | ++++ | PF-2D/2DE | |
| | precursor | | | | | | | | | | |
| | or kininogen | | 56270334 | | | | | | | | |
| | or major acute phase | | 68791 | | | | | | | | |
| | alpha-1 protein | | | | | | | | | | |
| | precursor | | | | | | | | | | |
| 14 | preapolipoprotein A-1 | ApoA1 | 55747 | 30 | 5.52 | + | + | + | − | PF-2D | ApoA1_human |
| | or apolipoprotein A-1 | | 59808388 | | | + | + | + | − | | |
| 15 | predicted protein, | Apoc2 | 109461385 | 11 | 4.57 | TBD | TBD | + | − | PF-2D | |
| | similar to apolipoprotein | | | | | | | | | | |
| | C-II precursor | | | | | | | | | | |
| 16 | thrombin | | 207304 | 28 | 9.38 | TBD | TBD | + | TBD | PF-2D | |
| | or prothrombin | | 56970 | 72 | | TBD | TBD | + | TBD | | THRB_human |
| | precursor | | | | | | | | | | |
| 17 | Apolipoprotein E | ApoE | 37805241 | 36 | 5.23 | + | − | − | − | 2DE | |
| | or Apolipoprotein E | | 55824759 | 36 | 5.53 | + | − | − | − | | |
| | or Apolipoprotein E | | 20301954 | 36 | | + | − | − | − | | |
| | or ORF2 | | 202959 | 38 | | + | − | − | − | | |
| | | | | | | + | ++ | + | ++ | | |
| 18 | Liver regeneration- | Tf | 33187764 | 78 | 7.14 | + | + | ++++ | ++ | 2DE | |
| | related protein LRRG03 | | | | | | | | | | |
| 19 | Apolipoprotein A-IV | Apoa4 | 60552712 | 44 | 5.12 | + | − | − | − | 2DE | |
| 20 | LOC297568 protein | | 71051724 | 79 | 5.45 | + | ++ | + | +++++ | 2DE | |
| | or Alpha-1-inhibitor 3 | | 112893 | 165 | | + | ++ | + | +++++ | | |
| | precursor | | | | | | | | | | |
| 21 | hypothetical protein | | 62718654 | 188 | 6.06 | + | ++ | + | +++ | 2DE | |
| | XP_579384 | | | | | | | | | | |
| 22 | Histidine-rich | Hrg | 11066005 | 59 | 8.12 | + | ++ | + | +++ | 2DE | |
| | glycoprotein | | | | | | | | | | |
| 23 | unnamed protein product | None | 55562 | 167 | 5.68 | +++ | ++ | ++ | + | 2DE | |
| | or predicted: | | 62647940 | 167 | | +++ | ++ | ++ | + | 2DE | |
| | hypothetical protein | | | | | | | | | | |
| | XP_579477 | | | | | | | | | | |
| 24 | Complement component | C9 | 2499467 | 63 | 5.51 | +++ | ++ | ++ | + | PF/2DE | |
| | C9 precursor | | | | | | | | | | |
| 25 | Apolipoprotein H | ApoH | 57528174 | 40 | 8.58 | − | + | + | + | 2DE | |
| 26 | B-factor, properdin | Cfb | 56268879 | 86 | 6.57 | − | + | + | + | 2DE | |
| 27 | Hemopexin | Hpx | 16758014 | 52 | 7.58 | + | ++ | + | +++ | 2DE | Hemo_human |

### Example 2

### Biomarker Identification in Human Sera

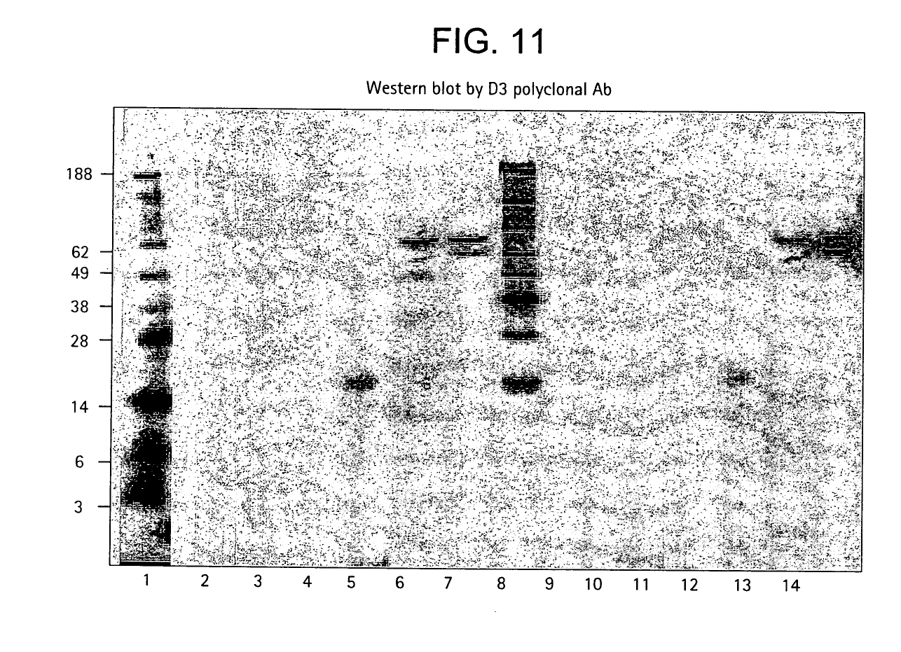

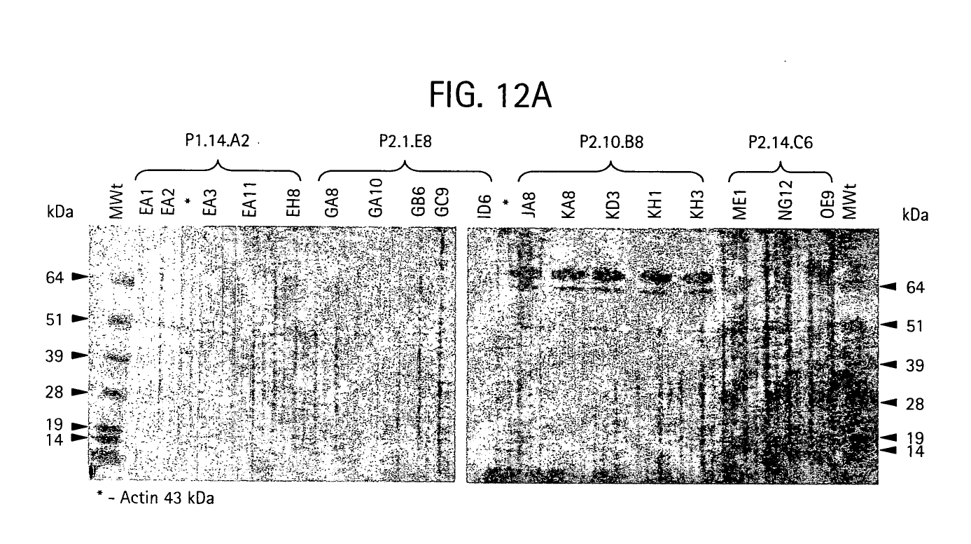

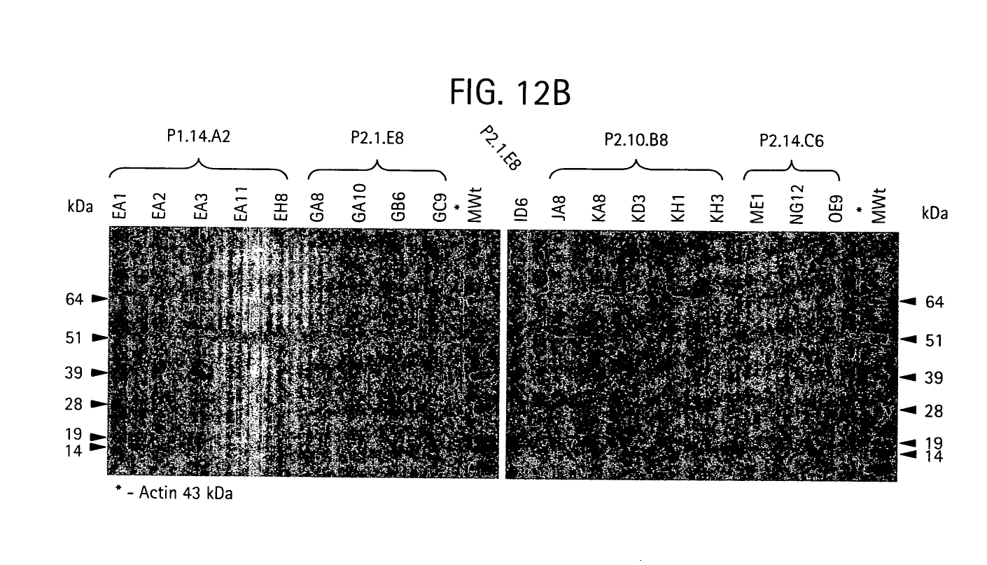

Analysis of human sera was performed using D3 hyper immune serum (rabbit; FIG. 11). The primary antibody used was rabbit polyclonal antibodies produced following immunization with D3 peptide. A protein with molecular weight of 20 kD (between the 14 kD and 28 kD markers) is expressed in human serum at a higher intensity in the normal individual as compared with Type 2 diabetic patient. A pair of proteins with MW of 60-80 kD appear to be present in both (normal and diabetic) samples. Interestingly, the intensity of the proteins in the doublet seemed to be inverted; an observation that was made using monoclonal antibodies derived from a subtractive immunization with CDr-HSD and CDs-HSD pancreas. FIGS. 12A and 12B show preparative gels containing 100 μg of CDr-HSD or CDs-HSD pancreatic extracts. The positive control was stained with 20 μg of anti-actin antibodies, and subclone lanes were stained with 600 μl of conditioned culture supernatant (described elsewhere in this disclosure).

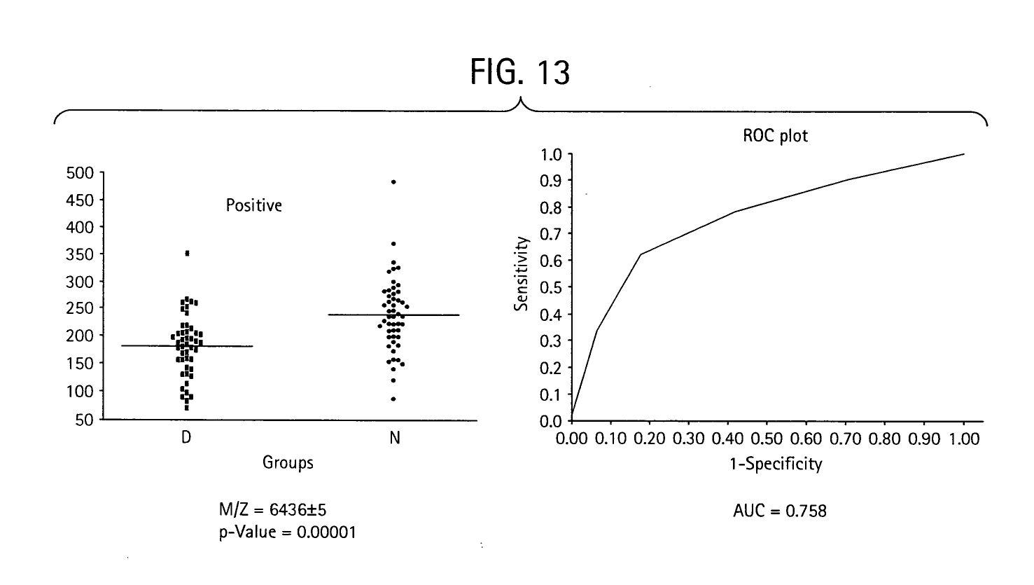

Human serum samples corresponding to samples taken from normal, diabetic and insulin-resistant subjects were obtained from three different sources and subjected to SELDI analysis: Dr. Itamar Raz, Dr. Wendell Cheatham, and Dr. Rachel Dankner. Dr. Raz's samples (hereinafter “Raz samples”) comprised 11 T2D human serum and plasma samples, and 9 normal human serum and plasma samples. The Cheatham samples comprised a total of 51 serum and urine samples, 12 of which were derived from Type 1 Diabetic individuals, 13 from T2D individuals, 10 insulin-resistant subjects, and 16 normal subjects. The Dankner samples comprised 23 T2D human serum samples and 25 normal human serum samples. SELDI analysis revealed the significant peaks from the Raz and Dankner samples, shown in Tables 5 and 6 below. FIG. 13 is an example of whole human serum profiled on anionic Q10 chips by SELDI.

TABLE 5
Selected significant peaks present in Raz samples

| | | | Fold Change |
|---|---|---|---|
| Sample No. | Peak (M/Z) | P-value | (T2D/N) |
| 1 | 12900 | 9.90E−07 | 3.24 |
| 2 | 134500 | 4.75E−06 | 0.55 |
| 3 | 44500 | 1.75E−05 | 2.21 |
| 4 | 4260 | 1.84E−05 | 0.4 |
| 5 | 4260 | 2.13E−05 | 0.49 |
| 6 | 56500 | 2.84E−05 | 0.55 |
| 7 | 6640 | 8.08E−05 | 2.14 |
| 8 | 12600 | 1.96E−04 | 2.64 |
| 9 | 2505 | 2.09E−04 | 1.71 |
| 10 | 29000 | 2.46E−04 | 0.63 |
| 11 | 3300 | 3.44E−04 | 0.65 |
| 12 | 14070 | 3.58E−04 | 0.69 |
| 13 | 11750 | 5.22E−04 | 2.81 |
| 14 | 6875 | 7.49E−04 | 2.2 |
| 15 | 13750 | 1.05E−03 | 0.66 |
| 16 | 9715 | 2.69E−03 | 1.89 |
| 17 | 9375 | 3.88E−03 | 1.61 |
| 18 | 6440 | 6.04E−03 | 2.1 |

TABLE 6
Selected significant peaks present in Dankner samples

| | | | Fold Change |
|---|---|---|---|
| Sample No. | Peak (M/Z) | P-value | (T2D/N) |
| 1 | 10075 | 4.81E−04 | 3.63 |
| 2 | 9310 | 1.87E−03 | 1.9 |
| 3 | 4160 | 3.68E−03 | 1.74 |
| 4 | 6450 | 1.59E−04 | 0.76 |
| 5 | 9310 | 8.25E−04 | 1.36 |
| 6 | 7770 | 8.25E−04 | 0.66 |
| 7 | 6430 | 1.32E−05 | 0.7 |
| 8 | 10650 | 2.25E−04 | 2.58 |

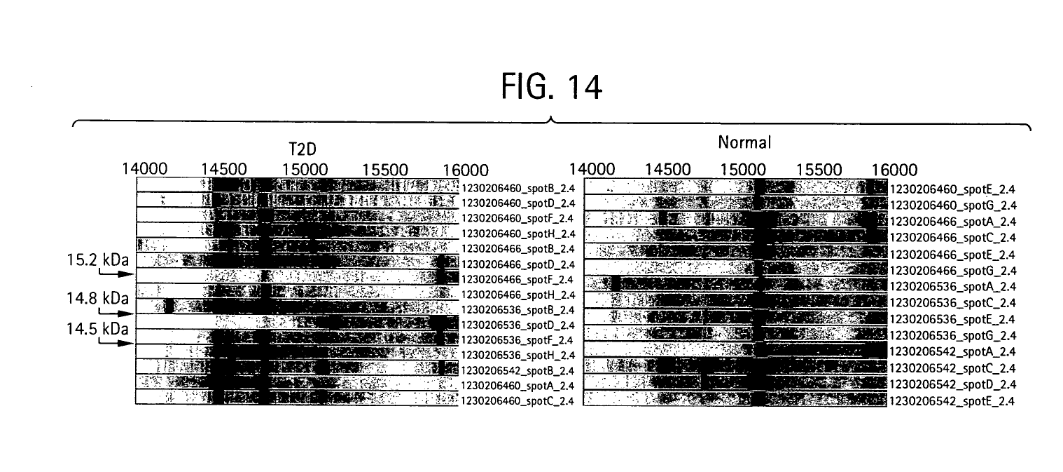

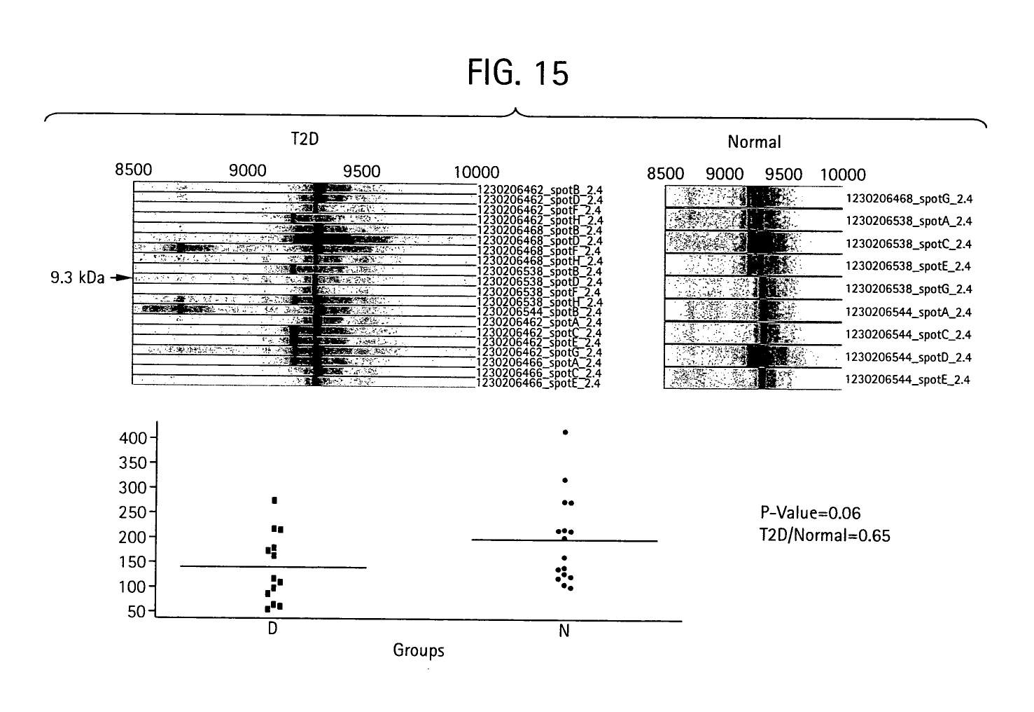

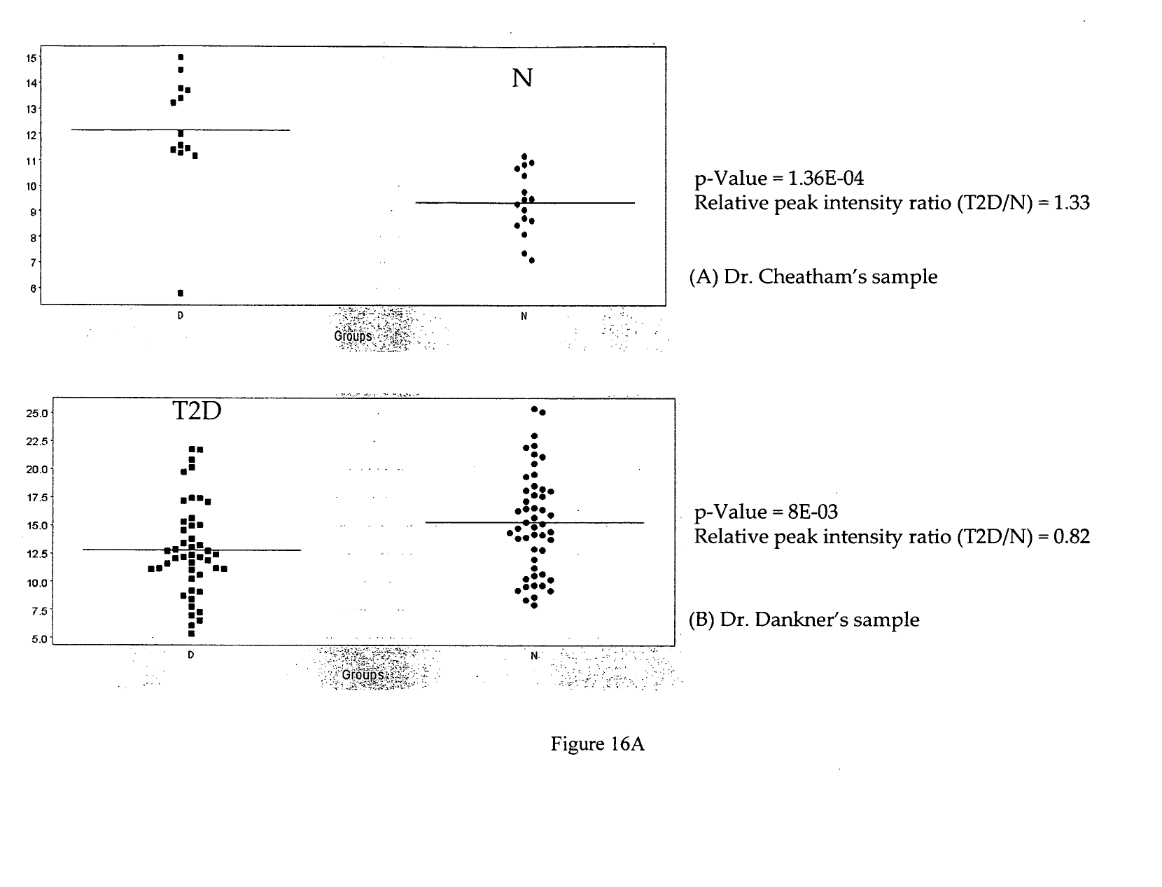

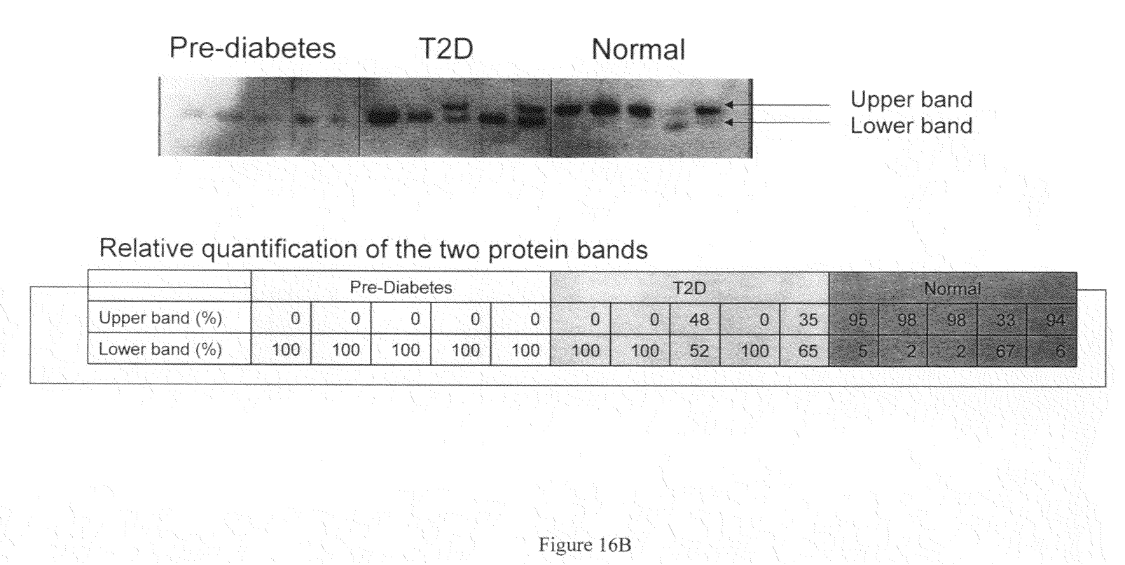

SELDI analysis revealed differentially expressed protein peaks identified in 13 T2D human samples and 16 normal human samples. FIG. 14 depicts a pseudogel view of SELDI analysis of Fraction 1 of the samples. Each lane represents a spectrum of an individual sample from M/Z 14.0 kD to 16.0 kD. The M/Z for the protein bands are approximately 15.2, 14.8, and 14.5 kD, respectively. FIG. 15 is another pseudogel view of SELDI analysis performed on 13 T2D and 16 normal fractionated serum samples (Fraction 3), profiled on a Q10 protein chip. Each lane represents the spectrum of an individual sample from M/Z 8.0 kD to 10.0 kD. The M/Z for the protein marker is approximately 9.3 kD. The graph below in FIG. 15 is a cluster view of a marker (M/Z 6430) that is downregulated in T2D samples. Levels of albumin were profiled using SELDI on the Cheatham samples and were compared to the Dankner samples, as shown in FIG. 16A.

Human serum samples from normal, pre-diabetic, and diabetic patients were also obtained from Dr. Cheatham. These samples were collected, fractionated, and resolved by SDS-PAGE. Immunoblotting was performed on the separated proteins using the rabbit anti-D3 polyclonal antibody disclosed herein. FIG. 166B shows the results of the immunoblot and the corresponding bands across pre-diabetic, T2D (diabetic), and normal subjects. The intensity of the protein bands of the immunoblot were quantified, demonstrating that, similar to the results obtained in FIG. 11, a doublet band having a molecular weight within the 60-80 kD range is expressed in human serum at a higher intensity in the normal individual as compared to patients diagnosed with Type 2 Diabetes.

### Example 3

### Bi-Directional Immunological Contrasting and Generation of Monoclonal Antibodies

From the pancreatic extract protein profiles obtained by SDS-PAGE, obvious differences in the banding patterns were noted between CDr-HSD and CDs-HSD samples (FIG. 1). Bi-directional immunological contrast was performed between these two samples. This technique involves injecting two pancreatic extracts from the Cohen diabetic rats to be contrasted separately into the footpads of an experimental animal (e.g. a Balb/c mouse). Following uptake and processing of the antigen at the site of injection by antigen presenting cells (APCs), the activated APCs migrate to the local lymph nodes (popliteal) to initiate an immune response. As these lymph nodes are located in each leg, they are anatomically separated from each other, which prevents mixing of antigen-specific lymphocytes at this point. Later in the immune response, these activated lymphocytes migrate from the local lymph nodes to the spleen where they become mixed, and from where they may circulate systemically.

Two weeks after footpad injection, the animals were boosted by injecting each footpad with the same antigen as before. This boost recalls antigen specific lymphocytes back to the site of injection, again subsequently draining to the popliteal lymph nodes. This technique uses the natural proliferation and cell migration processes as a filtering mechanism to separate and enrich specific lymphocytes in each lymph node, where they are anatomically segregated to minimize mixing of cells that are specific for antigen(s) expressed in only one of the extracts. Three days after boosting, the popliteal lymph nodes were removed and separated into pools derived from each side of the animals. When boosting, it is imperative not to switch the antigenic material, as this will cause specific lymphocytes to migrate to both sets of popliteal lymph nodes and the anatomical segregation of specific cells, and hence the advantage of the technique, will be lost.

Fifteen female Balb/c mice ages 6-8 weeks were ordered from Harlan. Each animal was injected with 25 μg of CDr-HSD pancreatic extract into the left hind footpad, and 25 μg of CDs-HSD pancreatic extract into the right hind footpad. Antigens were prepared in 20% Ribi adjuvant in a final volume of 50 μl as follows:

TABLE 7

| | Right footpad | Left footpad |
|---|---|---|
| 375 mg of CDs-HSD | 110 μl | — |
| 375 mg of CDr-HSD | — | 62 μl |
| PBS | 490 μl | 538 μl |
| Ribi adjuvant | 150 μl | 150 μl |

Ribi adjuvant was warmed to 37° C. and reconstituted with 1 ml of sterile PBS. The bottle was vortexed for at least 1 minute to fully reconstitute the material. The correct volume of Ribi adjuvant was then added to the antigen preparation, and the mixture was again vortexed for 1 minute. Any unused formulated material was discarded, and any unused Ribi adjuvant was stored at 4° C. and used to formulate booster injections. Animals were primed on day 1 and boosted on day 14. Animals were euthanized on day 17, when popliteal lymph nodes were excised post mortem and returned to the lab for processing.

### Generation of Hybridomas

Hybridoma cell lines were created essentially as described by Kohler and Milstein (1975). Lymphocytes derived from immunized animals were fused with a murine myeloma cell line (Sp2/0) by incubation with polyethylene glycol (PEG). Following fusion, cells were maintained in selective medium containing hypoxanthine, aminopterin and thymidine (HAT medium) that facilitates only the outgrowth of chimeric fused cells.

On the day before the fusion, the fusion partner (Sp2/0x Ag14 cells in dividing stage with viability above 95%) was split at 1×10⁵ viable cells/ml, 24 hours before the fusion. On the day of the fusion, the mice were sacrificed and the lymph nodes were excised and placed in a Petri dish containing pre-warmed room temperature DMEM supplemented with 10% fetal bovine serum (FBS). Using sterile microscope slides, the lymph nodes were placed between the 2 frosty sides of the slides and crushed into a single cell suspension. The cell suspension was then transferred to a 15 ml tube and centrifuged for 1 minute at 1000 rpm. The supernatant was removed by aspiration, and the cell pellet gently resuspended in 12 ml of serum-free DMEM, after which they were subjected to another round of centrifugation for 10 minutes at 1000 rpm. The process was repeated twice more to ensure that the serum was completely removed. After washing, the cells were resuspended in 5 ml of serum-free DMEM and counted under the microscope.

The fusion partner was collected by spinning in a centrifuge for 10 minutes at 1000 rpm. The cells were washed three times in serum-free DMEM, and finally resuspended in serum-free DMEM and counted. The number of fusion partner cells were calculated based on the number of lymph node cells. For every myeloma cell (fusion partner), 2 lymph nodes cells is needed (ratio 1:2 of myeloma to lymph node cells; e.g. for 10×10⁶ lymph node cells, 5×10⁶ fusion partner cells are needed). The appropriate number of myeloma cells to the LN cells were added and the total volume of cells was adjusted to 25 ml using serum free DMEM, and 25 ml of 3% dextran was then added to the cells. The mixture was spun for 10 minutes at 1000 rpm, and the supernatant aspirated as much as possible from the cell pellet. Once the lid was placed onto the tube containing the cells, the bottom of the tube was gently tapped the bottom of the tube to resuspend the cells and 1 ml of pre-warmed 50% (v/v) PEG was added to the tube. The agglutinated cells were allowed to sit for 1 minute, after which 20 ml of serum free DMEM, followed by 25 ml of 20% FBS, DMEM with 25 mM Hepes was added. The tube was inverted once to mix and then centrifuged for 10 minutes at 1000 rpm. The media was aspirated and the cells were gently resuspended by tapping. HAT selection media was added such that the cell suspension was either at 0.125×10⁶ cells/ml or 0.0625×10⁶ cells/ml. One hundred 111 of cells per well were added to a 96-well flat bottom plate and incubated at 37° C. with CO₂ at 8.5%. After 2 days, the cells were fed with 100 μl of fresh HAT selection media. Cells were checked for colony growth after 7 days.

### Hybridoma Screening

Once visible colonies were observed in the 96 well plates, 100 μl of conditioned supernatant was harvested from each colony for screening by ELISA. Supernatants were screened for the presence of detectable levels of antigen-specific IgG against both CDr-HSD and CDs-HSD extracts. Only colonies exhibiting a positive ELISA reaction against one of the two extracts with at least a 2-fold difference were selected for expansion and further characterization.

Pancreas extract at a concentration of 25 μg/ml to be tested was diluted in carbonate bicarbonate buffer (1 capsule of carbonate-bicarbonate was dissolved in 100 ml of deionized water). Two extra wells for the positive control and two extra wells for the negative control of a 96-well plate were reserved. The plate was then covered using adhesive film and incubated at 4° C. overnight.

The plate was washed once with 200 μl of PBS/Tween. The well content was removed by flicking the plate into a sink, and then gently tapping the plate against absorbent paper to remove remaining liquid. Approximately 200 μl of washing buffer (PBS/Tween) was added and subsequently discarded as previously described. The entire plate was then blocked for 1 hour at 37° C. in 200 μl of 5% powdered milk/PBS/Tween. The plate was then washed 3 times using PBS/Tween as previously described.

The fusion culture supernatant was diluted 1:1 in 0.5% milk/PBS/Tween and each sample added to the wells (50 μl; final volume is 100 μl per well) with 50 μl of anti-actin Ab (Sigma) at 20 μg/ml to well containing 50 μl of buffer. Fifty μl of buffer was added to the negative control well. The plate was covered and incubated overnight at 4° C. The plate was washed 3 times using PBS/Tween as previously described, and anti-HRP anti-mouse IgG in 0.5% milk/PBS/Tween at 1:20000 (100 μl) was added to each well. The plate was covered and incubated at 37° C. for two hours.

After incubation with secondary antibody, the plates were washed 4 to 5 times as previously described. On the last wash, the washing buffer was left on the plate for a couple of minutes before discarding it. One hundred μl of pre-warmed room temperature TMB (VWR; stored in the dark) was added to each well while minimizing the introduction of bubbles, until the color developed (20-30 minutes). The reaction was stopped by adding 50 μl of 2M sulfuric acid. The plate was read using a spectrophotometer at 450 nm.

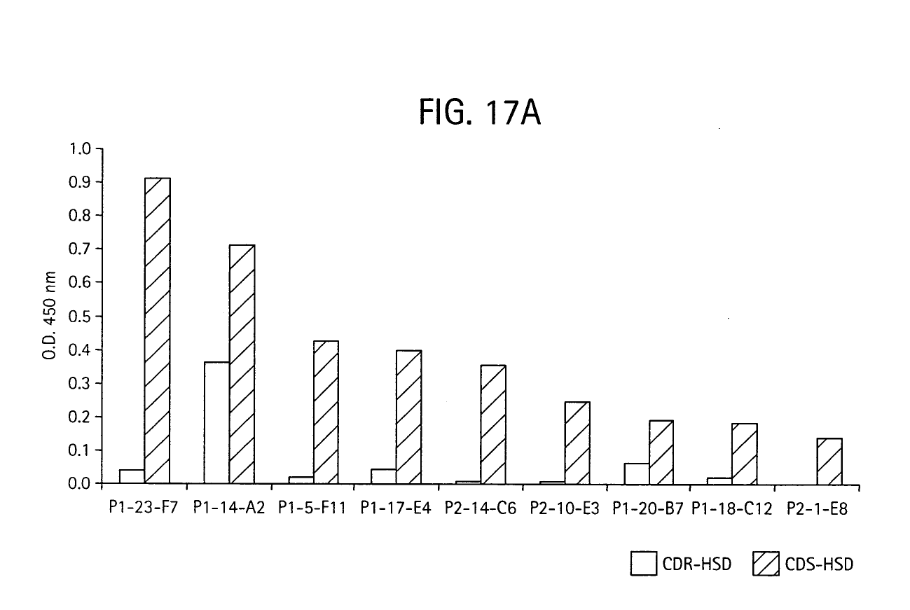

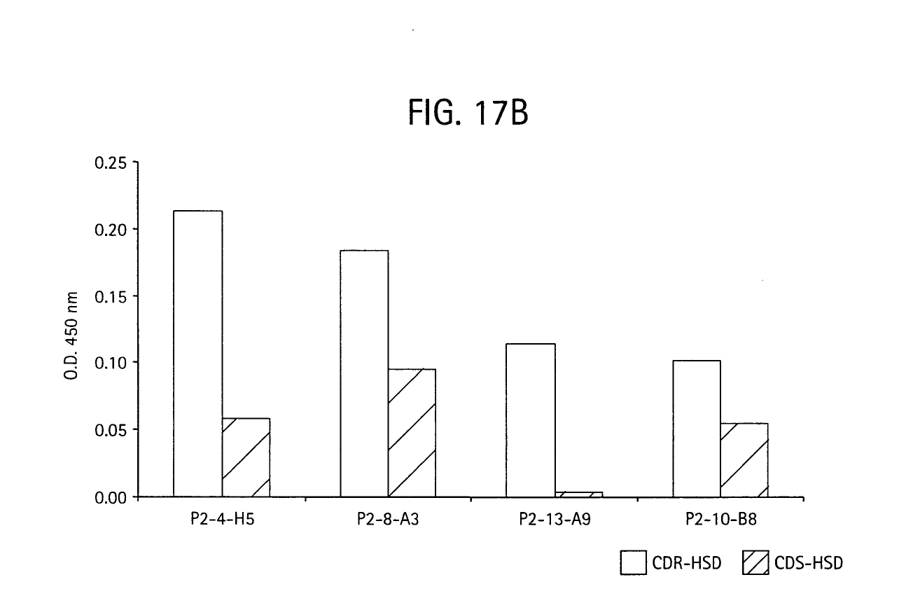

Thirteen clones produced monoclonal antibodies (mAbs) that met the experimental criteria outlined above, 9 against CDs-HSD and 4 against CDr-HSD. The ELISA data for these colonies is summarized in Table 5 and graphically represented in FIGS. 17A and 17B. Table 8 shows ELISA screening data for monospecific CDr-HSD and CDs-HSD hybridomas. Absolute absorbance values, and fold difference at OD 450 nm is shown for each colony. To verify primary screening data, some clones were retested during expansion to confirm the experimental observations from the initial screen.

TABLE 8

| | Primary Screen | | | Confirmatory Screen | | |
|---|---|---|---|---|---|---|
| Clone ID | | | Fold | | | Fold |
| Accession No. | CDR-HSD | CDS-HSD | Difference | CDR-HSD | CDS-HSD | Difference |
| P1-5-F11 | 0.021 | 0.426 | 20.29 | 0.013 | 0.192 | 14.77 |
| (Accession | | | | | | |
| No.) | | | | | | |
| P1-14-A2 | 0.363 | 0.714 | 1.97 | NT | NT | — |
| (Accession | | | | | | |
| No.) | | | | | | |
| P1-17-E4 | 0.042 | 0.398 | 9.48 | NT | NT | — |
| (Accession | | | | | | |
| No.) | | | | | | |
| P1-18-C12 | 0.021 | 0.183 | 8.71 | NT | NT | — |
| (Accession | | | | | | |
| No.) | | | | | | |
| P1-20-B7 | 0.065 | 0.192 | 2.95 | 0.025 | 0.110 | 4.40 |
| (Accession | | | | | | |
| No.) | | | | | | |
| P1-23-F7 | 0.039 | 0.912 | 23.38 | 0.046 | 0.547 | 11.89 |
| (Accession | | | | | | |
| No.) | | | | | | |
| P2-1-E8 | 0.001 | 0.139 | 139.00 | 0.019 | 0.252 | 13.26 |
| (Accession | | | | | | |
| No.) | | | | | | |
| P2-10-E3 | 0.007 | 0.249 | 35.57 | 0.017 | 0.153 | 9.00 |
| (Accession | | | | | | |
| No.) | | | | | | |
| P2-14-C6 | 0.006 | 0.353 | 58.8 | 0.054 | 0.143 | 2.65 |
| (Accession | | | | | | |
| No.) | | | | | | |
| P2-4-H5 | 0.214 | 0.058 | 3.69 | 0.217 | 0.065 | 3.34 |
| (Accession | | | | | | |
| No.) | | | | | | |
| P2-8-A3 | 0.184 | 0.095 | 1.94 | 0.227 | 0.065 | 3.49 |
| (Accession | | | | | | |
| No.) | | | | | | |
| P2-10-B8 | 0.101 | 0.055 | 1.84 | 0.121 | 0.029 | 4.17 |
| (Accession | | | | | | |
| No.) | | | | | | |
| P2-13-A9 | 0.114 | 0.004 | 28.5 | 0.213 | 0.035 | 6.09 |
| (Accession | | | | | | |
| No.) | | | | | | |

To derive monoclonal hybridoma lines, each colony was subcloned by limiting dilution. The resulting clonal lines derived from each parent colony were rescreened and ranked by O.D. 450 nm to determine the best clones. The top 10 antibody secreting clones were expanded and archived in liquid nitrogen storage. Cells were counted and ensured that the viability was at least 80%. Cells were prepared in subcloning media containing 10% FBS and 10% hybridoma cloning factor (bioVeris) in DMEM at 5 cells/ml (about 60 ml for 3 plates). Another set of the same cells was prepared at a concentration of ˜1.6 cells/ml (about 60 ml for 3 plates). Two hundred μl of cells were plated per well in a 96 well round bottom plate. One set of 3 plates contained 1 cell/well, and another contained, on average, 1 cell every 3 wells. After 10 days, cells were visible, and the subclones were tested for specificity. Cells of interest were expanded in a 24 well plate in 10% FBS DMEM containing 5% of hybridoma cloning factor.

The composition of each mAb was defined by determining the class of heavy and light chains, as well as the molecular weight, of each component. Isotyping was performed using the Immunopure monoclonal antibody isotyping kit I (Pierce) according to the manufacturer's instructions. The molecular weight of heavy and light chains was determined using the Experion automated electrophoresis system from Bio-Rad. The Experion system automatically performs the multiple steps of gel-based electrophoresis: separation, staining, destaining, band detection, imaging, and data analysis. The results of these analyses are shown in Table 9, which shows the physical characterization of CDr-HSD and CDs-HSD specific monoclonal antibodies. Identification of both heavy and light chains was performed using the Immunopure monoclonal antibody isotyping kit I (Pierce), and molecular weights (in kD) were determined using the Experion automated electrophoresis system (Bio-Rad).

TABLE 9

| Clone ID | | | | | |
|---|---|---|---|---|---|
| Accession | Light chain | | Heavy chain | | Whole IgG |
| No. | Subtype | Mol. Wt. | Subclass | Mol. Wt. | Mol. Wt. |
| P1-5-F11 | kappa | — | IgG2b | — | — |
| (Accession | | | | | |
| No.) | | | | | |
| P1-14-A2 | Kappa/ | — | IgG1 | — | — |
| (Accession | lambda | | | | |
| No.) | | | | | |
| P1-17-E4 | Kappa | — | IgG1 | — | — |
| (Accession | | | | | |
| No.) | | | | | |
| P1-18-C12 | Kappa | — | IgG2b | — | — |
| (Accession | | | | | |
| No.) | | | | | |
| P1-20-B7 | Kappa | — | IgG1 | — | — |
| (Accession | | | | | |
| No.) | | | | | |
| P1-23-F7 | Kappa | — | IgG2b | — | — |
| (Accession | | | | | |
| No.) | | | | | |
| P2-1-E8 | Kappa | — | IgG1 | — | — |
| (Accession | | | | | |
| No.) | | | | | |
| P2-10-E3 | Kappa | — | IgG2a | — | — |
| (Accession | | | | | |
| No.) | | | | | |
| P2-14-C6 | Kappa | — | IgG1 | — | — |
| (Accession | | | | | |
| No.) | | | | | |
| P2-4-H5 | Kappa | — | IgG2b | — | — |
| (Accession | | | | | |
| No.) | | | | | |
| P2-8-A3 | Kappa | — | IgG2b | — | — |
| (Accession | | | | | |
| No.) | | | | | |
| P2-10-B8 | Kappa | — | IgG2b | — | — |
| (Accession | | | | | |
| No.) | | | | | |
| P2-13-A9 | kappa | — | IgG1 | — | — |
| (Accession | | | | | |
| No.) | | | | | |

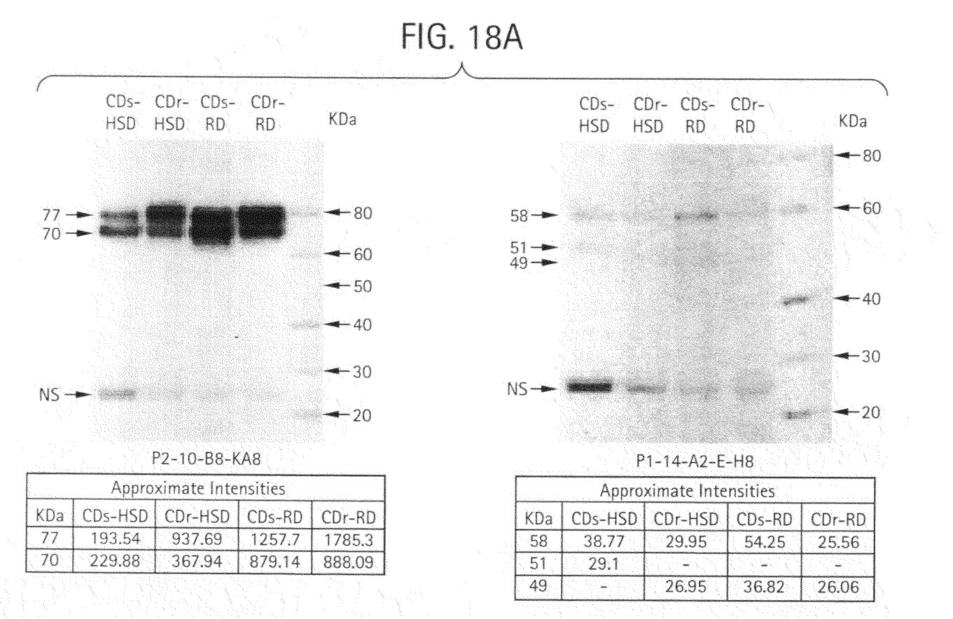

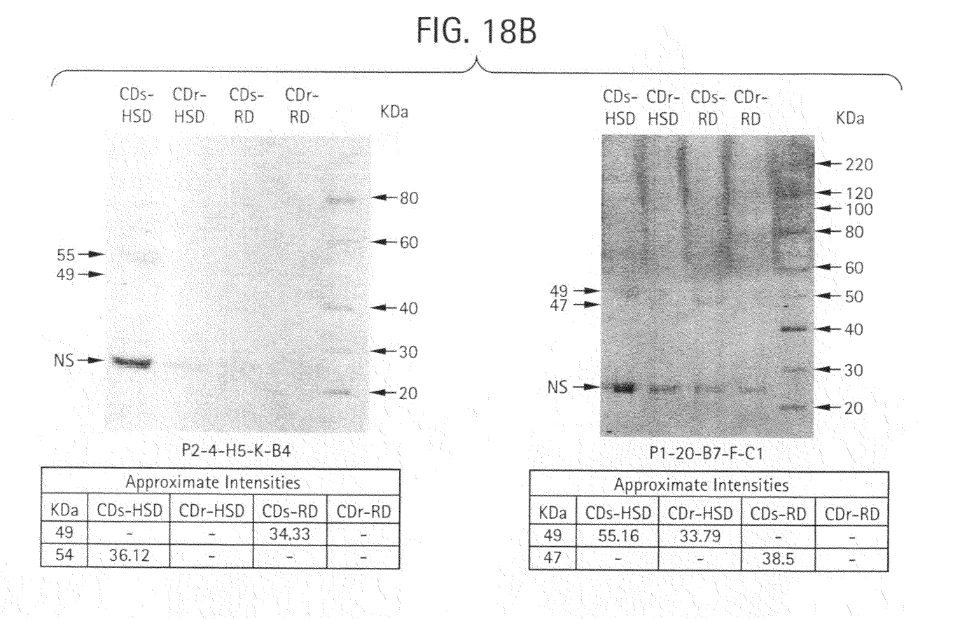

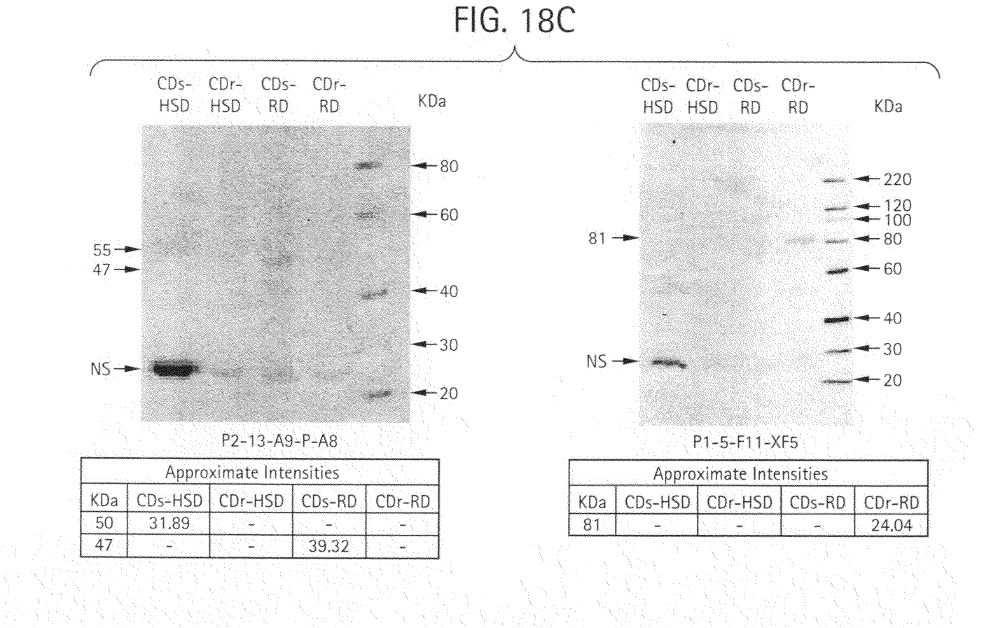

To determine the specific antigen for each clone, each mAb was tested by Western Blotting to ascertain the molecular weight of the corresponding antigen. Data obtained from reactive clones is shown in FIGS. 18A-18C.

To purify the antigen specific for P2-10-B8-KA8, an immunoprecipitation was performed. Specific antibody was bound to Protein G beads and used to pan for antigen from CDr-HSD pancreatic extract containing 6 mg of total protein. In an Eppendorf tube, CDR-HSD pancreatic extract was centrifuged for 5 minutes at 13,000 rpm, and the deposit on the top of the extract was removed. Without removing any of the pellet, 6 mg of extract was transferred to 3 clean centrifuge tubes and the volume adjusted 1 ml by addition of T-per buffer. To tube 1, 100 μg of purified P2-10-B8-KA8 was added to the diluted sample, 200 μg of purified P2-10-B8-KA8 was added to tube 2, and 300 μg of purified P2-10-B8-KA8 was added to tube 3. The tubes were rotated at 4° C. overnight.

Protein G beads slurry (1 ml) were centrifuged for 3 minutes at 500×g in an Eppendorf centrifuge, and washed twice with pre-chilled T-per buffer by diluting the beads 1:1 with the buffer. The slurry (200 μl) was transferred to each tube containing the antibody-antigen mixture. A control tube was set up by preparing a tube with 200 μl of slurry in 1 ml of T-Per buffer and 300 μg of antibody. The tubes were rotated at 4° C. for 2 hours. Thereafter, the beads were washed twice using pre-chilled T-per buffer (centrifuged at 500×g for 3 minutes) and the supernatants retained. After one final wash in cold PBS, the supernatant was removed as much as possible and 100 μl of 2× sample buffer (Pierce 5× loading buffer: 200 μl of loading buffer, 100 μl of reducing agent, complete with 200 μl of water) was added. The samples were boiled for 5 minutes at 95° C. and subsequently cooled on ice for 5 minutes. After spinning the samples for 3 minutes, each sample was loaded in an amount of 20 μl per lane on a 4-12% SDS-PAGE mini gel for electrophoresis.

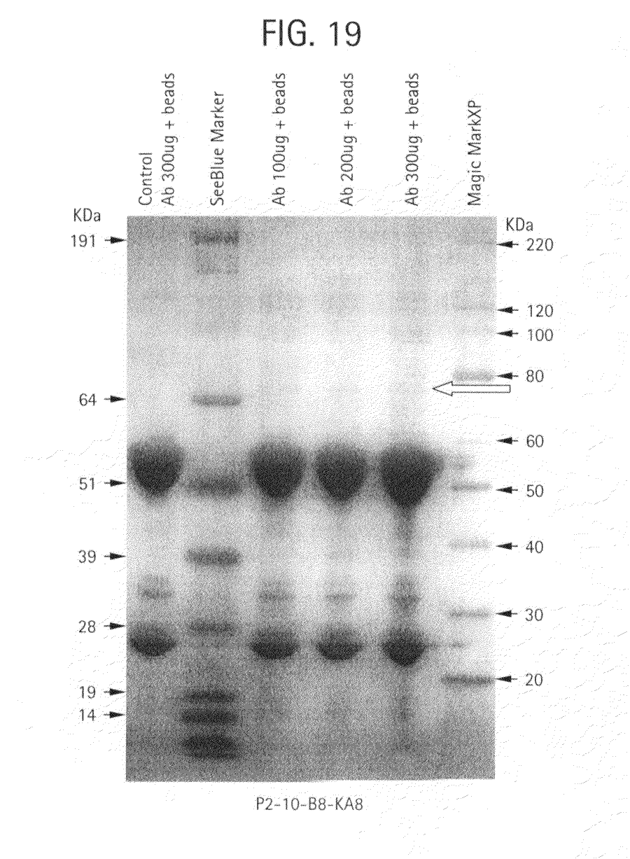

Following precipitation, several bands were visible on the gel after staining for total protein with Coomassie. A faint doublet band was observed in the molecular weight range of 70 to 80 kD (see FIG. 19). The doublet was confirmed to be the bands of interest by probing a Western Blot prepared from a similar gel with the same mAb (data not shown). The doublet bands were excised individually from the SDS-PAGE gel and submitted for identification by mass spectrometry. An positive identification of the lower band as calnexin was made. Calnexin is a molecular chaperone associated with the endoplasmic reticulum.

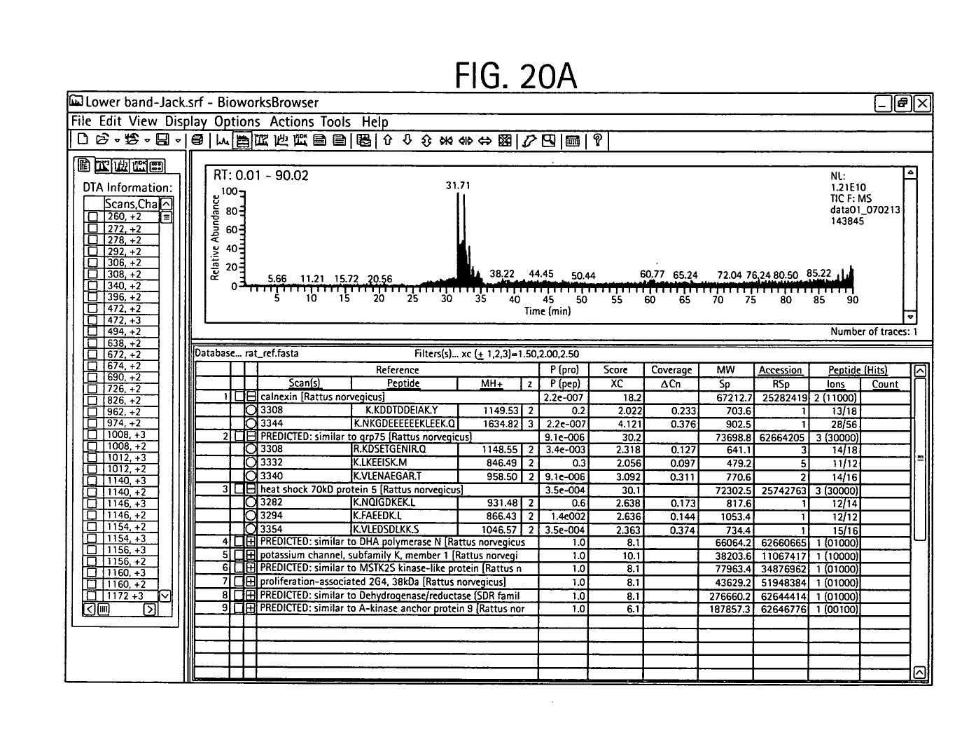

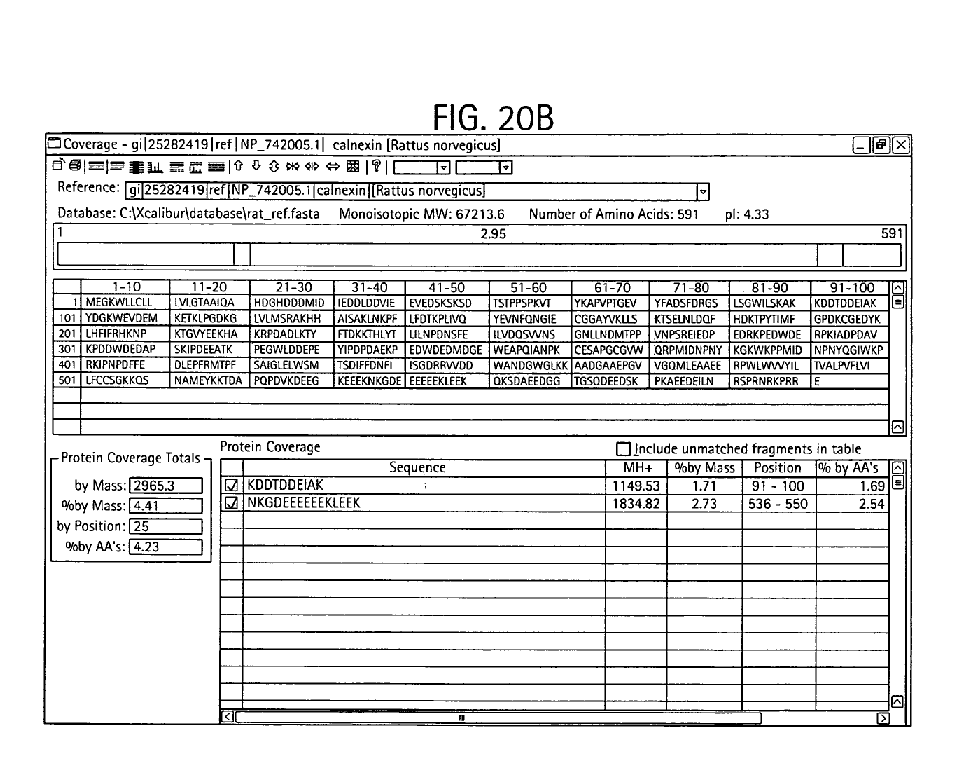

Calnexin is a 90 kD integral protein of the endoplasmic reticulum (ER). It consists of a large (50 kD) N-terminal calcium-binding lumenal domain, a single transmembrane helix and a short (90 residues), acidic cytoplasmic tail. Calnexin belongs to a family of proteins known as “chaperones,” which are characterized by their main function of assisting protein folding and quality control, ensuring that only properly folded and assembled proteins proceed further along the secretory pathway. The function of calnexin is to retain unfolded or unassembled N-linked glycoproteins in the endoplasmic reticulum. Calnexin binds only those N-glycoproteins that have GlcNAc2Man9Glc1 oligosaccharides. Oligosaccharides with three sequential glucose residues are added to asparagine residues of the nascent proteins in the ER. The monoglucosylated oligosaccharides that are recognized by calnexin result from the trimming of two glucose residues by the sequential action of two glucosidases, I and II. Glucosidase II can also remove the third and last glucose residue. If the glycoprotein is not properly folded, an enzyme called UGGT will add the glucose residue back onto the oligosaccharide thus regenerating the glycoprotein ability to bind to calnexin. The glycoprotein chain which for some reason has difficulty folding up properly thus loiters in the ER, risking the encounter with MNS1 (a-mannosidase), which eventually sentences the underperforming glycoprotein to degradation by removing its mannose residue. ATP and Ca²⁺ are two of the cofactors involved in substrate binding for calnexin. FIGS. 20A and 20B are screen shots depicting the read-out of the MS spectrograms identifying the protein of interest as calnexin.

### Example 4

### Microarray Analysis of Gene Expression in Tissues from Cohen Type 2 Diabetic Rats

The microarray data were analyzed through Phase I and Phase II analyses. Phase I is based on the processed data from Gene Logic. Phase II corresponds to data analysis using GeneSpring GX. Additional criteria including statistics, signaling pathways and clustering were used for the analyses.

The microarray results from Gene Logic (Phase I) that were derived from comparisons of pancreatic total RNA of Cohen Type 2 Diabetes rats (CDs-HSD, CDr-HSD) were analyzed using MAS5.0 software from Affymetrix, Inc. The global gene expression analysis showed that there were 1178 genes upregulated in CDr-HSD and 803 genes were downregulated in compared to CDs-HSD. Many of these transcripts are involved in several signaling pathways related to Type 2 Diabetes such as insulin signaling, beta-cell dysfunction and lipid and glucose metabolisms. Also, several serpin family members (serine proteinase inhibitors) are expressed differently in the two models. Table 10 provides a summary of the data derived from Gene Logic, wherein changes greater than 3-fold were observed.

TABLE 10

| | Upregulated | Downregulated |
|---|---|---|
| | genes | genes |
| | CDR-HS vs. | CDR-HS vs. |
| Signaling Pathways | CDS-HS | CDS-HS |
| Insulin signaling | 39 | 41 |
| β cell dysfunction (apoptosis, survival) | 17 | 6 |
| Inflammation and immune system | 5 | 92 |
| Mitochondrial dysfunction and reactive | 20 | 8 |
| oxygen species | | |
| Lipid and glucose metabolisms | 17 | 13 |
| proteinase and proteinase inhibitors | 28 | 17 |
| Amino acid, nucleic acid transporters and | 13 | 9 |
| metabolisms | | |
| Potassium channels | 3 | 6 |
| ER and Golgi body related genes | 8 | 8 |
| Other unclassified genes | 1028 | 603 |
| Total | 1178 | 803 |

Phase II data analysis was performed using GeneSpring GX, which used normalized data (ratio=transcript signal/control signal) to improve cross-chip comparison. GeneSpring GX allows for gene lists to be filtered according to genes exhibiting a 2-fold or 3-fold change in the expression levels. GeneSpring GX also comprises statistical algorithms, such as ANOVA, Post-Hoc Test, and Cross-Gene Error Modeling, as well as gene clustering algorithms like Gene Tree, K-mean clustering, and Self-Organizing Map (SOM) clustering. GeneSpring GX also has the ability to integrate with pathways that are published in the art, such as the Kyoto Encyclopedia of Genes and Genomes (“KEGG pathways”) and Gen Map Annotator and Pathway Profiler (GenMAPP).

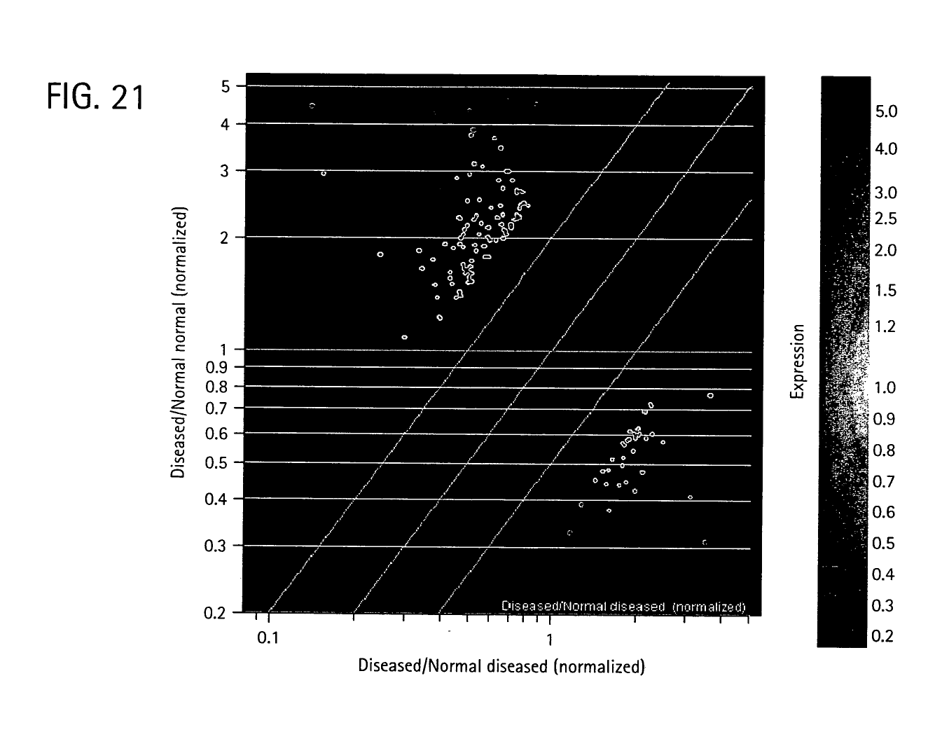

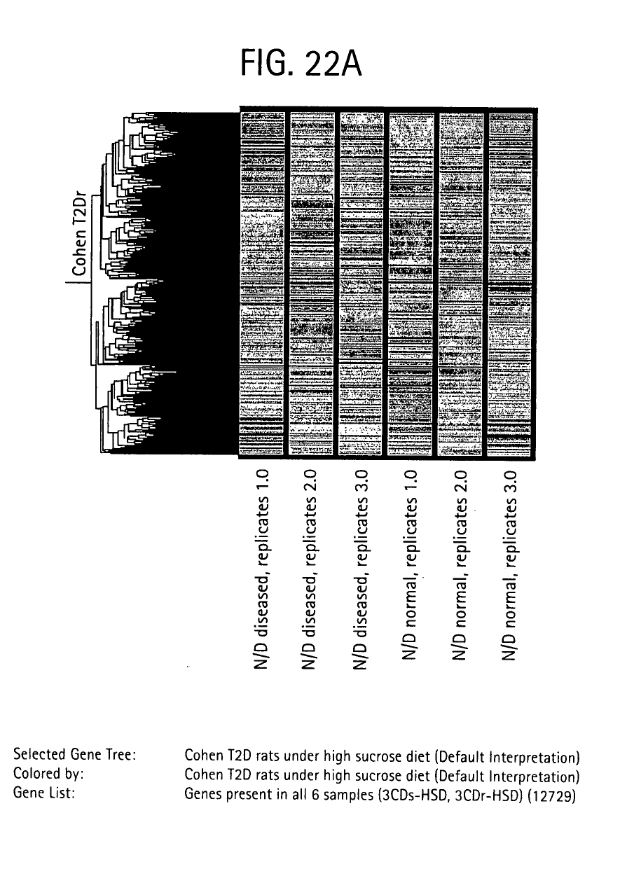

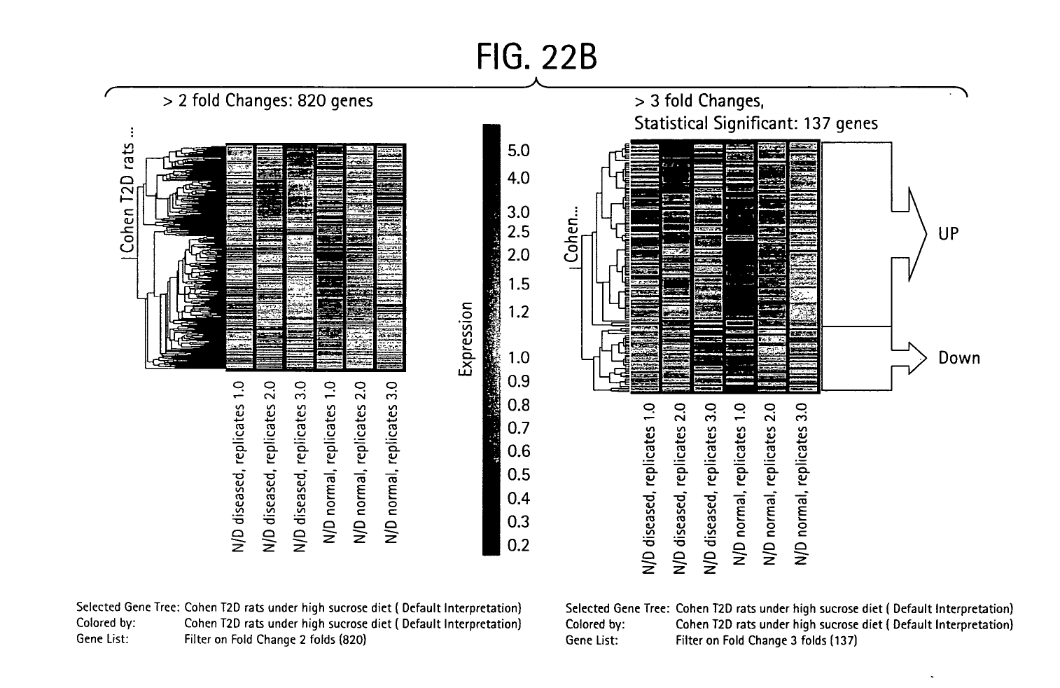

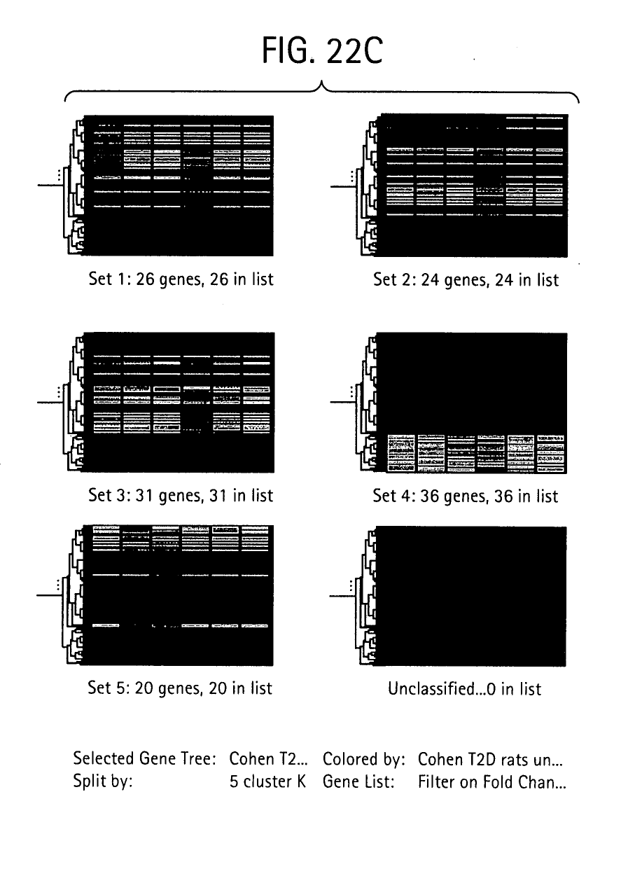

The microarray results analyzed by GeneSpring GX show that among the transcripts with changes higher than three fold in the two groups, 137 transcripts have a p-value of less than 0.05. These genes are involved in several signaling pathways such as the insulin signaling pathway, serpin protein family, basic metabolism, pancreas function and inflammation. FIG. 21 shows a scatter plot of differentially expressed genes. The 137 transcripts whose levels show a change of three-fold or higher are shown in FIG. 22B and are also grouped in Tables 11 and 12.

TABLE 11
Upregulated genes (Total = 101 Transcripts)

| Common | UniGene | Description |
|---|---|---|
| Reg3a | Rn.11222 | Regenerating islet-derived 3 alpha |
| LOC680945 | Rn.1414 | Similar to stromal cell-derived factor 2-like 1 |
| Pap | Rn.9727 | Pancreatitis-associated protein |
| Ptf1a | Rn.10536 | Pancreas specific transcription factor, 1a |
| Mat1a | Rn.10418 | Methionine adenosyltransferase I, alpha |
| Nupr1 | Rn.11182 | Nuclear protein 1 |
| | Rn.128013 | unknown cDNA |
| Chac1_predicted | Rn.23367 | ChaC, cation transport regulator-like 1 (E. coli) (predicted) |
| Slc7a3 | Rn.9804 | Solute carrier family 7 (cationic amino acid transporter, y+ |
| | | system), member 3 |
| LOC312273 | Rn.13006 | Trypsin V-A |
| | Rn.47821 | Transcribed locus |
| Ptger3 | Rn.10361 | Prostaglandin E receptor 3 (subtype EP3) |
| RGD1562451_predicted | Rn.199400 | Similar to Pabpc4_predicted protein (predicted) |
| RGD1566242_predicted | Rn.24858 | Similar to RIKEN cDNA 1500009M05 (predicted) |
| Cyp2d26 | Rn.91355 | Cytochrome P450, family 2, subfamily d, polypeptide 26 |
| | Rn.17900 | similar to aldehyde dehydrogenase 1 family, member L2 |
| LOC286960 | Rn.10387 | Preprotrypsinogen IV |
| Gls2 | Rn.10202 | Glutaminase 2 (liver, mitochondrial) |
| Nme2 | Rn.927 | Expressed in non-metastatic cells 2 |
| | Rn.165714 | Transcribed locus |
| P2rx1 | Rn.91176 | Purinergic receptor P2X, ligand-gated ion channel, 1 |
| Pdk4 | Rn.30070 | Pyruvate dehydrogenase kinase, isoenzyme 4 |
| Amyl | Rn.116361 | Amylase 1, salivary |
| Cbs | Rn.87853 | Cystathionine beta synthase |
| Mte1 | Rn.37524 | Mitochondrial acyl-CoA thioesterase 1 |
| Spink1 | Rn.9767 | Serine protease inhibitor, Kazal type 1 |
| Gatm | Rn.17661 | Glycine amidinotransferase (L-arginine:glycine |
| | | amidinotransferase) |
| Tmed6_predicted | Rn.19837 | Transmembrane emp24 protein transport domain |
| | | containing 6 (predicted) |
| Tff2 | Rn.34367 | Trefoil factor 2 (spasmolytic protein 1) |
| Hsd17b13 | Rn.25104 | Hydroxysteroid (17-beta) dehydrogenase 13 |
| | Rn.11766 | imilar to LRRGT00012 [Rattus norvegicus] |
| Gnmt | Rn.11142 | Glycine N-methyltransferase |
| Pah | Rn.1652 | Phenylalanine hydroxylase |
| Serpini2 | Rn.54500 | serine (or cysteine) proteinase inhibitor, clade I, member 2 |
| RGD1309615 | Rn.167687 | unknown cDNA |
| LOC691307 | Rn.79735 | Similar to leucine rich repeat containing 39 isoform 2 |
| Eprs | Rn.21240 | Glutamyl-prolyl-tRNA synthetase |
| Pck2_predicted | Rn.35508 | Phosphoenolpyruvate carboxykinase 2 (mitochondrial) |
| | | (predicted) |
| Chd2_predicted | Rn.162437 | Chromodomain helicase DNA binding protein 2 |
| | | (predicted) |
| | Rn.53085 | Transcribed locus |
| | Rn.12530 | Transcribed locus |
| NIPK | Rn.22325 | tribbles homolog 3 (Drosophila) |
| Slc30a2 | Rn.11135 | Solute carrier family 30 (zinc transporter), member 2 |
| Serpina10 | Rn.10502 | Serine (or cysteine) peptidase inhibitor, clade A, member |
| | | 10 |
| Cfi | Rn.7424 | Complement factor I |
| Cckar | Rn.10184 | Cholecystokinin A receptor |
| LOC689755 | Rn.151728 | Hypothetical protein LOC689755 |
| Bhlhb8 | Rn.9897 | Basic helix-loop-helix domain containing, class B, 8 |
| Anpep | Rn.11132 | Alanyl (membrane) aminopeptidase |
| Asns | Rn.11172 | Asparagine synthetase |
| Slc7a5 | Rn.32261 | Solute carrier family 7 (cationic amino acid transporter, y+ |
| | | system), member 5 |
| Usp43_predicted | Rn.12678 | Ubiquitin specific protease 43 (predicted) |
| Csnk1a1 | Rn.23810 | Casein kinase 1, alpha 1 |
| Pck2_predicted | Rn.35508 | Phosphoenolpyruvate carboxykinase 2 (mitochondrial) |
| | | (predicted) |
| Spink1 | Rn.9767 | Serine protease inhibitor, Kazal type 1 |
| Cml2 | Rn.160578 | Camello-like 2 |
| Pabpc4 | Rn.199602 | Transcribed locus |
| Gjb2 | Rn.198991 | Gap junction membrane channel protein beta 2 |
| Ngfg | Rn.11331 | Nerve growth factor, gamma |
| Clca2_predicted | Rn.48629 | Transcribed locus |
| RGD1565381_predicted | Rn.16083 | Similar to RIKEN cDNA 1810033M07 (predicted) |
| Qscn6 | Rn.44920 | Quiescin Q6 |
| Cldn10_predicted | Rn.99994 | Claudin 10 (predicted) |
| Spink3 | Rn.144683 | Serine protease inhibitor, Kazal type 3 |
| LOC498174 | Rn.163210 | Similar to NipSnap2 protein (Glioblastoma amplified |
| | | sequence) |
| | Rn.140163 | similar to Methionine-tRNA synthetase [Rattus |
| | | norvegicus] |
| Cyr61 | Rn.22129 | Cysteine rich protein 61 |
| RGD1307736 | Rn.162140 | Similar to Hypothetical protein KIAA0152 |
| Ddit3 | Rn.11183 | DNA-damage inducible transcript 3 |
| Reg1 | Rn.11332 | Regenerating islet-derived 1 |
| Eprs | Rn.21240 | Glutamyl-prolyl-tRNA synthetase |
| NIPK | Rn.22325 | cDNA clone RPCAG66 3′ end, mRNA sequence. |
| Eif4b | Rn.95954 | Eukaryotic translation initiation factor 4B |
| Spink1 | Rn.9767 | Serine protease inhibitor, Kazal type 1 |
| Rnase4 | Rn.1742 | Ribonuclease, RNase A family 4 |
| Cebpg | Rn.10332 | CCAAT/enhancer binding protein (C/EBP), gamma |
| siat7D | Rn.195322 | Alpha-2,6-sialyltransferase ST6GalNAc IV |
| Herpud1 | Rn.4028 | Homocysteine-inducible, ubiquitin-like domain member 1 |
| | | unknown rat cDNA |
| Gcat | Rn.43940 | Glycine C-acetyltransferase (2-amino-3-ketobutyrate- |
| | | coenzyme A ligase) |
| RGD1562860_predicted | Rn.75246 | Similar to RIKEN cDNA 2310045A20 (predicted) |
| Hspa9a_predicted | Rn.7535 | Heat shock 70 kD protein 9A (predicted) |
| Dbt | Rn.198610 | Dihydrolipoamide branched chain transacylase E2 |
| Bspry | Rn.53996 | B-box and SPRY domain containing |
| Fut1 | Rn.11382 | Fucosyltransferase 1 |
| Rpl3 | Rn.107726 | Ribosomal protein L3 |
| | Rn.22481 | similar to NP_083620.1 acylphosphatase 2, muscle type |
| | | [Mus musculus] |
| | | unknow rat cDNA |
| Vldlr | Rn.9975 | Very low density lipoprotein receptor |
| RGD1311937_predicted | Rn.33652 | Similar to hypothetical protein MGC17299 (predicted) |
| RGD1563144_predicted | Rn.14702 | Similar to EMeg32 protein (predicted) |
| | Rn.43268 | Transcribed locus |
| pre-mtHSP70 | Rn.7535 | 70 kD heat shock protein precursor; |
| Ddah1 | Rn.7398 | Dimethylarginine dimethylaminohydrolase 1 |
| RGD1307736 | Rn.162140 | Similar to Hypothetical protein KIAA0152 |
| RAMP4 | Rn.2119 | Ribosome associated membrane protein 4 |
| Ptger3 | Rn.10361 | Prostaglandin E receptor 3 (subtype EP3) |
| | Rn.169405 | Transcribed locus |
| Ccbe1_predicted | Rn.199045 | Collagen and calcium binding EGF domains 1 (predicted) |
| Dnajc3 | Rn.162234 | DnaJ (Hsp40) homolog, subfamily C, member 3 |
| Mtac2d1 | Rn.43919 | Membrane targeting (tandem) C2 domain containing 1 |

TABLE 12
Downregulated genes (Total = 36 transcripts)

| Common | UniGene | Description |
|---|---|---|
| RGD1563461_predicted | Rn.199308 | Transcribed locus |
| Gimap4 | Rn.198155 | GTPase, IMAP family member 4 |
| S100b | Rn.8937 | S100 protein, beta polypeptide |
| Klf2_predicted | Rn.92653 | Kruppel-like factor 2 (lung) (predicted) |
| RGD1309561_predicted | Rn.102005 | Similar to hypothetical protein FLJ31951 (predicted) |
| NAP22 | Rn.163581 | Transcribed locus |
| Sfrs3_predicted | Rn.9002 | Splicing factor, arginine/serine-rich 3 (SRp20) |
| | | (predicted) |
| | Rn.6731 | Transcribed locus |
| Cd53 | Rn.31988 | CD53 antigen |
| RGD1561419_predicted | Rn.131539 | Similar to RIKEN cDNA 6030405P05 gene (predicted) |
| Il2rg | Rn.14508 | Interleukin 2 receptor, gamma |
| LOC361346 | Rn.31250 | Similar to chromosome 18 open reading frame 54 |
| Cd38 | Rn.11414 | CD38 antigen |
| Klf2_predicted | Rn.92653 | Kruppel-like factor 2 (lung) (predicted) |
| Plac8_predicted | Rn.2649 | Placenta-specific 8 (predicted) |
| LOC498335 | Rn.6917 | Similar to Small inducible cytokine B13 precursor |
| | | (CXCL13) |
| Igfbp3 | Rn.26369 | Insulin-like growth factor binding protein 3 |
| Ptprc | Rn.90166 | Protein tyrosine phosphatase, receptor type, C |
| RT1-Aw2 | Rn.40130 | RT1 class Ib, locus Aw2 |
| Rac2 | Rn.2863 | RAS-related C3 botulinum substrate 2 |
| | Rn.9461 | Transcribed locus |
| Fos | Rn.103750 | FBJ murine osteosarcoma viral oncogene homolog |
| Arhgdib | Rn.15842 | Rho, GDP dissociation inhibitor (GDI) beta |
| Sgne1 | Rn.6173 | Secretory granule neuroendocrine protein 1 |
| Lck_mapped | Rn.22791 | Lymphocyte protein tyrosine kinase (mapped) |
| Fcgr2b | Rn.33323 | Fc receptor, IgG, low affinity IIb |
| Slfn8 | Rn.137139 | Schlafen 8 |
| Rab8b | Rn.10995 | RAB8B, member RAS oncogene family |
| | Rn.4287 | unknown cDNA |
| RGD1306939 | Rn.95357 | Similar to mKIAA0386 protein |
| Tnfrsf26_predicted | Rn.162508 | Tumor necrosis factor receptor superfamily, member 26 |
| | | (predicted) |
| Ythdf2_predicted | Rn.21737 | YTH domain family 2 (predicted) |
| RGD1359202 | Rn.10956 | Similar to immunoglobulin heavy chain 6 (Igh-6) |
| RGD1562855_predicted | Rn.117926 | Similar to Ig kappa chain (predicted) |
| Igha_mapped | Rn.109625 | Immunoglobulin heavy chain (alpha polypeptide) |
| | | (mapped) |
| Ccl21b | Rn.39658 | Chemokine (C-C motif) ligand 21b (serine) |

Gene Tree gene clustering analysis, represented in FIG. 22A, shows the 12,729 genes that are present in all six samples. As discussed above, 820 genes showed 2-fold changes in expression, while 137 genes showed 3-fold changes in expression, and a Gene Tree representation is shown in FIG. 22B. Of the 137 genes that showed 3-fold changes, K-mean clustering analysis further divided these 137 genes into 5 sets, based on the greatest similarities between the genes within the sets (FIG. 21C). These 5 sets are designated “Up-1”, “Up-2”, “Up-3”, “Up-4”, and “Up-5” and are summarized in Tables 13-17 below.

TABLE 13
Up-1
Total Genes: 91

| Common | Description | Fold Changes |
|---|---|---|
| Reg3a | Regenerating islet-derived 3 alpha | 75.08 |
| LOC680945 | Similar to stromal cell-derived factor 2-like 1 | 32.31 |
| Pap | Pancreatitis-associated protein | 19.53 |
| Ptf1a | Pancreas specific transcription factor, 1a | 11.59 |
| Mat1a | Methionine adenosyltransferase I, alpha | 8.67 |
| Nupr1 | Nuclear protein 1 | 7.53 |
| | Unknown cDNA | 7.52 |
| Chac1_predicted | ChaC, cation transport regulator-like 1 (E. coli) | 7.41 |
| | (predicted) | |
| Slc7a3 | Solute carrier family 7 (cationic amino acid | 6.68 |
| | transporter, y+ system), member 3 | |
| LOC312273 | Trypsin V-A | 6.38 |
| | Transcribed locus | 6.08 |
| Ptger3 | Prostaglandin E receptor 3 (subtype EP3) | 6.01 |
| RGD1562451_predicted | Similar to Pabpc4_predicted protein | 5.88 |
| | (predicted) | |
| RGD1566242_predicted | Similar to RIKEN cDNA 1500009M05 | 5.62 |
| | (predicted) | |
| Cyp2d26 | Cytochrome P450, family 2, subfamily d, | 5.59 |
| | polypeptide 26 | |
| | Similar to aldehyde dehydrogenase 1 family, | 5.37 |
| | member L2 (Canis familiaris) | |
| LOC286960 | Preprotrypsinogen IV | 5.19 |
| Gls2 | Glutaminase 2 (liver, mitochondria) | 5.10 |

TABLE 14
Up-2
Total Genes: 91

| | | Fold |
|---|---|---|
| Common | Description | Changes |
| | Transcribed locus | 4.92 |
| P2rx1 | Purinergic receptor P2X, ligand-gated ion | 4.85 |
| | channel, 1 | |
| Pdk4 | Pyruvate dehydrogenase kinase, | 4.72 |
| | isoenzyme 4 | |
| Amy1 | Amylase 1, salivary | 4.70 |
| Cbs | Cystathionine beta synthase | 4.67 |
| Mte1 | Mitochondrial acyl-CoA thioesterase 1 | 4.49 |
| Spink1 | Serine protease inhibitor, Kazal type 1 | 4.43 |
| Gatm | Glycine amidinotransferase (L- | 4.40 |
| | arginine:glycine amidinotransferase) | |
| Tmed6_predicted | Transmembrane emp24 protein transport | 4.38 |
| | domain containing 6 (predicted) | |
| Tff2 | Trefoil factor 2 (spasmolytic protein 1) | 4.36 |
| Hsd17b13 | Hydroxysteroid (17-beta) dehydrogenase | 4.34 |
| | 13 | |
| | Similar to LRRGT00012 (Rattus | 4.30 |
| | norvegicus) | |
| Gnmt | Glycine N-methyltransferase | 4.30 |
| Pah | Phenylalanine hydroxylase | 4.29 |
| Serpini2 | Serine (or cysteine) proteinase inhibitor, | 4.28 |
| | clade I, member 2 | |
| RGD1309615 | Unknown cDNA | 4.16 |
| LOC691307 | Similar to leucine rich repeat | 4.12 |
| | containing 39 isoform 2 | |
| Eprs | Glutamyl-prolyl-tRNA synthetase | 4.03 |
| Pck2_predicted | Phosphoenolpyruvate carboxykinase 2 | 4.01 |
| | (mitochondrial)(predicted) | |

TABLE 15
Up-3
Total Genes: 91

| Common | Description | Fold Changes |
|---|---|---|
| | Transcribed locus | 3.97 |
| | Transcribed locus | 3.96 |
| Slc30a2 | Solute carrier family 20 (zinc transporter), | 3.77 |
| | member 2 | |
| Serpina10 | Serine (or cysteine) peptidase inhibitor, clade | 3.77 |
| | A, member 10 | |
| Cfi | Complement factor 1 | 3.69 |
| Cckar | Cholecystokinin A receptor | 3.68 |
| LOC689755 | Hypothetical protein LOC 689755 | 3.68 |
| Bhlhb8 | Basic helix-loop-helix domain containing, | 3.66 |
| | class B, 8 | |
| Anpep | Alanyl (membrane) aminopeptidase | 3.65 |
| Asns | Asparagine synthetase | 3.65 |
| Usp43_predicted | Ubiquitin specific protease 43 (predicted) | 3.62 |
| Slc7a5 | Solute carrier family 7 (carionic amino acid | 3.62 |
| | transporter, y+ system), member 5 | |
| Csnk1a1 | Casein kinase 1, alpha 1 | 3.58 |
| Cml2 | Camello-like 2 | 3.51 |
| Pabpc4 | Transcribed locus | 3.50 |
| Gjb2 | Gap junction membrane channel protein beta 2 | 3.49 |
| Ngfg | Nerve growth factor, gamma | 3.47 |
| Clca2_predicted | Transcribed locus | 3.46 |
| RGD1565381_predicted | Similar to RIKEN cDNA 1810033M07 | 3.42 |
| | (predicted) | |
| Qscn6 | Quiescin Q6 | 3.41 |

TABLE 16
Up-4
Total Genes: 91

| Common | Description | Fold Changes |
|---|---|---|
| Cldn10_predicted | Claudin 10 (predicted) | 3.40 |
| Spink3 | Serine protease inhibitor, Kazal type 3 | 3.38 |
| LOC498174 | Similar to NipSnap2 protein (glioblastoma | 3.36 |
| | amplified sequence) | |
| | Similar to methionine-tRNA synthetase | 3.35 |
| | (Rattus norvegicus) | |
| Cyr61 | Cysteine rich protein 61 | 3.33 |
| RGD1307736 | Similar to hypothetical protein KIAA0152 | 3.32 |
| Ddit3 | DNA-damage inducible transcript 3 | 3.32 |
| Reg1 | Regenerating islet-derived 1 | 3.22 |
| NIPK | Unknown cDNA | 3.19 |
| Eif4b | Eukaryotic translation initiation factor 4B | 3.17 |
| Rnase4 | Ribonuclease, RNase A family 4 | 3.16 |
| Cebpg | CCAAT/enhancer binding protein (C/EBP), | 3.16 |
| | gamma | |
| Siat7D | Alpha-2,6-sialyltransferase ST6GalNAc IV | 3.15 |
| Herpud1 | Homocysteine-inducible, ubiquitin-like | 3.15 |
| | domain member 1 | |
| Gcat | Glycine C-acetyltransferase (2-amino-3- | 3.13 |
| | ketobutyrate-coenzyme A ligase) | |
| RGD1562860_predicted | Similar to RIKEN cDNA 2310045A20 | 3.11 |
| | (predicted) | |
| Hspa9a_predicted | Heat shock 70 kDa protein 9A (predicted) | 3.10 |
| Dbt | Dihydrolipoamide branched chain transacylase | 3.10 |
| | E2 | |
| Bspry | B-box and SPRY domain containing | 3.10 |

TABLE 17
Up-5
Total Genes: 91

| Common | Description | Fold Changes |
|---|---|---|
| Fut1 | Fucosyltransferase 1 | 3.09 |
| Rpl3 | Ribosomal protein L3 | 3.08 |
| | Strongly similar to NP_083620.1 | 3.08 |
| | acylphosphatase 2, muscle type (Mus | |
| | musculus) | |
| Vldlr | Very low density lipoprotein receptor | 3.07 |
| RGD1311937_predicted | Similar to hypothetical protein MGC17299 | 3.04 |
| | (predicted) | |
| RGD1563144_predicted | Similar to EMeg32 protein (predicted) | 3.04 |
| | Transcribed locus | 3.04 |
| Ddah1 | Dimethylarginine dimethylaminohydrolase 1 | 3.03 |
| RAMP4 | Ribosome associated membrane protein 4 | 3.01 |
| | Transcribed locus | 3.01 |
| Ccbe1_predicted | Collagen and calcium binding EGF domains 1 | 3.01 |
| | (predicted) | |
| Dnajc | DnaJ (Hsp40) homolog, subfamily C, member 3 | 3.00 |
| Mtac2d1 | Membrane targeting (tandem) C2 domain | 3.00 |
| | containing 1 | |

Two additional sets, named “Down-1” and “Down-2” represent genes that were found by GeneSpring GX analysis to be downregulated in the Cohen diabetic rat samples. The following Tables 18 and 19 summarize the results obtained in the “Down-1” and “Down-2” sets.

TABLE 18
Down-1
Total Genes: 35 genes

| | | Fold |
|---|---|---|
| Common | Description | Change |
| Ccl21b | Chemokine (C-C motif) ligand 21b | |
| | (serine) | |
| Igha_mapped | Immunoglobulin heavy chain (alpha | |
| | polypeptide) (mapped) | |
| RGD1562855_predicted | Similar to Ig kappa chain (predicted) | |
| RGD1359202 | Similar to immunoglobulin heavy | |
| | chain 6 (Igh-6) | |
| Ythdf2_predicted | YTH domain family 2 (predicted) | |
| Tnfrsf26_predicted | Tumor necrosis factor receptor | |
| | superfamily, member 26 | |
| | (predicted) | |
| RGD1306939 | Similar to mKIAA0386 protein | |
| | Unknown cDNA | |
| Rab8b | RAB8B, member RAS oncogene | |
| Slfn8 | family | |
| Fcgr2b | | |
| Lck_mapped | | |
| Sgne1 | | |
| Fos | | |
| Arhgdib | | |
| Rac2 | | |

TABLE 19
Down-2
Total Genes: 35 genes

| Common | Description | Fold Changes |
|---|---|---|
| RT1-Aw2 | Rt1 class Ib, locus Aw2 | 3.39 |
| Ptprc | Protein tyrosine phosphatase, receptor type, C | 3.39 |
| Igfbp3 | Insulin-like growth factor binding protein 3 | 3.37 |
| LOC498335 | Similar to small inducible cytokine B13 | 3.27 |
| | precursor (CXCL13) | |
| Plac8_predicted | Placenta-specific 8 (predicted) | 3.25 |
| Cd38 | CD38 antigen | 3.24 |
| LOC361346 | Similar to chromosome 18 open reading frame | 3.24 |
| | 54 | |
| RGD1561419_predicted | Similar to RIKEN cDNA 6030405P05 | 3.19 |
| | (predicted) | |
| Il2rg | Interleukin 2 receptor, gamma (severe | 3.19 |
| | combined immunodeficiency) | |
| Cd53 | CD53 antigen | 3.18 |
| | Transcribed locus | 3.16 |
| Sfrs3_predicted | Splicing factor, arginine/serine-rich 3 (SRp20) | 3.15 |
| | (predicted) | |
| RGD1309561_predicted | Similar to hypothetical protein FLJ31951 | 3.13 |
| | (predicted) | |
| NAP22 | Transcribed locus | 3.13 |
| Klf2_predicted | Kruppel-like factor 2 (lung) (predicted) | 3.11 |
| S100b | S100 protein, beta polypeptide | 3.08 |
| Gimap4 | GTPase, IMAP family member 4 | 3.07 |
| RGD1563461_predicted | Transcribed locus | 3.07 |

Finally, gene expression analyses obtained by microarray were confirmed using quantitative RT-PCR according to standard methods. The table below provides a summary of the genes of interest identified by microarray analysis and whose fold changes in expression were verified using Q-RT-PCR.

TABLE 20
Quantitative RT-PCR Analysis on Selected Genes

| Common | Genbank | Unigene | Description | Fold Change |
|---|---|---|---|---|
| | | | | Downregulated |
| Ccl2b | BI282920 | Rn.39658 | Chemokine (C-C motif) ligand 21b | 11.33 |
| | | | (serine) | |
| Tnfrsf26_predicted | BE098317 | Rn.162508 | Tumor necrosis factor receptor | 4.37 |
| | | | superfamily, member 26 (predicted) | |
| Igfbp3 | NM_012588 | Rn.26369 | Insulin-like growth factor binding | 3.37 |
| | | | protein 3 | |
| Il2rg | AI178808 | Rn.14508 | Interleukin 2 receptor, gamma (severe | 3.19 |
| | | | combined immunodeficiency) | |
| | | | | Upregulated |
| Reg3a | L10229 | Rn.11222 | Regenerating islet-derived 3 alpha | 75.08 |
| LOC680945 | BI275923 | Rn.1414 | Similar to stromal cell-derived factor | 32.31 |
| | | | 2-like 1 | |
| Ptf1a | NM_053964 | Rn.10536 | Pancreas specific transcription factor, | 11.59 |
| | | | 1a | |
| LOC312273 | AI178581 | Rn.13006 | Trypsin V-A | 6.38 |
| LOC286960 | X15679 | Rn.10387 | Preprotrypsinogen IV | 5.19 |
| Spink1 | NM_012674 | Rn.9767 | Serine protease inhibitor, Kazal type 1 | 4.43 |
| Serpini2 | NM_133612 | Rn.54500 | Serine (or cysteine) proteinase | 4.28 |
| | | | inhibitor, clade I, member 2 | |
| Serpina10 | NM_133617 | Rn.10502 | Serine (or cysteine) peptidase | 3.77 |
| | | | inhibitor, clade A, member 10 | |
| Spink3 | M27883 | Rn.144683 | Serine protease inhibitor, Kazal type 3 | 3.38 |
| Reg1 | NM_012641 | Rn.11332 | Regenerating islet-derived 1 | 3.22 |
| Eif4a | BI278814 | Rn.95954 | Eukaryotic translation initiation factor | 3.17 |
| | | | 4B | |
| Rpl3 | BG057530 | Rn.107726 | Ribosomal protein L3 | 3.08 |
| RAMP4 | AI103695 | Rn.2119 | Ribosome associated membrane | 3.01 |
| | | | protein 4 | |

The protein encoded by the CD53 gene is a member of the transmembrane 4 superfamily, also known as the tetraspanin family. Most of these members are cell-surface proteins that are characterized by the presence of four hydrophobic domains. The proteins mediate signal transduction events that play a role in the regulation of cell development, activation, growth and motility. This encoded protein is a cell surface glycoprotein that is known to complex with integrins. It contributes to the transduction of CD2-generated signals in T cells and natural killer cells and has been suggested to play a role in growth regulation. Familial deficiency of this gene has been linked to an immunodeficiency associated with recurrent infectious diseases caused by bacteria, fungi and viruses. Alternative splicing results in multiple transcript variants encoding the same protein. CD38 is a novel multifunctional ectoenzyme widely expressed in cells and tissues especially in leukocytes. CD38 also functions in cell adhesion, signal transduction and calcium signaling.

Microarray and quantitative PCR analyses were applied to identify the transcriptome changes in pancreatic and epididymal fat tissues of the two strains exposed to a regular diet (RD) or diabetogenic/high sucrose diet (HSD). Both pancreatic tissues and visceral fat tissue-epididymal fat tissue are deemed important primary tissues to study gene transcripts that may play a crucial role in the prediction, progression, and possibly prevention of the disease.

Total RNA was extracted from pancreatic and epididymal fat tissues from each of the strains (CDs, CDr) under regular diet (R^{D}) and diabetogenic diet (HSD). The transcriptome was then analyzed using the Rat Expression Arrays (Affymetrix) set 230 which contains oligonucleotide probes for over 30,000 transcripts. Three to five rats from each groups (CDs-RD, CDs-HSD, CDr-RD and CDr-HSD) were used for data analyses. The results were analyzed using GeneSpring GX (Agilent, Calif.). Expression of several selected transcripts was also confirmed by real-time PCR.

Transcriptome changes of pancreatic tissue were first analyzed via microarray. For this experiment three animals from each of the following groups CDr-HSD and CDs-HSD were analyzed. In CDr-HSD and CDs-HSD rats, eighty-two (82) transcripts show a change of three fold or higher when the two groups are compared (see Tables 21 and 22); nineteen (19) transcripts are downregulated (expression in CDr-HSD is decreased 3 fold or more; Table 22), and sixty-three (63) transcripts were upregulated (expression in CDr-HSD is increased 3 fold or more; Table 21). Fourteen of these transcripts were selected and their changes in the expression levels were confirmed by quantitative PCR. The quantitative PCR analyses validated the changes of expression observed by micorarray analyses.

TABLE 21
Upregulated transcripts expressed 3-fold in CDr-HSD rats

| | UniGene | UniGene | |
|---|---|---|---|
| Name | (rat) | (human) | Description and Gene Ontology |
| REG3G | Rn.11222 | Hs.447084 | Regenerating islet-derived 3 gamma |
| SDF2L1 | Rn.1414 | Hs.303116 | Endoplasmic reticulum stress-inducible gene |
| REG3A | Rn.9727 | Hs.567312 | Regenerating islet-derived 3 alpha |
| MAT1A | Rn.10418 | Hs.282670 | Methionine adenosyltransferase |
| NUPR1 | Rn.11182 | Hs.513463 | Nuclear protein 1 |
| CHAC1 | Rn.23367 | Hs.155569 | Cation transport regulator-like 1 |
| SLC7A3 | Rn.9804 | Hs.175220 | Solute carrier family 7, member 3 |
| PRSS3 | Rn.13006 | Hs.128013 | Protease serine 3 (mesotrypsin) |
| BF415056 | Rn.47821 | n/a | Unknown cDNA |
| PABPC4 | Rn.199400 | Hs.169900 | Ploy A binding protein, cytoplasmic 4 |
| CYP2D6 | Rn.91355 | Hs.648256 | Cytochrome P450, 2D6 |
| AI044556 | Rn.17900 | n/a | unknown |
| PRSS4 | Rn.10387 | Hs.128013 | Mesotrypsin preproprotein |
| GLS2 | Rn.10202 | Hs.212606 | Glutaminase 2 (liver, mitochondrial) |
| NME2 | Rn.927 | Hs.463456 | Nucleoside diphosphate kinase-B |
| P2RX1 | Rn.91176 | Hs.41735 | Purinergic receptor P2X, ligand-gated ion channel 1 |
| PDK4 | Rn.30070 | Hs.8364 | Pyruvate dehydrogenase kinase, isoenzyme 4 |
| AMY1A | Rn.116361 | Hs.484588 | Amylase 1A, 1B and 2A and 2B are closely related |
| CBS | Rn.87853 | Hs.533013 | Cytathionine beta synthase |
| MTE1 | Rn.37524 | Hs.446685 | Acyl-CoA thioesterase2 or mitochondrial acyl-CoA |
| | | | thioesterase |
| SPINK1 | Rn.9767 | Hs.407856 | Serine protease inhibitor, Kazal type 1, |
| GATM | Rn.17661 | Hs.75335 | Glycine amidinotransferase (L-arginine:glycine |
| | | | amidinotransferase) |
| TMED6 | Rn.19837 | Hs.130849 | Transmembrane emp24 protein transport domain |
| | | | containing 6 |
| TFF2 | Rn.34367 | Hs.2979 | Trefoil factor 2 (spasmolytic protein 1) |
| HSD17B13 | Rn.25104 | Hs.284414 | Hydroxysteriod (17-beta) dehydrogenase 13 |
| GNMT | Rn.11142 | Hs.144914 | Glycine N-methyltransferase |
| LRRGT00012 | Rn.11766 | n/a | unknown |
| PAH | Rn.1652 | Hs.652123 | Phenylalanine hydroxylase |
| SERPINI2 | Rn.54500 | Hs.445555 | Serine proteinase inhibitor clade I, member 2 |
| RGD1309615 | Rn.167687 | n/a | Similar to hypothetical protein XP_580018 |
| LRRC39 | Rn.79735 | Hs.44277 | Leucine repeat containing 39 |
| EPRS | Rn.21240 | Hs.497788 | Glutamyl-prolyl-tRNA synthetase |
| PCK2 | Rn.35508 | Hs.75812 | Phosphoenolpyruvate carboxykinase 2 (mitrochondria) |
| AA997640 | Rn.12530 | n/a | unknown |
| SERPINA10 | Rn.10502 | Hs.118620 | Serine peptidase inhibitor, clade A, member 10 |
| SLC30A2 | Rn.11135 | Hs.143545 | Solute carrier family 30 (zinc transporter), member 2 |
| CCKAR | Rn.10184 | Hs.129 | Cholecystokinin A receptor |
| BHLHB8 | Rn.9897 | Hs.511979 | Basic helix-loop-helix domain containing, class B, 8 |
| ANPEP | Rn.11132 | Hs.1239 | Alanyl aminopeptidase |
| ASNS | Rn.11172 | Hs.489207 | Asparagines synthetase |
| SLC7A5 | Rn.32261 | Hs.513797 | Solute carrier family 7 member 5 |
| PABPC4 | Rn.2995 | Hs.169900 | Poly (A) binding protein, cytoplasmic 4(inducible) |
| KLK1 | Rn.11331 | Hs.123107 | Kallikrein 1 |
| ERP27 | Rn.16083 | Hs.162143 | Endoplasmic reticulum protein 27 KDa |
| QSCN6 | Rn.44920 | Hs.518374 | Quiescin 6 |
| CLDN10 | Rn.99994 | Hs.534377 | Claudin10 |
| MARS | Rn.140163 | Hs.632707 | Methonine-tRNA synthetase |
| EIF4B | Rn.95954 | Hs.292063 | Eukaryotic translation initiation factor 4B |
| RNASE4 | Rn.1742 | Hs.283749 | Ribonuclease, Rnase A family 4 |
| ST6GALNAC4 | Rn.195322 | Hs.3972 | Alpha-2,6-sialytransferase ST6GALNAC 4 |
| HERPUD1 | Rn.4028 | Hs.146393 | Homocysteine-inducible, endoplasmic reticulum stress- |
| | | | inducible, ubiquitin-like domain member 1 |
| DBT | Rn.198610 | Hs.653216 | Dihydrolipoamide branched chain transferase E2 |
| FUT1 | Rn.11382 | Hs.69747 | Fucosyltransferase 1 |
| AL170755 | Rn.22481 | n/a | unknown |
| VLDLR | Rn.9975 | Hs.370422 | Very low density lipoprotein receptor |
| GNPNAT1 | Rn.14702 | Hs.478025 | Glucosamine phosphate N-acetyltransferase 1 |
| DDAH1 | Rn.7398 | Hs.379858 | Dimethylarginine dimethylaminohydrolase 1 |
| HSPA9 | Rn.7535 | Hs.184233 | Heat shock 70 Kda protein 9 |
| PTGER3 | Rn.10361 | Hs.445000 | Prostaglandin E receptor 3 |
| AW523490 | Rn.169405 | n/a | Unknown cNDA |
| RAMP4 | Rn.2119 | Hs.518326 | Ribosome associated membrane |
| MTAC2D1 | Rn.43919 | Hs.510262 | Membrane targeting 9tandem) C2 domain containing 1 |
| DNAJC3 | Rn.162234 | Hs.591209 | DnaJ homolog, subfamily C, member 3 |

TABLE 22
Downregulated transcripts showing 3-fold reduced in expression in CDr-HSD rats

| | UniGene | UniGene | |
|---|---|---|---|
| Name | (rat) | (human) | Description and Gene Ontology |
| CCL21 | Rn.39658 | Hs.57907 | chemokine (C-C motif) ligand 21b |
| IGHG1 | Rn.10956 | Hs.510635 | IGHG1 in human: immunoglobulin heavy constant |
| | | | gamma 1 |
| IGHM | Rn.201760 | Hs.510635 | IGHM: immunoglobulin heavy constant mu |
| Tnfrsf26 | Rn.162508 | n/a | Tumor necrosis factor receptor superfamily, member |
| | | | 26 |
| RGD1306939 | Rn.95357 | n/a | Unknown |
| CD32 | Rn.33323 | Hs.352642 | Fc receptor, IgG, low affinity IIb |
| LCK | Rn.22791 | Hs.470627 | Lymphocyte protein tyrosine kinase |
| SCG5 | Rn.6173 | Hs.156540 | Secretogranin V |
| ARHGD1B | Rn.15842 | Hs.504877 | Rho GDP dissociation inhibitor (GDI) beta |
| RAC2 | Rn.2863 | Hs.517601 | RAS-related C3 botulinum toxin substrate 2 |
| CD45 | Rn.90166 | Hs.192039 | Protein tyrosine phosphatase, receptor type |
| BAT3 | Rn.40130 | Hs.440900 | HLA-B associated transcript 3 |
| CD38 | Rn.11414 | Hs.479214 | CD38 antigen |
| CD132 | Rn.14508 | Hs.84 | Interleukin 2 receptor, gamma |
| ARHGAP30 | Rn.131539 | Hs.389374 | Rho GTPase activating protein 30 |
| CD53 | Rn.31988 | Hs.443057 | CD53 antigen |
| S100B | Rn.8937 | Hs.422181 | S100 calcium binding protein B |
| GIMAP4 | Rn.198155 | Hs.647101 | GTPase, IMAP family member4 |
| RGD1563461 | Rn.199308 | n/a | Unknown |

Given the changes observed in the pancreatic tissue and their consistency by both methods microarray analyses and quantitative PCR, changes in transcriptome levels in epidydimal fat tissue for all four groups of Cohen Diabetic rats were also analyzed. Comparisons among groups may lead to discovery of biomarkers used for either predisposition, progression, and resistance of Type 2 diabetes. For example, CDr-RD versus CDs-RD comparisons may indicate predisposition for Type 2 diabetes, while CDs-RD versus CDs-HSD comparisons may serve as a model for progression of the disease, and CDr-HSD versuss CDs-HSD comparisons may be used as a model for resistance against development of Type 2 diabetes.

Tissue samples from five animals from each of the above-mentioned groups were analyzed and the results are summarized herein. Two hundred (200) transcripts, eighty (80) known transcripts and one hundred and twenty (120) unknown transcripts were expressed only in CDs-HSD group, the group that develops Type 2 Diabetes. Twenty-five (25) transcripts with signal strengths (arbitrary fluorescence units) significantly greater than the background noise are listed in Table 23.

TABLE 23
Transcripts Expressed Only in CDs-HSD Rats

| | UniGene | |
|---|---|---|
| Name | (rat) | Description and Gene Ontology |
| RGD1306952 | Rn.64439 | Similar to Ab2-225 |
| Dmrt2 | Rn.11448 | Doublesex and mab-3 related transcription factor 2 |
| | | (predicted) |
| AA819893 | Rn.148042 | unknown cDNA |
| Gpr176 | Rn.44656 | G protein-coupled receptor 176 |
| Tmem45b | Rn.42073 | Transmembrane protein 45b |
| Nfkbil1 | Rn.38632 | Nuclear factor of kappa light polypeptide gene |
| | | enhancer in B-cells inhibitor-like 1 |
| Dctn2 | Rn.101923 | Dynactin 2 |
| Itpkc | Rn.85907 | Inositol 1,4,5-trisphosphate 3-kinase C |
| BM389613 | Rn.171826 | unknown cDNA |
| Prodh2 | Rn.4247 | Proline dehydrogenase (oxidase) 2 |
| BF288777 | Rn.28947 | unknown cDNA |
| Abi3 | Rn.95169 | ABI gene family, member 3 |
| Ring1 | Rn.116589 | Ring finger protein 1 |
| Adrbk1 | Rn.13010 | Adrenergic receptor kinase, beta 1 |
| AW531966 | Rn.8608 | unknown cDNA |
| RGD1560732 | Rn.100399 | Similar to LIM and senescent cell antigen-like domains |
| | | 1 (predicted) |
| Oxsr1 | Rn.21097 | Oxidative-stress responsive 1 (predicted) |
| MGC114531 | Rn.39247 | unknown cDNA |
| BF418465 | Rn.123735 | unknown cDNA |
| LOC690911 | Rn.25022 | Similar to Msx2-interacting protein (SPEN homolog) |
| Pex6 | Rn.10675 | Peroxisomal biogenesis factor 6 |
| RGD1311424 | Rn.57800 | Similar to hypothetical protein FLJ38348 (predicted) |
| AI013238 | Rn.135595 | unknown cDNA |
| BI288719 | Rn.45106 | unknown cDNA |
| Evpl | Rn.19832 | Envoplakin (predicted) |

The results of comparisons among the three groups are presented in Table 24 below. Among the genes differentially expressed for each of the models, there are several common transcripts.

TABLE 24
Results of microarray analyses in epididymal fat tissue.

| | CDr-HSD vs. | CDs-HSD vs. | CDr-RD vs. |
|---|---|---|---|
| Comparisons | CDs-HSD | CDs-RD | CDs-RD |
| Type of model | Resistance | Progression | Predisposition |
| >2 fold increase | 140 | 79 | 288 |
| >2 fold decrease | 150 | 98 | 610 |
| >3 fold increase | 26 | 6 | 94 |
| >3 fold decrease | 27 | 22 | 203 |

Table 25 summarizes the results of common and unique transcripts differentially expressed in the resistance and progression models.

TABLE 25
Common and Unique transcripts differentially expressed
for each model

| | | Common | |
|---|---|---|---|
| | | transcripts | Unique transcripts |
| Comparisons | Type of model | for both models | for each model |
| CDr-HSD vs. | Resistance | 48 | 242 |
| CDs-HSD | | | |
| CDs-HSD vs. | Progression | | 128 |
| CDs-RD | | | |

The 48 common transcripts for these two models are listed in Table 26.

TABLE 26
Common Transcripts Differentially Expressed in Progression and Resistance Models

| | UniGene | UniGene | |
|---|---|---|---|
| Name | (rat) | (human) | Description and Gene Ontology |
| SERPINE2 | Rn.2271 | Hs.38449 | Serine proteinase inhibitor clade E member 2 |
| C20orf160 | Rn.6807 | Hs.382157 | C20orf160 predicted |
| | | | Cystein type endopeptidase |
| Unknown | Rn.33396 | n/a | unknown |
| LOC338328 | Rn.7294 | Hs.426410 | High density lipoprotein binding protein |
| PTPRR | Rn.6277 | Hs.506076 | Protein tyrosine phosphatase receptor type R, |
| LYPLA3 | Rn.93631 | Hs.632199 | Lysophosphilipase 3 |
| CYYR1 | Rn.1528 | Hs.37445 | Cysteine/tyrosine-rich 1 |
| | | | Membrane-associated protein |
| SOX17 | Rn.7884 | Hs.98367 | SRY-box gene 17 |
| LY6H | Rn.40119 | Hs.159590 | Lymphocyte antigen 6 complex, locus H |
| SEMA3G | Rn.32183 | Hs.59729 | Semaphorin 3G |
| C1QTNF1 | Rn.53880 | Hs.201398 | C1q and tumor necrosis factor related protein 1 |
| ADCY4 | Rn.1904 | Hs.443428 | Adenylate cyclase 4 |
| RBP7 | Rn.13092 | Hs.422688 | Retinol binding protein 7, |
| ADRB3 | Rn.10100 | Hs.2549 | Adrenergic, beta-3-, receptor |
| NR1H3 | Rn.11209 | Hs.438863 | Nuclear receptor subfamily, group H, member 3 |
| TMEFF1 | Rn.162809 | Hs.657066 | Transmembrane protein with EGF-like and two |
| | | | follistatin-like domains 1 |
| TIMP-4 | Rn.155651 | Hs.591665 | Tissue inhibitor of metalloproteinase 4 |
| CYP4F8 | Rn.10170 | Hs.268554 | Cytochrome P450, family 4, subfamily F, |
| | | | polypeptide 8 |
| FOLR1 | Rn.6912 | Hs.73769 | Folate receptor 1 |
| SCD | Rn.83595 | Hs.558396 | Stearoyl-CoA desaturase |
| KIAA2022 | Rn.62924 | Hs.124128 | DNA polymerase activity |
| GK | Rn.44654 | Hs.1466 | Glycerol kinase |
| OCLN | Rn.31429 | Hs.592605 | Occludin |
| SPINT2 | Rn.3857 | Hs.31439 | Serine peptidase inhibitor, Kunitz type, 2 |
| RBM24 | Rn.164640 | Hs.519904 | RNA binding motif protein 24 |
| SLC25A13 | Rn.14686 | Hs.489190 | Solute carrier family 25, member 13 (citrin) |
| TPMT | Rn.112598 | Hs.444319 | Thiopurine S-methyltransferase |
| KRT18 | Rn.103924 | Hs.406013 | Keratin 18 |
| unknown | Rn.153497 | n/a | unknown |
| C2orf40 | Rn.16593 | Hs.43125 | Chromosome 2 open reading frame 40 |
| LOC440335 | Rn.137175 | Hs.390599 | Hypothetical gene supported by BC022385 |
| BEXL1 | Rn.9287 | Hs.184736 | Brain expressed X-linked-like 1 |
| CYB561 | Rn.14673 | Hs.355264 | Cytochrome b-561 |
| AMOT | Rn.149241 | Hs.528051 | Angiomotin |
| SQLE | Rn.33239 | Hs.71465 | Squalene epoxidase |
| ANKRD6 | Rn.45844 | Hs.656539 | Ankyrin repeat domain 6 |
| CCDC8 | Rn.171055 | Hs.97876 | Coiled-coil domain containing 8 |
| KRT8 | Rn.11083 | Hs.533782 | Keratin 8 |
| WWC1 | Rn. 101912 | Hs.484047 | WW and C2 domain containing 1 |
| PFKP | Rn.2278 | Hs.26010 | Phosphofructokinase |
| PEBP1 | Rn.29745 | Hs.433863 | Phosphatidylethanolamine binding protein 1 |
| SLC7A1 | Rn.9439 | Hs.14846 | Solute carrier family 7 (cationic amino acid |
| | | | transporter, y+ system), member 1 |
| GSTM1 | Rn.625 | Hs.301961 | Glutathione S-transferase M1 |
| | | | Glutathione metabolism |
| CCL5 | Rn.8019 | Hs.514821 | Chemokine (C-C motif) ligand 5 |
| STEAP1 | Rn.51773 | Hs.61635 | Six transmembrane epithelial antigen of the |
| | | | prostate 1 |
| IAH1 | Rn.8209 | Hs.656852 | Isoamyl acetate-hydrolyzing esterase 1 homolog |
| | | | (S. cerevisiae) |
| GNA14 | Rn.35127 | Hs.657795 | Guanine nucleotide binding protein (G protein), |
| | | | alpha 14 |
| TMEM64 | Rn.164935 | Hs.567759 | transmembrane protein 64 |

Unique transcripts that show a change in expression of 3 fold or higher are listed in Table 27. These transcripts are unique in the sense that the changes of the expression level are observed only within one of the models described and as such, they may serve as markers to further study resistance against Type 2 Diabetes or progression and predisposition for the disease.

TABLE 27
Unique Transcripts Found in Epididymal Fat Tissue with Changes Greater than
3-Fold. (Appendix IV)

| | UniGene | UniGene | |
|---|---|---|---|
| Name | (rat) | (human) | Description and Gene Ontology |
| SDF2L1 | Rn.1414 | Hs.303116 | Stromal cell-derived factor 2-like 1 |
| CCL11 | Rn.10632 | Hs.54460 | Chemokine (C-C motif) ligand 11 |
| CNN1 | Rn.31788 | Hs.465929 | Calponin 1 |
| ZCD2 | Rn.24858 | Hs.556638 | Zinc finger, CDGSH-type domain 2 |
| CYR61 | Rn.22129 | Hs.8867 | Cysteine-rich, angiogenic inducer, 61 |
| GGH | Rn.10260 | Hs.78619 | Gamma-glutamyl hydrolase |
| TPM3 | Rn.17580 | Hs.645521 | Tropomyosin 3 |
| CSNK1A1 | Rn.23810 | Hs.654547 | Casein kinase 1, alpha 1 |
| PCDH7 | Rn.25383 | Hs.570785 | Protocadherin 7 |
| FHL2 | Rn.3849 | Hs.443687 | Four and a half LIM domains 2 |
| COL11A1 | Rn.260 | Hs.523446 | Collagen, type XI, alpha 1 |
| EMB | Rn.16221 | Hs.645309 | Embigin homolog (mouse) |
| ISG15 | Rn.198318 | Hs.458485 | ISG15 ubiquitin-like modifier |
| CRYAB | Rn.98208 | Hs.408767 | Crystallin, alpha B |
| ACADSB | Rn.44423 | Hs.81934 | Acyl-Coenzyme A dehydrogenase, |
| Unknown | Rn.164743 | n/a | Unknown |
| ABCA1 | Rn.3724 | Hs.429294 | ATP-binding cassette, sub-family A (ABC1), |
| | | | member 1 |
| Unknown | Rn.7699 | n/a | IMAGE clone: BC086433 |
| ACSM3 | Rn.88644 | Hs.653192 | Acyl-CoA synthetase medium-chain family |
| | | | member 3 |
| CHD2 | Rn.162437 | Hs.220864 | Chromodomain helicase DNA binding protein 2 |
| ACTA2 | Rn.195319 | Hs.500483 | Actin, alpha 2, smooth muscle, aorta |
| RAMP3 | Rn.48672 | Hs.25691 | Receptor (G protein-coupled) activity modifying |
| | | | protein 3 |
| DDEF1 | Rn.63466 | Hs.655552 | Development and differentiation enhancing factor 1 |
| NIPSNAP3A | Rn.8287 | Hs.591897 | Nipsnap homolog 3A (C. elegans) |
| Unknown | Rn.9546 | n/a | Unknown |
| GPR64 | Rn.57243 | Hs.146978 | G protein-coupled receptor 64 |
| SGCB | Rn.98258 | Hs.438953 | Sarcoglycan, beta |
| Unknown | Rn.146540 | n/a | Unknown |
| Unknown | Rn.199679 | n/a | Unknown |
| CALML3 | Rn.105124 | Hs.239600 | Calmodulin-like 3 |
| LOC645638 | Rn.41321 | Hs.463652 | Similar to WDNM1-like protein |
| RAB8B | Rn.10995 | Hs.389733 | RAB8B, a member RAS oncogene family |
| Unknown | Rn.6638 | n/a | Unknown |
| YTHDF2 | Rn.21737 | Hs.532286 | YTH domain family, member 2 |
| SCEL | Rn.34468 | Hs.534699 | Sciellin |
| BNC1 | Rn.26595 | Hs.459153 | Basonuclin 1 |
| FGL2 | Rn.64635 | Hs.520989 | Fibrinogen-like 2 |
| UPK1B | Rn.9134 | Hs.271580 | Uroplakin 1B |
| CTDSPL | Rn.37030 | Hs.475963 | CTD (carboxy-terminal domain, RNA |
| | | | polymerase II, polypeptide A) small phosphatase- |
| | | | like |
| PIK3R1 | Rn.163585 | Hs.132225 | Phosphoinositide-3-kinase, regulatory subunit 1 |
| | | | (p85 alpha) |
| POLA2 | Rn.153998 | Hs.201897 | Polymerase (DNA directed), alpha 2 (70 kD |
| | | | subunit) |
| SPTBN1 | Rn.93208 | Hs.659362 | Spectrin, beta, non-erythrocytic 1 |
| RTEL1 | Rn.98315 | Hs.434878 | Regulator of telomere elongation helicase 1 |
| MSLN | Rn.18607 | Hs.408488 | Mesothelin |
| ARVCF | Rn.220 | Hs.655877 | Armadillo repeat gene deletes in velocardiofacial |
| | | | syndrome |
| ALB | Rn.9174 | Hs.418167 | Albumin |
| SLC6A4 | Rn.1663 | Hs.591192 | Solute carrier family 6 (neurotransmitter |
| | | | transporter, serotonin), member 4 |
| SLC2A4 | Rn.1314 | Hs.380691 | Solute carrier family 2 (facilitated glucose |
| | | | transporter), member 4 |
| Unknown | Rn.26537 | n/a | Unknown |
| Unknown | Rn.44072 | n/a | Unknown |
| Unknown | Rn.199355 | n/a | Unknown |
| MRPL4 | Rn.13113 | Hs.279652 | Mitochondrial ribosomal protein L4 |
| GPR109A | Rn.79620 | Hs.524812 | G protein-coupled receptor 109A |

In summary, transcriptome/gene expression analyses were conducted on pancreatic and epididymal fat tissue for the Cohen rat models. Transcripts differentially expressed for both tissues have been characterized as described above. For selected transcripts (14 transcripts for pancreatic tissue and 48 transcripts for epididymal fat tissue), the microarray results have been confirmed by quantitative PCR.

It is to be understood that while the invention has been described in conjunction with the detailed description thereof, the foregoing description is intended to illustrate and not limit the scope of the invention, which is defined by the scope of the appended claims. Other aspects, advantages, and modifications are within the ambit of the following claims.

What is claimed is:

1. A method of diagnosing or identifying type 2 Diabetes, one or more complications related to type 2 Diabetes, or a pre-diabetic condition in a subject, comprising:
a. measuring an effective amount of one or more T2DBMARKERS or a metabolite thereof in a sample from the subject; and
b. comparing the amount to a reference value, wherein an increase or decrease in the amount of the one or more T2DBMARKERS relative to the reference value indicates that the subject suffers from the type 2 Diabetes, one or more complications related to type 2 Diabetes, or the pre-diabetic condition.

2. The method of claim 1, wherein the reference value comprises an index value, a value derived from one or more Diabetes risk prediction algorithms or computed indices, a value derived from a subject not suffering from type 2 Diabetes or the pre-diabetic condition, a value derived from a subject diagnosed with or identified as suffering from type 2 Diabetes or the pre-diabetic condition, or a value derived from a subject previously diagnosed with or identified as suffering from one or more complications related to type 2 Diabetes.

3. The method according to claim 1, wherein the subject comprises one who has been previously diagnosed as having type 2 Diabetes, one or more complications related to type 2 Diabetes, or a pre-diabetic condition, one who has not been previously diagnosed as having type 2 Diabetes, one or more complications related to type 2 Diabetes, or a pre-diabetic condition, or one who is asymptomatic for the type 2 Diabetes, one or more complications related to type 2 Diabetes or a pre-diabetic condition.

4. A method for monitoring the progression of type 2 Diabetes, one or more complications relating to type 2 Diabetes, or a pre-diabetic condition in a subject, comprising
a. detecting an effective amount of one or more T2DBMARKERS in a first sample from the subject at a first period of time;
b. detecting an effective amount of one or more T2DBMARKERS in a second sample from the subject at a second period of time; and
c. comparing the amounts of the one or more T2DBMARKERS detected in step (a) to the amount detected in step (b), or to a reference value.

5. The method of claim 4, wherein the monitoring comprises evaluating changes in the risk of developing type 2 Diabetes, one or more complications relating to type 2 Diabetes, or the pre-diabetic condition.

6. The method of claim 4, wherein the subject comprises one who has previously been treated for the type 2 Diabetes, one or more complications relating to type 2 Diabetes, or the pre-diabetic condition, one who has not been previously treated for the type 2 Diabetes, one or more complications relating to type 2 Diabetes, or the pre-diabetic condition, or one who has not been previously diagnosed with or identified as suffering from type 2 Diabetes, one or more complications relating to type 2 Diabetes, or the pre-diabetic condition.

7. The method of claim 4, wherein the first sample is taken from the subject prior to being treated for the type 2 Diabetes, one or more complications relating to type 2 Diabetes, or the pre-diabetic condition.

8. The method of claim 4, wherein the second sample is taken from the subject after being treated for the type 2 Diabetes, one or more complications relating to type 2 Diabetes, or the pre-diabetic condition.

9. The method of claim 4, wherein the monitoring further comprises selecting a treatment regimen for the subject and/or monitoring the effectiveness of a treatment regimen for type 2 Diabetes, one or more complications relating to type 2 Diabetes, or the pre-diabetic condition.

10. The method of claim 9, wherein the treatment for the type 2 Diabetes, one or more complications relating to type 2 Diabetes, or the pre-diabetic condition comprises exercise regimens, dietary supplements, surgical intervention, diabetes-modulating agents, or combinations thereof.

11. The method of claim 4, wherein the reference value comprises an index value, a value derived from one or more Diabetes risk prediction algorithms or computed indices, a value derived from a subject not suffering from type 2 Diabetes, one or more complications relating to type 2 Diabetes, or a pre-diabetic condition, or a value derived from a subject diagnosed with or identified as suffering from type 2 Diabetes, one or more complications relating to type 2 Diabetes, or a pre-diabetic condition.

12. A method of treating a subject diagnosed with or identified as suffering from type 2 Diabetes, one or more complications relating to type 2 Diabetes, or a pre-diabetic condition comprising:
a. detecting an effective amount of one or more T2DBMARKERS or metabolites thereof present in a first sample from the subject at a first period of time; and
b. treating the subject with one or more diabetes-modulating agents until the amounts of the one or more T2DBMARKERS or metabolites thereof return to a reference value measured in one or more subjects at low risk for developing type 2 Diabetes, one or more complications relating to type 2 Diabetes, or a pre-diabetic condition, or a reference value measured in one or more subjects who show improvements in Diabetes risk factors as a result of treatment with the one or more diabetes-modulating agents.

13. The method of claim 12, wherein the one or more diabetes-modulating agents comprise sulfonylureas, biguanides, insulin, insulin analogs, peroxisome proliferator-activated receptor-γ (PPAR-γ) agonists, dual-acting PPAR agonists, insulin secretagogues, analogs of glucagon-like peptide-1 (GLP-1), inhibitors of dipeptidyl peptidase IV, pancreatic lipase inhibitors, α-glucosidase inhibitors, or combinations thereof.

14. The method of claim 12, wherein the improvements in Diabetes risk factors as a result of treatment with one or more diabetes-modulating agents comprise a reduction in body mass index (BMI), a reduction in blood glucose levels, an increase in insulin levels, an increase in HDL levels, a reduction in systolic and/or diastolic blood pressure, or combinations thereof.

15. A kit comprising T2DBMARKER detection reagents that detect one or more T2DBMARKERS, a sample derived from a subject having normal glucose levels, and optionally instructions for using the reagents in the method of any one of claims 1, 4, and 12, wherein the T2DBMARKER detection reagents comprise the isolated antibody of claim 17.

16. The kit of claim 15, wherein the detection reagents further comprise one or more antibodies or fragments thereof, one or more aptamers, one or more oligonucleotides, or combinations thereof.

17. An isolated antibody or antigen-binding fragment thereof, comprising a human constant region and an antigen-binding region, wherein the antigen-binding region binds one or more T2DBMARKERS or a metabolite thereof.

18. The isolated antibody of claim 17, wherein the antigen-binding region binds one or more amino acid residues of SEQ ID NO: 1.

19. The isolated antibody of claim 17, which is recombinant.
